(19) United States
(12) Patent Application Publication
(10) Pub. No.: US 2024/0010697 A1
(43) Pub. Date: Jan. 11, 2024

(54) MODIFIED INTERLEUKIN 2 (IL-2) POLYPEPTIDES, AND METHODS OF MAKING AND USING THE SAME

(71) Applicant: CYTIMM THERAPEUTICS, INC., San Diego, CA (US)

(72) Inventors: HAINING HUANG, San Diego, CA (US); XIAO XU, San Diego, CA (US); YU FENG, San Diego, CA (US); GUILIANA MOGNOL, San Diego, CA (US); CAN JIN, San Diego, CA (US); DIANA GUIMET, San Diego, CA (US)

(73) Assignee: CYTIMM THERAPEUTICS, INC., San Diego, CA (US)

(21) Appl. No.: 18/248,729

(22) PCT Filed: Oct. 13, 2021
(86) PCT No.: PCT/US2021/054809

§ 371 Date: Apr. 12, 2023

Related U.S. Application Data
(60) Provisional application No. 63/091,679, filed on Oct. 14, 2020.

(51) Int. Cl. C07K 14/55; A61K 47/10; C12N 15/63; A61K 45/06; A61P 37/02

(52) U.S. Cl. CPC C07K 14/55; A61K 47/10; C12N 15/63; A61K 45/06; A61P 37/02

(57) ABSTRACT

This disclosure relates, inter alia to: modified interleukin 2 (IL-2) polypeptides; RNA polynucleotides, DNA polynucleotides, non-viral vectors, and viral vectors encoding such modified IL-2 polypeptides; methods of making such modified IL-2 polypeptides and RNA polynucleotides, DNA polynucleotides, non-viral vectors, and viral vectors encoding same; and methods of using such modified IL-2 polypeptides and RNA polynucleotides, DNA polynucleotides, non-viral vectors, and viral vectors encoding same.

### CROSS REFERENCE TO RELATED APPLICATIONS

This application claims priority to U.S. provisional patent application No. 63/091,679, filed on Oct. 14, 2020, the content and disclosure of which is incorporated by reference in its entirety for all purposes.

### TECHNICAL FIELD

This disclosure relates to, inter alia, modified interleukin 2 (IL-2) polypeptides and/or conjugates comprising the modified IL-2 polypeptides, polynucleotides encoding such modified IL-2 polypeptides, such as DNA polynucleotides and/or RNA polynucleotides, vectors harboring such polynucleotides, such as viral or non-viral vectors that facilitate expression of said modified IL-2 polypeptides in vitro and/or in vivo from host cells harboring such vectors, and uses thereof, such as methods for treating certain diseases, conditions, or disorders.

### BACKGROUND

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins which modulate the development and differentiation of T and B lymphocytes, cell of the monocytic lineage, neutrophils, basophils, eosinophils, megakaryocytes, and hematopoietic cells. Interleukins are produced by helper CD4 T and B lymphocytes, monocytes, macrophages, endothelial cells, and other tissue residents. Interleukin 2 (IL-2) is a pleiotropic type-1 cytokine whose structure comprises a 15.5 kDa four a-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and, to a lesser extent, by CD8+ cells, Natural Killer (NK) cells, and NK T (NKT) cells, activated dendritic cells (DCs), and mast cells. IL-2 signaling occurs through interaction with specific combinations of IL-2 receptor (IL-2R) subunits, IL-2Ra (also known as CD25), IL-2RP (also known as CD122), and IL-2Rγ (also known as CD 132). Interaction of IL-2 with the IL-2Rα forms the “low-affinity” IL-2 receptor complex with a K_{d} of about 10⁻⁸ M. Interaction of IL-2 with IL-2Rβ and IL-2Rγ forms the “intermediate-affinity” IL-2 receptor complex with a K_{d} of about 10⁻⁹ M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, such as CD4+CD25+Foxp3+ regulatory T (Treg) cells, mediate maintenance of immune homeostasis by suppression of effector cells such as CD8+ T cells, helper cells such as CD4+ 0, Th2, and Th1 7 cells, B cells, NK cells, and NK T cells. In some instances, Treg cells are generated from the thymus (tTreg cells) or are induced from naive T cells in the periphery (pTreg cells). In some cases, Treg cells are considered as a predominant mediator of peripheral tolerance. Indeed, in one study, transfer of CD25-depleted peripheral CD4+ T cells produced a variety of autoimmune diseases in nude mice, whereas cotransfer of CD4+CD25+ T cells suppressed the development of autoimmunity (Sakaguchi et al., J. Immunol. (1995)). Augmentation of the Treg cell population down-regulates effector T cell proliferation and suppresses autoimmunity and T cell anti-tumor responses.

Clinical use of interleukin-2 (IL-2) for treatment of many disease and disorders, such as cancers and autoimmune and inflammatory diseases has been mainly limited by toxicity and short half-life in vivo (Pachella et al., J Adv Pract Oncol (2015); Lotze et al. (1985) J. Immunol (1985)). It has been observed that toxicity was markedly reduced in animals deficient in CD25 (IL-2 receptor α unit, IL-2Rα) Boyman, et al., J Immunol (2009)) PEGylation, the covalent attachment of Polyethylene glycol (PEG) to therapeutics, has also been shown to overcome certain obstacles such as rapid body clearance, aggregation and enzymatic degradation, in certain instances Maiser et al., Biotechnol Bioeng (2014)).

WO 2019/028419 A1 and WO 2019/028425 A1 disclose certain interleukin (IL) conjugates (e.g., IL-2 conjugates) and use in the treatment of certain indications. Also described in WO 2019/028419 A1 and WO 2019/028425 A1 are pharmaceutical compositions and kits comprising one or more of the interleukin conjugates (e.g., IL-2 conjugates).

There exists a need for improved modified interleukin 2 (IL-2) polypeptides including such modified interleukin 2 (IL-2) polypeptides that comprise conjugates. The disclosure provided herein and throughout addresses this and other related needs.

### SUMMARY

The disclosure provided herein and throughout is directed, inter alia, to: modified interleukin 2 (IL-2) polypeptides; RNA polynucleotides, DNA polynucleotides, non-viral vectors, and viral vectors encoding such modified IL-2 polypeptides; methods of making such modified IL-2 polypeptides and RNA polynucleotides, DNA polynucleotides, non-viral vectors, and viral vectors encoding such IL-2 polypeptides; and methods of using such modified IL-2 polypeptides and RNA polynucleotides, DNA polynucleotides, non-viral vectors, and viral vectors encoding such IL-2 polypeptides.

In certain embodiments, which may be combined with other embodiments disclosed herein and throughout, a modified interleukin 2 (IL-2) polypeptide is provided wherein the modified interleukin 2 (IL-2) polypeptide comprises an amino acid sequence having at least 80% identity to SEQ ID NO:1 or SEQ ID NO:2, wherein the modified IL-2 polypeptide comprises a substitution with a natural or unnatural amino acid at a position selected from the group consisting of L18, L19, N29, Y31, V69, N71, Q74, N88, V91, I128 and a combination thereof, wherein said modified IL-2 polypeptide: a) has enhanced binding to an interleukin 2 receptor α (IL-2Rα) compared to an IL-2 polypeptide without the substitution; and/or b) has enhanced binding to an interleukin 2 receptor αβγ (IL-2Rαβγ) compared an IL-2 polypeptide without the substitution; and/or b) has enhanced binding to cells expressing an interleukin 2 receptor αβγ (IL-2Rαβγ) compared to an IL-2 polypeptide without the substitution; and/or c) has enhanced receptor signaling potency via IL-2Rαβγ compared to an IL-2 polypeptide without the substitution; and/or e) has an enhanced ratio of IL-2Rαβγ receptor signaling potency to IL-2Rβγ receptor signaling potency compared to the ratio of IL-2Rαβγ receptor signaling potency to IL-2Rβγ receptor signaling potency of an IL-2 polypeptide without the substitution; and/or f) is configured to be conjugated to a conjugating moiety; and/or g) is conjugated to a conjugating moiety; and/or h) combinations of a) through g).

In certain embodiments, which may be combined with other embodiments disclosed herein and throughout, a modified interleukin 2 (IL-2) polypeptide is provided, wherein the modified interleukin 2 (IL-2) polypeptide comprises: a) a substitution with cysteine, lysine, histidine, arginine, aspartic acid, glutamic acid, serine, threonine, alanine, tryptophan, isoleucine, phenylalanine, or tyrosine at position N29; and/or b) a substitution with cysteine, lysine, histidine, arginine, aspartic acid, glutamic acid, serine, threonine, alanine, tryptophan, isoleucine, or phenylalanine at position Y31.

In certain embodiments, which may be combined with other embodiments disclosed herein and throughout, a modified interleukin 2 (IL-2) polypeptide is provided wherein the modified interleukin 2 (IL-2) polypeptide comprises the substitution N29C.

In certain embodiments, which may be combined with other embodiments disclosed herein and throughout, a modified interleukin 2 (IL-2) polypeptide is provided wherein the modified interleukin 2 (IL-2) polypeptide comprises the substitution Y31C.

In certain embodiments, which may be combined with other embodiments disclosed herein and throughout, a modified interleukin 2 (IL-2) polypeptide is provided wherein the modified interleukin 2 (IL-2) polypeptide comprises a substitution with lysine, cysteine, histidine, arginine, aspartic acid, glutamic acid, serine, threonine, alanine, methionine, tryptophan, isoleucine, phenylalanine, proline, or tyrosine at one or more positions selected from the group consisting of L18, L19, V69, Q74, N88, V91, and I128.

In certain embodiments, which may be combined with other embodiments disclosed herein and throughout, a modified interleukin 2 (IL-2) polypeptide is provided wherein the modified interleukin 2 (IL-2) polypeptide comprises a substitution selected from the group consisting of Y31C.

In certain embodiments, which may be combined with other embodiments disclosed herein and throughout, a modified interleukin 2 (IL-2) polypeptide is provided wherein the modified interleukin 2 (IL-2) polypeptide is conjugated to a conjugating moiety selected from the group consisting of a water-soluble polymer, a lipid, a peptide, a protein, a polypeptide, and combinations thereof.

In certain embodiments, which may be combined with other embodiments disclosed herein and throughout, a modified interleukin 2 (IL-2) polypeptide is provided wherein the modified interleukin 2 (IL-2) polypeptide comprises is conjugated to a polyethylene glycol.

In certain embodiments, which may be combined with other embodiments disclosed herein and throughout, a modified interleukin 2 (IL-2) polypeptide is provided wherein the modified interleukin 2 (IL-2) polypeptide comprises a mutation selected from the group consisting of N29C, N30C, Y31C, E100C, N119C, T123C, S127C, or T131C, wherein the polypeptide is pegylated at the N29C, N30C, Y31C, E100C, N119C, T123C, S127C, or T131C site.

In certain embodiments, which may be combined with other embodiments disclosed herein and throughout, a modified interleukin 2 (IL-2) polypeptide is provided wherein the modified interleukin 2 (IL-2) polypeptide comprises a N29C or Y31C mutation.

In certain embodiments, which may be combined with other embodiments disclosed herein and throughout, a modified interleukin 2 (IL-2) polypeptide is provided wherein the modified interleukin 2 (IL-2) polypeptide comprises: a) a substitution with lysine, cysteine, histidine, arginine, aspartic acid, glutamic acid, serine, threonine, alanine, tryptophan, isoleucine, phenylalanine, or tyrosine at a position selected from the group consisting of N29, N30, Y31 and combinations thereof; or b) a substitution with lysine, cysteine, histidine, arginine, aspartic acid, glutamic acid, serine, threonine, alanine, tryptophan, isoleucine, phenylalanine, or tyrosine at a position selected from the group consisting of N30, Y31, and combinations thereof.

In certain embodiments, which may be combined with other embodiments disclosed herein and throughout, a modified interleukin 2 (IL-2) polypeptide is provided wherein the modified interleukin 2 (IL-2) polypeptide comprises: a) a substitution with a natural amino acid or an unnatural amino acid at one or more positions selected from the group consisting of N29, N30, Y31, and is: (i) unconjugated; (ii) conjugated to; or (iii) configured to be conjugated to; one or more water-soluble polymers, lipids, proteins, or peptides at one or more positions selected from the group consisting of N29, N30, Y31, E100, N119, T123, S127, T131; and/or b) a substitution with a natural amino acid or an unnatural amino acid at a position selected from the group consisting of N29, N30, Y31, and is: (i) unconjugated; (ii) conjugated to; or (iii) configured to be conjugated to; one or more water-soluble polymers, lipids, proteins, or peptides at one or more positions selected from the group consisting of N29, N30, Y31; and/or c) a substitution with a natural amino acid or an unnatural amino acid at a position selected from the group consisting of N29, N30, Y31 and a combination thereof, and is: (i) unconjugated; (ii) conjugated to; or (iii) configured to be conjugated to; one or more water-soluble polymers, lipids, proteins, or peptides at the N terminal and/or C terminal of the modified IL-2 polypeptide.

In certain embodiments, which may be combined with other embodiments disclosed herein and throughout, a modified interleukin 2 (IL-2) polypeptide is provided wherein the modified interleukin 2 (IL-2) polypeptide comprises: a) a substitution with cysteine at one or more positions selected from the group consisting of N29, N30, Y31; and/or b) a substitution with cysteine at one or more positions selected from the group consisting of N30, Y31; and/or c) comprises a substitution with cysteine at a position of Y31; and/or f) comprises a substitution with cysteine at a position of N30.

In certain embodiments, which may be combined with other embodiments disclosed herein and throughout, a modified interleukin 2 (IL-2) polypeptide is provided wherein the modified interleukin 2 (IL-2) polypeptide comprises one or more substitutions with a natural amino acid or an unnatural amino acid at a position within IL-2Rα interaction region, and/or IL-2Rβ interaction region and/or IL-2Rγ interaction region.

In certain embodiments, which may be combined with other embodiments disclosed herein and throughout, a modified interleukin 2 (IL-2) polypeptide is provided wherein the modified interleukin 2 (IL-2) polypeptide comprises one or more substitutions with a natural amino acid or an unnatural amino acid at a position within IL-2Rβ interaction region and/or IL-2Rγ interaction region.

In certain embodiments, which may be combined with other embodiments disclosed herein and throughout, a modified interleukin 2 (IL-2) polypeptide is provided wherein the modified interleukin 2 (IL-2) polypeptide comprises one or more substitutions with a natural amino acid or an unnatural amino acid at a position selected from the group consisting of L18, L19, V69, Q74, N88, V91, I128, and a combination thereof.

In certain embodiments, which may be combined with other embodiments disclosed herein and throughout, a modified interleukin 2 (IL-2) polypeptide is provided wherein the modified interleukin 2 (IL-2) polypeptide comprises one or more substitutions with lysine, cysteine, histidine, arginine, aspartic acid, glutamic acid, serine, threonine, alanine, methionine, tryptophan, isoleucine, phenylalanine, proline, or tyrosine at a position selected from the group consisting of L18, L19, V69, Q74, N88, V91, I128, and a combination thereof.

In certain embodiments, which may be combined with other embodiments disclosed herein and throughout, a modified interleukin 2 (IL-2) polypeptide is provided wherein the modified interleukin 2 (IL-2) polypeptide comprises: a) a substitution with methionine at a position L18; and/or b) a substitution with serine at a position of L19; and/or c) a substitution with cysteine at position of Y31, and/or d) comprises a substitution with alanine at a position of V69; and/or e) comprises a substitution with proline at a position of Q74; and/or f) comprises a substitution with arginine, aspartic acid, glutamic acid, lysine at a position of N88; and/or g) comprises a substitution with arginine at a position of N88; and/or h) comprises a substitution with aspartic acid at a position of N88; i) comprises a substitution with glutamic acid at a position of N88; j) comprises a substitution with lysine at a position of N88; k) comprises a substitution with lysine at a position of V91; 1) comprises a substitution with threonine at a position of I128; and/or m) combinations of a) through 1).

In certain embodiments, which may be combined with other embodiments disclosed herein and throughout, a modified interleukin 2 (IL-2) polypeptide is provided wherein the modified interleukin 2 (IL-2) polypeptide comprises: a) a substitution with a natural amino acid at a position within IL-2Rα interaction region and a substitution with a natural amino acid at a position within IL-2Rβ interaction region; and/or b) a substitution with a natural amino acid at a position within IL-2Rα interaction region and a substitution with a natural amino acid at a position within IL-2Rγ interaction region; and/or c) a substitution with a natural amino acid at a position within IL-2Rα interaction region, a substitution with a natural amino acid at a position within IL-2Rβ interaction region and a substitution with a natural amino acid at a position within IL-2Rγ interaction region.

In certain embodiments, which may be combined with other embodiments disclosed herein and throughout, a modified interleukin 2 (IL-2) polypeptide is provided wherein the modified interleukin 2 (IL-2) polypeptide has increased binding to an IL-2Rα and/or IL-2Rαβγ compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the binding affinity of the modified IL-2 polypeptide to an IL-2Rα and/or IL-2Rαβγ is increased from about 10% to about 100%, or is increased from about 1-fold to about 100,000-fold or more.

In certain embodiments, which may be combined with other embodiments disclosed herein and throughout, a modified interleukin 2 (IL-2) polypeptide is provided wherein the modified IL-2 polypeptide has increased binding to IL-2Rα expressing cells and/or IL-2Rαβγ expressing cells compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has increased binding to IL-2Rα expressing cells and/or IL-2Rαβγ expressing cells that is increased from about 10% to about 100%, or is increased from about 1-fold to about 100,000-fold or more.

In certain embodiments, which may be combined with other embodiments disclosed herein and throughout, a modified interleukin 2 (IL-2) polypeptide is provided wherein the modified IL-2 polypeptide has reduced internalization by IL-2Rα expressing cells and/or IL-2Rαβγ expressing cells compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has internalization by IL-2Rα expressing cells and/or IL-2Rαβγ expressing cells that is from about 10% to about 100%, or is increased from about 1-fold to about 100,000-fold or more.

In certain embodiments, which may be combined with other embodiments disclosed herein and throughout, a modified interleukin 2 (IL-2) polypeptide is provided wherein the modified IL-2 polypeptide has no detectable internalization by IL-2Rα expressing cells and/or IL-2Rαβγ expressing cells.

In certain embodiments, which may be combined with other embodiments disclosed herein and throughout, a modified interleukin 2 (IL-2) polypeptide is provided wherein the modified IL-2 polypeptide has increased receptor signaling potency to IL-2Rαβγ compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has increased binding to an IL-2Rα compared to an IL-2 polypeptide comprising an amino acid sequence set forth in SEQ ID NO:1 or SEQ ID NO:2 without the substitution and has increased receptor signaling potency to IL-2Rαβγ compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has increased binding to an IL-2Rα and/or IL-2Rαβγ, and increased binding on IL-2Rα expressing cells and/or IL-2Rαβγ expressing cells compared an IL-2 polypeptide comprising an amino acid sequence set forth in SEQ ID NO:1 or SEQ ID NO:2 without the substitution, and has reduced internalization by IL-2Rα expressing cells and/or IL-2Rαβγ expressing cells compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has: (i) increased binding to an IL-2Rα and/or IL-2Rαβγ; (ii) increased binding on IL-2Rα expressing cells and/or IL-2Rαβγ expressing cells compared to an IL-2 polypeptide comprising an amino acid sequence set forth in SEQ ID NO:1 or SEQ ID NO:2 without the substitution; (iii) no detectable internalization by IL-2Rα expressing cells and/or IL-2Rαβγ expressing cells compared to an IL-2 polypeptide comprising an amino acid sequence set forth in SEQ ID NO:1 or SEQ ID NO:2 without the substitution; (iv) and increased receptor signaling potency to IL-2Rαβγ compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has: reduced binding level to an interleukin 2 receptor β (IL-2Rβ) or an interleukin 2 receptor γ (IL-2Rγ) compared to an IL-2 polypeptide comprising an amino acid sequence set forth in SEQ ID NO:1 or SEQ ID NO:2 without the substitution; and/or reduced receptor signaling potency to IL-2Rβγ compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has lower receptor signaling potency to IL-2Rβγ compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has: (i) lower binding level to an IL-2Rβ or an IL-2Rγ compared to an IL-2 polypeptide comprising an amino acid sequence set forth in SEQ ID NO:1 or SEQ ID NO:2 without the substitution; and (ii) lower receptor signaling potency to IL-2Rβγ compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has increased ratio between its signaling potency to IL-2Rαβγ and the signaling potency to IL-2Rβγ compared to an IL-2 polypeptide comprising an amino acid sequence set forth in SEQ ID NO:1 or SEQ ID NO:2 without the substitution.

In certain embodiments, which may be combined with other embodiments disclosed herein and throughout, a modified interleukin 2 (IL-2) polypeptide is provided wherein the modified IL-2 polypeptide has increased ratio between its signaling potency to IL-2Rαβγ and the signaling potency to IL-2Rβγ is more than 1-fold, more than 10-fold, more than 100-fold, more than 1,000-fold, more than 10,000-fold, more than 100,000-fold.

In certain embodiments, which may be combined with other embodiments disclosed herein and throughout, a modified interleukin 2 (IL-2) polypeptide is provided wherein the modified IL-2 polypeptide comprises an N terminal deletion, wherein said deletion comprises a deletion of one or more of amino acid residues 1 through 30, inclusive, that are present in the corresponding IL-2 modified polypeptide that does not comprise said N-terminal deletion.

In certain embodiments, which may be combined with other embodiments disclosed herein and throughout, a modified interleukin 2 (IL-2) polypeptide is provided wherein the modified IL-2 polypeptide comprises a C terminal deletion, wherein said deletion comprises a deletion of one or more of amino acid residues 114 through 134, inclusive, that are present in the corresponding IL-2 modified polypeptide that does not comprise said C-terminal deletion.

In certain embodiments, which may be combined with other embodiments disclosed herein and throughout, a modified interleukin 2 (IL-2) polypeptide is provided wherein the modified IL-2 polypeptide comprises a N terminal deletion and a C terminal deletion.

In certain embodiments, which may be combined with other embodiments disclosed herein and throughout, a modified interleukin 2 (IL-2) polypeptide is provided wherein the modified IL-2 polypeptide is a part of a fusion polypeptide comprising an additional amino acid sequence.

In certain embodiments, which may be combined with other embodiments disclosed herein and throughout, a modified interleukin 2 (IL-2) polypeptide is provided wherein the modified IL-2 polypeptide comprises a recombinant fusion protein comprising the modified IL-2 polypeptide and an additional amino acid sequence.

In certain embodiments, which may be combined with other embodiments disclosed herein and throughout, a modified interleukin 2 (IL-2) polypeptide is provided wherein the N terminus or the C terminus of the modified IL-2 polypeptide is fused to an additional amino acid sequence.

In certain embodiments, which may be combined with other embodiments disclosed herein and throughout, a modified interleukin 2 (IL-2) polypeptide is provided wherein the N terminus or the C terminus of the modified IL-2 polypeptide is fused to an additional amino acid sequence, wherein said additional amino acid sequence comprises an antibody sequence or a portion or a fragment thereof.

In certain embodiments, which may be combined with other embodiments disclosed herein and throughout, a modified interleukin 2 (IL-2) polypeptide is provided wherein the N terminus or the C terminus of the modified IL-2 polypeptide is fused to an additional amino acid sequence, wherein said additional amino acid sequence comprises an Fc portion of an antibody or a portion or a fragment thereof.

In certain embodiments, which may be combined with other embodiments disclosed herein and throughout, a modified interleukin 2 (IL-2) polypeptide is provided wherein the modified IL-2 polypeptide is isolated.

In certain embodiments, which may be combined with other embodiments disclosed herein and throughout, a modified interleukin 2 (IL-2) polypeptide is provided wherein the modified IL-2 polypeptide is expressed from a vector comprising a polynucleotide sequence that encodes the modified IL-2 polypeptide.

In certain embodiments, which may be combined with other embodiments disclosed herein and throughout, a modified interleukin 2 (IL-2) polypeptide is provided wherein the modified IL-2 polypeptide is expressed from a vector comprising a polynucleotide sequence that encodes the modified IL-2 polypeptide, wherein said vector is an RNA vector, a DNA, a viral vector, or a non-viral vector.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a polypeptide, a protein, or a peptide.

In certain embodiments, which may be combined with other embodiments disclosed herein and throughout, a modified interleukin 2 (IL-2) polypeptide is provided wherein the modified IL-2 polypeptide is conjugated to one or more water-soluble polymers, lipids, proteins, or peptides through one or more covalent bonds.

In certain embodiments, which may be combined with other embodiments disclosed herein and throughout, a modified interleukin 2 (IL-2) polypeptide is provided wherein the modified IL-2 polypeptide is conjugated to one or more water-soluble polymers, lipids, proteins, or peptides through one or more non-covalent bonds.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a substituted natural amino acid or unnatural amino acid at a position selected from the group consisting of L18, L19, N30, Y31, V69, Q74, N88, V91, I128, E100, N119, T123, S127, T131, and combinations thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a substituted natural amino acid at a position selected from the group consisting of L18, L19, N30, Y31, V69, Q74, N88, V91, I128, E100, N119, T123, S127, T131, and combinations thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a substituted lysine, cysteine, histidine, arginine, aspartic acid, glutamic acid, serine, threonine, alanine, tryptophan, isoleucine, phenylalanine, or tyrosine at a position selected from the group consisting of L18, L19, N30, Y31, V69, Q74, N88, V91, I128, E100, N119, T123, S127, T131, and combinations thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a substituted cysteine at a position selected from the group consisting of L18, L19, N30, Y31, V69, Q74, N88, V91, I128, E100, N119, T123, S127, T131, and combinations thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a substituted natural amino acid or unnatural amino acid at a position selected from the group consisting of L18, L19, N30, Y31, V69, Q74, N88, V91, I128, E100, N119, T123, S127, T131, and combinations thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a substituted natural amino acid at a position selected from the group consisting of L18, L19, N30, Y31, V69, Q74, N88, V91, I128, E100, N119, T123, S127, T131, and combinations thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a substituted lysine, cysteine, histidine, arginine, aspartic acid, glutamic acid, serine, threonine, alanine, tryptophan, isoleucine, phenylalanine, or tyrosine at a position selected from the group consisting of L18, L19, N30, Y31, V69, Q74, N88, V91, I128, E100, N119, T123, S127, T131, and combinations thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a substituted cysteine at a position selected from the group consisting of L18, L19, N30, Y31, V69, Q74, N88, V91, I128, E100, N119, T123, S127, T131, and combinations thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a single amino acid residue of the modified IL-2 polypeptide.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via: i) the alpha amino group of the N-terminal amino acid residue of the modified IL-2 polypeptide; ii) the epsilon amino group of a lysine amino acid residue of the modified IL-2 polypeptide; or iii) an N-glycosylation site or O-glycosylation site of the modified IL-2 polypeptide.

In certain embodiments, which may be combined with other embodiments disclosed herein and throughout, a modified interleukin 2 (IL-2) polypeptide is provided wherein the modified IL-2 polypeptide is covalently conjugated to a water-soluble polymer, a lipid, a protein, or a peptide through a linker.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a single amino acid residue located within the modified IL-2 polypeptide.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the single amino acid residue is located within the additional amino acid sequence.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the additional amino acid sequence comprises an antibody sequence or a portion or a fragment thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the additional amino acid sequence comprises a Fc portion of an antibody.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the single amino acid residue is: i) the alpha amino group of the N-terminal amino acid residue of the fusion polypeptide; ii) the epsilon amino group of a lysine amino acid residue of the fusion polypeptide; or iii) an N-glycosylation site or O-glycosylation site of the fusion polypeptide.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the fusion polypeptide is covalently conjugated to the water-soluble polymer, a lipid, a protein, or a peptide through a linker.

In certain embodiments, which may be combined with other embodiments disclosed herein and throughout, a modified interleukin 2 (IL-2) polypeptide is provided wherein the modified IL-2 polypeptide is conjugated to a water-soluble polymer.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or combinations thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a PEG molecule.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a linear PEG molecule.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a branched PEG molecule.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a branched PEG molecule comprising about three to about ten PEG chains emanating from a central core group.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a branched PEG molecule, wherein the branched PEG molecule is a star PEG comprising from about 10 to about 100 PEG chains emanating from a central core group.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a branched PEG molecule, wherein the branched PEG molecule is a comb PEG comprising multiple PEG chains grafted onto a polymer backbone.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a PEG molecule, wherein the PEG molecule has a range of molecular weight from about 300 g/mol to about 10,000,000 g/mol.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a PEG molecule, wherein the PEG molecule has an average molecular weight from about 5,000 Daltons to about 1,000,000 Daltons.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a PEG molecule, wherein the PEG molecule has an average molecular weight of from about 20,000 Daltons to about 30,000 Daltons.

In certain embodiments, which may be combined with other embodiments disclosed herein and throughout, a modified interleukin 2 (IL-2) polypeptide is provided wherein the modified IL-2 polypeptide is conjugated to a water-soluble polymer comprising a PEG molecule, wherein the PEG molecule is a monodisperse, uniform, or discrete PEG molecule.

In certain embodiments, which may be combined with other embodiments disclosed herein and throughout, a modified interleukin 2 (IL-2) polypeptide is provided wherein the modified IL-2 polypeptide is conjugated to a water-soluble polymer, wherein the water-soluble polymer comprises a polysaccharide.

In certain embodiments, which may be combined with other embodiments disclosed herein and throughout, a modified interleukin 2 (IL-2) polypeptide is provided wherein the modified IL-2 polypeptide is conjugated to a lipid.

In certain embodiments, which may be combined with other embodiments disclosed herein and throughout, a modified interleukin 2 (IL-2) polypeptide is provided wherein the modified IL-2 polypeptide is conjugated to a lipid, wherein the lipid comprises a fatty acid.

In certain embodiments, which may be combined with other embodiments disclosed herein and throughout, a modified interleukin 2 (IL-2) polypeptide is provided wherein the modified IL-2 polypeptide is conjugated to a protein.

In certain embodiments, which may be combined with other embodiments disclosed herein and throughout, a modified interleukin 2 (IL-2) polypeptide is provided wherein the modified IL-2 polypeptide is conjugated to a protein, wherein the protein comprises an antibody or a binding fragment thereof.

In certain embodiments, which may be combined with other embodiments disclosed herein and throughout, a modified interleukin 2 (IL-2) polypeptide is provided wherein the modified IL-2 polypeptide is conjugated to an Fc portion of an antibody or a fragment thereof.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide that is indirectly bound to the substituted natural amino acid or unnatural amino acid of the modified IL-2 polypeptide through a linker.

In certain embodiments, which may be combined with other embodiments disclosed herein and throughout, a modified interleukin 2 (IL-2) polypeptide is provided wherein the modified IL-2 polypeptide is conjugated to a water-soluble polymer, a lipid, a protein, or a peptide that is directly bound to the substituted natural amino acid or unnatural amino acid of the modified IL-2 polypeptide.

In certain embodiments, which may be combined with other embodiments disclosed herein and throughout, a modified interleukin 2 (IL-2) polypeptide is provided wherein the modified IL-2 polypeptide, wherein the modified IL-2 polypeptide has a half-life in vivo from about 5 minutes to about 10 days.

In certain embodiments, which may be combined with other embodiments disclosed herein and throughout, a modified interleukin 2 (IL-2) polypeptide is provided wherein the modified IL-2 polypeptide is selected from the group consisting of ACT5200, ACT5201, ACT5210, ACT5211, ACT5212, ACT522S0, ACT522S1, ACT5230, ACT5231, ACT5260, ACT5261, ACT5270, ACT5271, ACT5280, ACT5281, ACT5290, and ACT5291.

In certain embodiments, which may be combined with other embodiments disclosed herein and throughout, a pharmaceutical composition is provided comprising an effective amount of a modified IL-2 polypeptide as disclosed herein and throughout and a pharmaceutically acceptable carrier or excipient.

In certain embodiments, which may be combined with other embodiments disclosed herein and throughout, a pharmaceutical composition is provided comprising an effective amount of a modified IL-2 polypeptide as disclosed herein and throughout, wherein the pharmaceutical composition further comprises another active ingredient.

In certain embodiments, which may be combined with other embodiments disclosed herein and throughout, a pharmaceutical composition is provided comprising an effective amount of a modified IL-2 polypeptide as disclosed herein and throughout, wherein the pharmaceutical composition further comprises one or more additional ingredients, wherein the one or more active ingredients comprises: (i) an anti-inflammatory substance or an anti-autoimmune substance; (ii) an anti-neoplasm substance; (iii) an anti-infectious disease substance; and/or (iv) an immune deficiency disorder.

In certain embodiments, which may be combined with other embodiments disclosed herein and throughout, a pharmaceutical composition is provided comprising an effective amount of a modified IL-2 polypeptide as disclosed herein and throughout, wherein the pharmaceutical composition is for use in treating or preventing a disease or disorder in a subject having, or suspected of having, the disease or disorder.

In certain embodiments, which may be combined with other embodiments disclosed herein and throughout, a pharmaceutical composition is provided comprising an effective amount of a modified IL-2 polypeptide as disclosed herein and throughout, wherein the pharmaceutical composition is for use in treating or preventing a disease or disorder in a subject having, or suspected of having, the disease or disorder, wherein the disease or disorder comprises an inflammatory disease or disorder or an autoimmune disease or disorder.

In certain embodiments, which may be combined with other embodiments disclosed herein and throughout, a pharmaceutical composition is provided comprising an effective amount of a modified IL-2 polypeptide as disclosed herein and throughout, wherein the pharmaceutical composition is for use in treating or preventing a disease or disorder in a subject having, or suspected of having, the disease or disorder, wherein the disease or disorder comprises a proliferation disease or disorder.

In certain embodiments, which may be combined with other embodiments disclosed herein and throughout, a pharmaceutical composition is provided comprising an effective amount of a modified IL-2 polypeptide as disclosed herein and throughout, wherein the pharmaceutical composition is for use in treating or preventing a disease or disorder in a subject having, or suspected of having, the disease or disorder, wherein the disease or disorder comprises an infectious disease or disorder.

In certain embodiments, which may be combined with other embodiments disclosed herein and throughout, a pharmaceutical composition is provided comprising an effective amount of a modified IL-2 polypeptide as disclosed herein and throughout, wherein the pharmaceutical composition is for use in treating or preventing a disease or disorder in a subject having, or suspected of having, the disease or disorder, wherein the disease or disorder comprises an immune deficiency disorder.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises an inflammatory disease or disorder or an autoimmune disease or disorder.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises a proliferation disease or disorder.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises an infectious disease or disorder.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises an immune deficiency disease or disorder.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the subject is a human.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the subject is a non-human mammal.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises a proliferation disease or disorder, wherein the proliferation disorder comprises a tumor.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises a proliferation disease or disorder wherein the proliferation disorder comprises a cancer.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises a proliferation disease or disorder, wherein the proliferation disorder comprises a solid tumor or a cancer.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises a proliferation disease or disorder, wherein the proliferation disorder comprises a solid tumor or a cancer, wherein the solid tumor or the cancer is selected from the group consisting of: Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), and Desmoplastic small round cell tumor and Liposarcoma.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises a proliferation disease or disorder, wherein the proliferation disorder comprises a tumor or a cancer, wherein the tumor or cancer is a hematological malignancy.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises a proliferation disease or disorder, wherein the proliferation disorder comprises a tumor or a cancer, wherein the tumor or cancer is a hematological malignancy selected from the group consisting of: myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenström's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schöner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenström's macroglobulinaemia, Alpha heavy chain disease, Gamma heavy chain disease, Franklin's disease, Immunoproliferative small intestinal disease, Mediterranean disease, Malignant immunoproliferative disease, unspecified, and Immunoproliferative disease NOS.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises an inflammatory disease or disorder or an autoimmune disease or disorder, wherein the inflammatory disease or disorder or the autoimmune disease or disorder is selected from the group consisting of: inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, and transplantation rejection.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises an infectious disease or disorder, wherein the infectious disease is selected from the group consisting of: Acinetobacter infections, Actinomycosis, African sleeping sickness (African trypanosomiasis), AIDS (acquired immunodeficiency syndrome), Amoebiasis, Anaplasmosis, Angiostrongyliasis Anisakiasis, Anthrax, Arcanobacterium haemolyticum infection, Argentine hemorrhagic fever, Ascariasis, Aspergillosis Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus (children and babies), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, SARS (severe acute respiratory syndrome), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, and Zygomycosis.

In certain embodiments, which may be combined with other embodiments disclosed herein and throughout, a method is provided for treating or preventing a disease or a disorder in a subject having, or suspected of having, the disease or disorder, comprising administering to said subject an effective amount of a modified IL-2 polypeptide as disclosed herein and throughout, or a pharmaceutical composition as disclosed herein and throughout, wherein the disease or disorder comprises an immune deficiency disease or disorder, wherein the immune deficiency disease or disorder is selected from the group consisting of: Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs), and Lymphopenia.

In certain embodiments, which may be combined with other embodiments disclosed herein and throughout, is provided an effective amount of a modified IL-2 polypeptide, RNA polynucleotide, a DNA polynucleotide, a non-viral vector, or a viral vector encoding a modified IL-2 polypeptide, each independently as disclosed herein and throughout, for the manufacture of a medicament for treating or preventing a disease or a disorder in a subject.

In certain embodiments, which may be combined with other embodiments disclosed herein and throughout, is provided an effective amount of a modified IL-2 polypeptide, RNA polynucleotide, a DNA polynucleotide, a non-viral vector, or a viral vector encoding a modified IL-2 polypeptide, each independently as disclosed herein and throughout, for the manufacture of a medicament for treating or preventing a disease or a disorder in a subject, wherein the disease or disorder is selected from the group consisting of: an inflammatory disease or disorder; an autoimmune disease or disorder; a proliferative disease or disorder; an infectious disease or disorder; and an immune deficiency disease or disorder.

In certain embodiments, which may be combined with other embodiments disclosed herein and throughout, is provided a method of expanding a Treg cell population, comprising contacting a cell population with an effective amount of a modified IL-2 polypeptide, RNA polynucleotide, a DNA polynucleotide, a non-viral vector, or a viral vector encoding a modified IL-2 polypeptide, each independently as disclosed herein and throughout, for a time sufficient to induce formation of a complex with an IL-2Rαβγ, thereby stimulating the expansion of the Treg cell population.

In certain embodiments, which may be combined with other embodiments disclosed herein and throughout, is provided a method of expanding a Treg cell population, comprising contacting a cell population with an effective amount of a modified IL-2 polypeptide, RNA polynucleotide, a DNA polynucleotide, a non-viral vector, or a viral vector encoding a modified IL-2 polypeptide, each independently as disclosed herein and throughout, for a time sufficient to induce formation of a complex with an IL-2Rαβγ, thereby stimulating the expansion of the Treg cell population with reduced cell death by 10% to 100%.

In certain embodiments, which may be combined with other embodiments disclosed herein and throughout, is provided a method of expanding a Treg cell population, comprising contacting a cell population with an effective amount of a modified IL-2 polypeptide, RNA polynucleotide, a DNA polynucleotide, a non-viral vector, or a viral vector encoding a modified IL-2 polypeptide, each independently as disclosed herein and throughout, wherein the method causes expansion of CD25+T regulatory (Treg) cells by at least 1-fold, 10-fold, 100-fold, 1,000-fold, 10⁴-fold, 10⁵ fold, 10⁶-fold, 10⁷-fold, 10⁸-fold, or 10⁹-fold greater that the expansion of CD25+ Treg cells caused with an IL-2 polypeptide comprising an amino acid sequence set forth in SEQ ID NO:1 or SEQ ID NO:2 without the substitution.

In certain embodiments, which may be combined with other embodiments disclosed herein and throughout, is provided a method of expanding a Treg cell population, comprising contacting a cell population with an effective amount of a modified IL-2 polypeptide, RNA polynucleotide, a DNA polynucleotide, a non-viral vector, or a viral vector encoding a modified IL-2 polypeptide, each independently as disclosed herein and throughout, wherein the effective amount causes an increased the percentage of Treg cells in the T cell population after incubation with the effective amount, compared with an IL-2 polypeptide comprising an amino acid sequence set forth in SEQ ID NO:1 or SEQ ID NO:2 without the substitution, and percentage of the Treg cells is about or at least 0.01%, 0.1%, 1%, 5%, 10%, 20%, 30%, 40%, 50%, 60%, 70%, 80%, 90% or more.

In certain embodiments, which may be combined with other embodiments disclosed herein and throughout, is provided a method of expanding a Treg cell population, comprising contacting a cell population with an effective amount of a modified IL-2 polypeptide, RNA polynucleotide, a DNA polynucleotide, a non-viral vector, or a viral vector encoding a modified IL-2 polypeptide, each independently as disclosed herein and throughout, wherein the method is conducted in vivo.

In certain embodiments, which may be combined with other embodiments disclosed herein and throughout, is provided a method of expanding a Treg cell population, comprising contacting a cell population with an effective amount of a modified IL-2 polypeptide, RNA polynucleotide, a DNA polynucleotide, a non-viral vector, or a viral vector encoding a modified IL-2 polypeptide, each independently as disclosed herein and throughout, wherein the method is conducted in vitro.

In certain embodiments, which may be combined with other embodiments disclosed herein and throughout, is provided a method of expanding a Treg cell population, comprising contacting a cell population with an effective amount of a modified IL-2 polypeptide, RNA polynucleotide, a DNA polynucleotide, a non-viral vector, or a viral vector encoding a modified IL-2 polypeptide, each independently as disclosed herein and throughout, wherein the method is conducted ex vivo.

In certain embodiments, which may be combined with other embodiments disclosed herein and throughout, is provided a use of an effective amount a modified IL-2 polypeptide, RNA polynucleotide, a DNA polynucleotide, a non-viral vector, or a viral vector encoding a modified IL-2 polypeptide, each independently as disclosed herein and throughout, for the manufacture of a medicament for expanding a Treg cell in a cell population.

In certain embodiments, which may be combined with other embodiments disclosed herein and throughout, is provided a use of an effective amount a modified IL-2 polypeptide, RNA polynucleotide, a DNA polynucleotide, a non-viral vector, or a viral vector encoding a modified IL-2 polypeptide, each independently as disclosed herein and throughout, for the manufacture of a medicament for expanding a Treg cell in a cell population, wherein the Treg cells are expanded in a subject.

In certain embodiments, which may be combined with other embodiments disclosed herein and throughout, is provided modified interleukin 2 (IL-2) polypeptides, in which the modified IL-2 polypeptides comprise an amino acid sequence having at least 80% identity to SEQ ID NO:1 or SEQ ID NO:2 and where the modified IL-2 polypeptides include one or more substitutions of a natural amino acid or an unnatural amino acid at a position selected from the group consisting of L18, L19, N29, N30, Y31, V69, N71, Q74, N88, V91, E100, N119, T123, S127, I128, and T131, in which the modified IL-2 polypeptide may be unconjugated or conjugated to a water-soluble polymer, lipid, or protein or peptide. A modified IL-2 polypeptide as provided herein and throughout can have one or more of the following properties with respect to a comparable IL-2 polypeptide identical in sequence to the modified IL-2 polypeptide except that it does not include the one or more amino acid substitutions: a) enhanced binding to an interleukin 2 receptor α (IL-2Rα) without the one or more substitutions; b) enhanced binding to an interleukin 2 receptor αβγ (IL-2Rαβγ); c) reduced internalization by cells expressing the interleukin 2 receptor α (IL-2Rα) or interleukin 2 receptor αβγ (IL-2Rαβγ) receptor; d) enhanced signaling potency via IL-2Rαβγ; and/or e) enhanced ratio of IL-2Rαβγ/IL-2Rβγ signaling potency. In certain embodiments, a modified IL-2 polypeptide as provided herein and throughout further includes an amino acid substitution to cysteine at one or more of N29, N30, Y31, Q74, K76, E100, N119, T123, S127, or T131. In certain embodiments, a modified IL-2 polypeptide as provided herein and throughout further includes a mutation with respect to SEQ ID NO:1 or SEQ ID NO:2 selected from the group consisting of N29C, N30C, Y31C, Q74C, K76C, E100C, N119C, T123C, S127C, or T131C, and the IL-2 is conjugated to a lipid, sugar, peptide, protein, or polymer via the cysteine at the mutated site. In certain embodiments, a modified IL-2 polypeptide can have a mutation with respect to SEQ ID NO:1 or SEQ ID NO:2 selected from the group consisting of N29C, N30C, Y31C, Q74C, K76C, E100C, N119C, T123C, S127C, or T131C, where the IL-2 is conjugated to polyethylene glycol via the cysteine at the mutated amino acid position. Modified IL-2 polypeptides as provided herein and throughout can have at least 80%, at least 85%, at least 90%, or at least 95% identity to any of SEQ ID NO:1 or SEQ ID NO:2. In certain embodiments, a modified IL-2 polypeptide as provided herein and throughout can have at least 95%, at least 96%, at least 97%, at least 98%, or at least 99% identity to SEQ ID NO:1 or SEQ ID NO:2.

In certain embodiments, which may be combined with other embodiments disclosed herein and throughout, is provided a modified IL-2 polypeptide as provided herein and throughout comprises the sequence of SEQ ID NO:1, SEQ ID NO:2, or SEQ ID NO:3 with the exception that the modified IL-2 polypeptide includes a substitution of a natural or unnatural amino acid at one or more positions selected from the group consisting of L18, L19, N29, Y31, V69, N71, N88, V91, I128, and T131, and optionally further includes a mutation selected from the group consisting of N30C, Q74C, K76C, E100C, N119C, T123C, S127C, and T131C. In some examples, a modified IL-2 polypeptide includes one or more of the mutations L18M, L19S, N29C, Y31C, V69A, N71C, Q74P, N88R, V91K, and I128T and further includes a mutation selected from the group consisting of N30C, Q74C, K76C, E100C, N119C, T123C, S127C, and T131C, where the N30C, Q74C, K76C, E100C, N119C, T123C, S127C, or T131C mutation is the site of conjugation to a water-soluble polymer, sugar, lipid, protein, or peptide. In certain embodiments, a modified IL-2 polypeptide as provided herein and throughout can include a mutation at one or more of the amino acid positions L18, L19, N29, Y31, V69, N71, N88, V91, I128, and T131 and can further include one or more of the mutations N30C, Q74C, K76C, E100C, N119C, T123C, S127C, and T131C, where one or more of the mutated residues N30C, Q74C, K76C, E100C, N119C, T123C, S127C, and T131C is the site of conjugation to a water-soluble polymer, sugar, lipid, protein or peptide. In various embodiments the modified IL-2 polypeptide is conjugated to polyethylene glycol (PEG).

In certain embodiments, which may be combined with other embodiments disclosed herein and throughout, is provided a modified IL-2 polypeptide having at least 80% identity to SEQ ID NO:1 or SEQ ID NO:2 that includes a mutation with respect to SEQ ID NO:1 selected from the group consisting of N29C, N30C, Y31C, N71C, Q74C, K76C, E100C, N119C, T123C, S127C, and T131C. The modified IL-2 polypeptide can be conjugated to a water-soluble polymer, sugar, lipid, protein, or peptide at the N29C, N30C, Y31C, N71C, Q74C, K76C, E100C, N119C, T123C, S127C, or T131C mutation site. In certain embodiments, the modified IL-2 polypeptide is conjugated to PEG at the cysteine at amino acid position 29, 30, 31, 74, 76, 100, 119, 123, 127, or 131. In various embodiments, the modified IL-2 polypeptide conjugated to PEG at the cysteine at amino acid position 29, 30, 31, 74, 76, 100, 119, 123, 127, or 131 has an increased half-life in human serum with respect to a comparable IL-2 polypeptide that is not conjugated to PEG at the cysteine at amino acid position 29, 30, 31, 74, 76, 100, 119, 123, 127, or 131. The modified IL-2 polypeptide conjugated to a water-soluble polymer, sugar, lipid, protein, or peptide at the N29C, N30C, Y31C, Q74C, K76C, E100C, N119C, T123C, S127C, or T131C can further include one or more mutations, such as any disclosed herein and throughout.

In certain embodiments, which may be combined with other embodiments disclosed herein and throughout, is provided a modified IL-2 polypeptide conjugate, which comprises a modified IL-2 polypeptide, as described above, that is conjugated to a water-soluble polymer, a lipid, a polypeptide, e.g., a protein, or a peptide. The modified IL-2 polypeptide can include a mutation to cysteine of any of the amino acids N29, N30, Y31, N71, Q74, K76, E100, N119, T123, S127, or T131, where the water-soluble polymer, sugar, lipid, protein, or peptide is conjugated to the modified IL-2 polypeptide. In certain embodiments, the conjugate comprises a modified polypeptide as described herein and throughout conjugated via a cysteine residue to PEG. The modified IL-2 polypeptide can have at least 80%, at least 85%, at least 90%, at least 95%, at least 96%, at least 97%, at least 98%, or at least 99% to SEQ ID NO:1 or SEQ ID NO:2, and can further include a substitution, with respect to SEQ ID NO:1 or SEQ ID NO:2 at one or more or amino acids L18, L19, N29, Y31, V69, N71, N88, V91, and I128. In certain embodiments, the modified IL-2 conjugate can include an IL-2 polypeptide having any of the mutations L18M, L19S, N29C, Y31C, V69A, N71C, Q74P, N88R, V91K, and I128T.

In certain embodiments, which may be combined with other embodiments disclosed herein and throughout, is provided a nucleic acid molecule encoding a modified IL-2 polypeptide. In certain embodiments, the nucleic acid molecule may comprise RNA polynucleotide sequence, or DNA polynucleotide sequence. In certain embodiments, the nucleic acid molecule may be a viral vector or a non-viral vector. In certain embodiments, such a vector comprises one or more of an expression control sequence and a selectable or detectable marker. Further provided is a modified IL-2 polypeptide as disclosed herein and throughout, or a nucleic acid molecule encoding a modified IL-2 polypeptide as disclosed herein and throughout, complexed with, bound to, or enclosed in a delivery compound or structure such as a liposome or nanoparticle.

In certain embodiments, which may be combined with other embodiments disclosed herein and throughout, is provided a pharmaceutical composition comprising a modified IL-2 polypeptide, a nucleic acid molecule encoding a modified IL-2 polypeptide, or a modified IL-2 polypeptide conjugate, and a pharmaceutically acceptable carrier or excipient.

In certain embodiments, which may be combined with other embodiments disclosed herein and throughout, is provided a method for treating or preventing a disease or a disorder, e.g., an infectious or autoimmune disease or disorder, in a subject in need, comprising administering to said subject an effective amount of a modified IL-2 polypeptide, a nucleic acid molecule encoding a modified IL-2 polypeptide, a modified IL-2 polypeptide conjugate, or a pharmaceutical composition including any of these.

In certain embodiments, which may be combined with other embodiments disclosed herein and throughout, is provided a use of an effective amount of a modified IL-2 polypeptide, a nucleic acid molecule encoding a modified IL-2 polypeptide, or a modified IL-2 polypeptide conjugate, as described herein and throughout, for the manufacture of a medicament for treating or preventing a disease or a disorder, e.g., an infectious or autoimmune disease or disorder, in a subject.

In certain embodiments, which may be combined with other embodiments disclosed herein and throughout, is provided a method of expanding a Treg cell, CD4⁺ helper cell, CD8⁺ effector naive and memory cell, Natural Killer (NK) cell, Natural killer T (NKT) cell, or other IL-2Rαβγ expressing cell population, which comprises contacting a cell population with an effective amount of a modified IL-2 polypeptide or a modified IL-2 polypeptide conjugate as described herein and throughout, or a pharmaceutical composition comprising the modified IL-2 polypeptide or modified IL-2 polypeptide conjugate, for a time sufficient to induce formation of a complex with an IL-2Rαβγ, thereby stimulating the expansion of the Treg cell, CD4⁺ helper cell, CD8⁺ effector naive and memory cell, NK cell, and/or NKT cell population.

In certain embodiments, which may be combined with other embodiments disclosed herein and throughout, is provided a method of expanding a Treg cell, CD4⁺ helper cell, CD8⁺ effector naive and memory cell, Natural Killer (NK) cell, Natural killer T (NKT) cell, or other IL-2Rαβγ expressing cell population, which comprises contacting a cell population with an effective amount of a modified IL-2 polypeptide or a modified IL-2 polypeptide conjugate thereof, a modified IL-2 polypeptide-encoding RNA polynucleotide, a modified IL-2 polypeptide-encoding DNA polynucleotide, a modified IL-2 polypeptide-encoding viral vector, or a modified IL-2 polypeptide-encoding non-viral vector, as described above, or a pharmaceutical composition comprising the modified IL-2 polypeptide or modified IL-2 polypeptide conjugate for a time sufficient to induce formation of a complex with an IL-2Rαβγ, thereby stimulating the expansion of the Treg cell, CD4⁺ helper cell, CD8⁺ effector naive and memory cell, NK cell, and/or NKT cell population with reduced cell death by 10% to 100%.

In certain embodiments, which may be combined with other embodiments disclosed herein and throughout, is provided a use of an effective amount of a modified IL-2 polypeptide or a modified IL-2 polypeptide conjugate, as described above, for the manufacture of a medicament for expanding a Treg cell, CD4⁺ helper cell, CD8⁺ effector naive and memory cell, Natural Killer (NK) cell, Natural killer T (NKT) cell, or other IL-2Rαβγ expressing cell in a cell population.

Other embodiments, aspects, and advantages of the disclosure will be apparent from the embodiments and examples provided herein and throughout.

For the sake of brevity, the disclosures of the publications cited in this specification, including patents and patent applications, are hereby incorporated by reference in their entireties.

### BRIEF DESCRIPTION OF THE DRAWINGS

FIG. 1A illustrates the sequence of an exemplary recombinant human IL-2 (rhIL-2) polypeptide comprising a mutation from cysteine to serine at position 125 (SEQ ID NO:1). The amino acid positions selected to be pegylated through individual cysteine substitution and/or the positions selected to modulate (e.g., enhance) IL-2Rα interaction and/or modulate (e.g., diminish) IL-2Rβγ interaction by mutation are labeled by superscripted numbers. FIG. 1B illustrates the 3-D structure of IL-2 and receptor IL-2Rαβγ complex derived from PDB structure 2b5i. See e.g., The Protein Data Bank H. M. Berman, J. Westbrook, Z. Feng, G. Gilliland, T. N. Bhat, H. Weissig, I. N. Shindyalov, P. E. Bourne (2000) Nucleic Acids Research, 28: 235-242. doi:10.1093/nar/28.1.235. The positions indicated in FIG. 1A are shown as red spheres.

FIG. 2 illustrates exemplary or typical profile of chromatography and SDS-PAGE analysis for exemplary IL-2 muteins and PEG-conjugates. FIG. 2A shows chromatography of two exemplary modified IL-2 polypeptides, ACT5210 (Y31C+L18ML19S) and ACT5211 (Y31C-PEG20+L18ML19S), by Superdex 75 Increase column. FIG. 2B shows SDS-PAGE analysis of exemplary modified IL-2 polypeptides ACT5210, ACT5211, ACT5230, ACT5231 fractions contained in eluates obtained by applying the polypeptides to SP Sepharose FF columns followed by Superdex 75 Increase columns (mutein names correspond to those provided in Table 2).

FIGS. 3A-3C provide exemplary sensorgrams of the indicated exemplary IL-2 muteins and corresponding PEG-conjugates binding with IL-2 receptors obtained by Octet Qke (ForteBio, San Jose, CA). FIG. 3A depicts binding to IL-2Rα. FIG. 3B depicts binding to the indicated IL-2 receptor complexes. FIG. 3C provides a table containing various measured binding and/or kinetic parameters (e.g., ka, kd, kD) for IL-2, ACT5210, and ACT5211.

FIGS. 4A-4C provide binding data of the indicated exemplary IL-2 muteins on IL-2Rαβγ-expressing CTL-2 cells. FIGS. 4A and 4B illustrate that the numerous exemplary muteins, with or without PEGylation display enhanced binding to IL-2Rαβγ-expressing CTLL2 and CD25+ T cells, as indicated. The enhanced binding is also seen in the PEGylated Y31C-containing muteins ACT5211 and ACT5261 (FIG. 4C).

FIG. 5 illustrates in vitro half-life of the indicated exemplary muteins, as well as IL-R2, when co-cultured with IL-2Rα-expressing T cells. At least ACT5201, ACT5210, ACT5211, and ACT5231 displayed an extended half-life relative to that observed for rhIL-2.

FIGS. 6A and 6B provide stimulation and ex vivo expansion of Treg cells observed upon treatment with the indicated muteins, as well as rhIL-2. FIG. 6A provides results observed with rhIL2, ACT5211, and ACT5231. FIG. 6B provides results observed with rhIL2, ACT5211, and ACT5261.

FIGS. 7A and 7B each illustrate T cell activation as measured by pSTAT assay for the indicated IL-2 muteins.

FIG. 8 provides binding data and EC50 measurements obtained by performing ELISA assays using the indicated IL-2 muteins.

FIG. 9 provides mouse pharmacokinetic (PK) data obtained upon administration of ACT2511 (mpk=milligrams per kilogram (mg/kg)).

FIGS. 10A and 10B provide pharmacodynamic (PD) data obtained upon administration of ACT2511 to mice.

### DETAILED DESCRIPTION

### Exemplary Techniques

The practice of the disclosure provided herein and throughout may employ, unless otherwise indicated, conventional techniques of molecular biology (including recombinant techniques), microbiology, cell biology, biochemistry, immunology, and pharmacology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); and Remington, The Science and Practice of Pharmacy, 20¹ ed., (Lippincott, Williams & Wilkins 2003).

### Definitions

Unless defined otherwise, all technical and scientific terms used herein and throughout have the same meaning as is commonly understood by one of ordinary skill in the art to which this invention belongs. All patents, patent applications (published or unpublished), and other publications referred to herein and throughout are incorporated by reference in their entireties. If a definition set forth in this section is contrary to or otherwise inconsistent with a definition set forth in the patents, applications, published applications and other publications that are incorporated by reference, the definition set forth in this section prevails over the definition that is incorporated herein by reference.

As used herein and throughout, “a” or “an” means “at least one” or “one or more.” Similarly, any singular use of any word, include plural referents unless expressly and unequivocally limited to one referent. The term “or” is used in an inclusive sense, i.e., equivalent to “and/or,” unless the context dictates otherwise. As used herein and throughout, the terms “comprise,” “include,” and grammatical variants thereof are intended to be non-limiting, such that recitation of items in a list is not to the exclusion of other like items that can be added to the listed items. Section divisions in the specification are provided for the convenience of the reader only and do not limit any combination of elements discussed. In case of any contradiction or conflict between material incorporated by reference and the expressly described content provided herein, the expressly described content controls.

The terms “polypeptide,” “oligopeptide,” “peptide,” and “protein” are used interchangeably herein and throughout to refer to polymers of amino acids of any length, e.g., at least 5, 6, 7, 8, 9, 10, 20, 30, 40, 50, 100, 200, 300, 400, 500, 1,000 or more amino acids. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein and throughout, the terms “variant” is used in reference to polypeptides that have some degree of amino acid sequence identity to a parent polypeptide sequence. A variant is similar to a parent sequence, but has at least one substitution, deletion or insertion in their amino acid sequence that makes them different in sequence from a parent polypeptide. Additionally, a variant may retain the functional characteristics of the parent polypeptide, e.g., maintaining a biological activity that is at least 50%, 60%, 70%, 80%, 90%, 95%, 98%, or 99% of that of the parent polypeptide.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule, and can be an immunoglobulin of any class, e.g., IgG, IgM, IgA, IgD and IgE. IgY, which is the major antibody type in avian species such as chicken, is also included within the definition. As used herein and throughout, the term encompasses not only intact polyclonal or monoclonal antibodies, but also fragments thereof (such as Fab, Fab′, F(ab′)2, Fv), single chain (ScFv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

As used herein and throughout, the term “antigen” refers to a target molecule that is specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

As used herein and throughout, the term “epitope” refers to a portion of an antigen, e.g., a peptide sequence of at least about 3 to 5, preferably about 5 to 10 or 15, and not more than about 1,000 amino acids (or any integer there between), which defines a sequence that by itself or as part of a larger sequence, binds to an antibody generated in response to such a sequence. There is no critical upper limit to the length of the fragment, which may, for example, comprise nearly the full-length of the antigen sequence, or even a fusion protein comprising two or more epitopes from the target antigen. An epitope for use in the subject invention is not limited to a peptide having the exact sequence of the portion of the parent protein from which it is derived, but also encompasses sequences identical to the native sequence, as well as modifications to the native sequence, such as deletions, additions and substitutions (conservative in nature).

As used herein and throughout, the terms “modified IL-2 polypeptide”, “mutein”, and “IL-2 mutein”, each in singular or plural form, and the like, be used interchangeably herein and throughout, refer to modified versions, mutants, and/or variants of one or more reference IL-2 polypeptides. In certain embodiments, such modified IL-2 polypeptides, muteins, IL-2 muteins, or variants comprise one or more substitutions, deletions, additions, and or fusions with one or more additional polypeptides, as disclosed herein and throughout.

As used herein and throughout, the terms “IL-2 polypeptide conjugate”, “IL-2 conjugate”, “modified IL-2 polypeptide conjugate”, “modified IL-2 polypeptide conjugate”, and “IL-2 mutein”, each in singular or plural form, and the like, used interchangeable herein and throughout, refer to IL-2 polypeptides, modified IL-2 polypeptides, IL-2 muteins, or variants which are conjugated to, or are configured for conjugation to, one or more polymers or other chemical moieties that modulate the binding, binding affinity, activations, and/or other biological, biochemical, or physiological consequence of an interaction of a corresponding IL-2 polypeptide or modified IL-2 polypeptide that is not so-conjugated. Such IL-2 polypeptide conjugates, IL-2 conjugates, modified IL-2 polypeptide conjugates, modified IL-2 polypeptide conjugates, IL-2 variants, and IL-2 muteins, each in singular or plural form, and the like, are understood to, themselves, be and/or comprise modified IL-2 polypeptides, IL-2 muteins, IL-2 variants, and the like, as disclosed herein and throughout.

In certain embodiments, “conjugate”, “conjugation moiety”, “conjugating moiety”, each in singular or plural form, used interchangeably herein and throughout refer to chemical entities that may be associated with and/or bonded to, such as through covalent bonds and/or non-covalent bonds, that may be employed to generate IL-2 polypeptide conjugates, IL-2 conjugates, modified IL-2 polypeptide conjugates, modified IL-2 polypeptide conjugates, IL-2 variants, and/or IL-2 muteins. In certain embodiments, such conjugates, conjugation moieties, conjugating moieties, may comprise polymers or other chemical moieties, such as water-soluble polymers, lipids, peptides, and/or polypeptides. In certain embodiments, such conjugates, conjugation moieties, conjugating moieties, may comprise polymers or other chemical moieties, such as water-soluble polymers, lipids, peptides, and/or polypeptides. In certain embodiments, such conjugates, conjugation moieties, conjugating moieties comprise one or more polyethylene glycol (PEG) moieties or polymeric units.

In certain embodiments, IL-2 polypeptide conjugates, IL-2 conjugates, modified IL-2 polypeptide conjugates, modified IL-2 polypeptide conjugates, IL-2 variants, and/or IL-2 muteins comprise one or more conjugates, conjugation moieties, and/or conjugating moieties. In certain embodiments, IL-2 polypeptide conjugates, IL-2 conjugates, modified IL-2 polypeptide conjugates, modified IL-2 polypeptide conjugates, IL-2 variants, and/or IL-2 muteins comprise one or more such as water-soluble polymers, lipids, peptides, and/or polypeptides. In certain embodiments, IL-2 polypeptide conjugates, IL-2 conjugates, modified IL-2 polypeptide conjugates, modified IL-2 polypeptide conjugates, IL-2 variants, and/or IL-2 muteins comprise one or more polyethylene glycol (PEG) moieties or polymeric units.

As used herein and throughout, the term “specifically binds” refers to the binding specificity of a specific binding pair. Recognition by an antibody of a particular target in the presence of other potential targets is one characteristic of such binding. Specific binding involves two different molecules wherein one of the molecules specifically binds with the second molecule through chemical or physical means. The two molecules are related in the sense that their binding with each other is such that they are capable of distinguishing their binding partner from other assay constituents having similar characteristics. The members of the binding component pair are referred to as ligand and receptor (anti-ligand), specific binding pair (SBP) member and SBP partner, and the like. A molecule may also be an SBP member for an aggregation of molecules; for example an antibody raised against an immune complex of a second antibody and its corresponding antigen may be considered to be an SBP member for the immune complex.

“Polynucleotide,” or “nucleic acid,” used interchangeably herein and throughout, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein and throughout, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

As used herein, the term “homologue” is used to refer to a nucleic acid which differs from a naturally occurring nucleic acid (e.g., the “prototype” or “wild-type” nucleic acid) by minor modifications to the naturally occurring nucleic acid, but which maintains the basic nucleotide structure of the naturally occurring form. Such changes include, but are not limited to: changes in one or a few nucleotides, including deletions (e.g., a truncated version of the nucleic acid) insertions and/or substitutions. A homologue can have enhanced, decreased, or substantially similar properties as compared to the naturally occurring nucleic acid. A homologue can be complementary or matched to the naturally occurring nucleic acid. Homologues can be produced using techniques known in the art for the production of nucleic acids including, but not limited to, recombinant DNA techniques, chemical synthesis, etc.

As used herein and throughout, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

With reference to a sequence-modified polypeptide, the term “comparable polypeptide” refers to a polypeptide that is identical in sequence to the subject sequence-modified polypeptide except for the sequence modification(s) or mutation(s) (substitution, deletion, or insertion of one or more amino acids) of the sequence-modified polypeptide. A “comparable polypeptide” with respect to a modified polypeptide that includes a conjugated moiety, such as a conjugated sugar, lipid, protein, peptide, or polymer (e.g., polyethylene glycol), is a polypeptide that has identical sequence to the subject modified polypeptide but is not conjugated to the sugar, lipid, protein, peptide, or polymer.

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5×Denhardt's solution, 5× SSPE, 0.2% SDS at 42° C., followed by washing in 0.2× SSPE, 0.2% SDS, at 42° C. High stringency conditions can be provided, for example, by hybridization in 50% formamide, 5×Denhardt's solution, 5× SSPE, 0.2% SDS at 42° C., followed by washing in 0.1× SSPE, and 0.1% SDS at 65° C. Low stringency hybridization refers to conditions equivalent to hybridization in 10% formamide, 5×Denhardt's solution, 6× SSPE, 0.2% SDS at 22° C., followed by washing in 1× SSPE, 0.2% SDS, at 37° C. Denhardt's solution contains 1% Ficoll, 1% polyvinylpyrolidone, and 1% bovine serum albumin (BSA). 20× SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art.

As used herein and throughout, a “vector” refers to discrete elements comprising polynucleotide sequences encoding a polypeptide of interest, such as a polypeptide comprising a modified IL-2 polypeptide disclosed herein. In certain embodiments, such vectors may be employed to introduce heterologous DNA or RNA polynucleotides encoding such modified IL-2 polypeptides into cells for either expression or replication thereof. Selection and use of such vectors are well known and available to the skilled artisan. In certain embodiments, such a vector includes at least one of 1) an origin of replication; 2) a selectable or detectable marker; and 3) an expression control (gene regulatory) sequence, such as a promoter.

In certain embodiments, such vectors, including expression vectors, are operatively linked with regulatory sequences, such as promoter regions, that are capable of effecting expression of heterologous DNA or RNA polynucleotide sequences encoding modified IL-2 polypeptides disclosed herein that are operatively linked to such regulatory sequences and/or promoter regions. Thus, in certain embodiments, an expression vector refers to a recombinant DNA or RNA construct, such as a plasmid, a phage, recombinant virus, or other vector that, upon introduction into an appropriate host cell, is capable of facilitating expression of a heterologous DNA or RNA polynucleotide sequence encoding such a modified IL-2 polypeptide by the host cell. Expression vectors that are suitable for use in encoding and expressing modified IL-2 polypeptides as disclosed herein are well known and available to the skilled artisan and include those that are replicable in eukaryotic cells and/or prokaryotic cells as well as those that are episomal or those which integrate into the host cell genome.

As used herein and throughout, “a promoter region or promoter element” refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated. Exemplary promoters contemplated for use in prokaryotes include the bacteriophage T7 and T3 promoters, and the like.

As used herein and throughout, “operatively linked or operationally associated” refers to the functional relationship of DNA or RNA polynucleotide sequences that encode polypeptides with regulatory and effector polynucleotide sequences. Exemplary such regulatory and/or effector polynucleotide sequences include promoters, enhancers, transcriptional and translational start sites, transcriptional and translational stop sites, polyadenylation sequences, ribosomal entry sites, Kozak sequences, and the like. For example, DNA encoding a polypeptide that is operatively linked to a promoter refers to the physical and functional relationship between the polypeptide-encoding DNA and the promoter such that the transcription of such polypeptide-encoding DNA is initiated from the promoter by an RNA polymerase that specifically recognizes, binds to and transcribes the polypeptide-encoding DNA. Additionally, RNA encoding a polypeptide that is operatively linked to a polynucleotide sequence comprising a translational start site and/or a ribosomal entry site refers to the physical and functional relationship between the polypeptide-encoding RNA and the translational start site and/or the ribosomal entry site such that the translation of the encoded polypeptide is initiated.

In order to optimize expression and/or in vitro transcription, it may be necessary to remove, add or alter 5′ untranslated portions of the certain polypeptide-encoding DNA or RNA polynucleotide sequences to eliminate extra, potentially undesirable alternative translation initiation (i.e., start) codons or other sequences that may interfere with or reduce expression, either at the level of transcription or translation. Alternatively, consensus sites can be inserted immediately 5′ of the start codon and may enhance expression. See, e.g., Kozak (1991) J. Biol. Chem. 266:19867-19870. The desirability of (or need for) such modification may be empirically determined.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent or treatment, the subject shows observable and/or measurable reduction in or absence of one or more signs and symptoms of the particular disease. Reduction of the signs or symptoms of a disease may also be felt by the patient. A patient is also considered treated if the patient experiences stable disease. In certain embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art. In certain embodiments, “treatment” means any manner in which the symptoms of a condition, disorder or disease are ameliorated or otherwise beneficially altered. Treatment also encompasses any pharmaceutical use of the compositions herein. In certain embodiments, “amelioration” of the symptoms of a particular disorder by administration of a particular pharmaceutical composition refers to any lessening, whether permanent or temporary, lasting or transient that can be attributed to or associated with administration of the composition.

The term “prediction” or “prognosis” is often used herein to refer to the likelihood that a patient will respond either favorably or unfavorably to a drug or set of drugs, or the likely outcome of a disease. In certain embodiments, the prediction relates to the extent of those responses or outcomes. In certain embodiments, the prediction relates to whether and/or the probability that a patient will survive or improve following treatment, for example treatment with a particular therapeutic agent, and for a certain period of time without disease recurrence. The predictive methods disclosed herein and throughout can be used clinically to make treatment decisions by choosing the most appropriate treatment modalities for any particular patient. The predictive methods disclosed herein and throughout are valuable tools in predicting if a patient is likely to respond favorably to a treatment regimen, such as a given therapeutic regimen, including for example, administration of a given therapeutic agent or combination, surgical intervention, steroid treatment, etc.

As used herein and throughout the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20^{th} ed., (Lippincott, Williams & Wilkins 2003). Except insofar as any conventional media or agent is incompatible with the active compound, such use in the compositions is contemplated.

A “pharmaceutically acceptable salt” is intended to mean a salt of a free acid or base of a compound represented herein and throughout that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. A modified interleukin 2 (IL-2) polypeptide or its conjugate described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

As used herein and throughout, the term “therapeutically effective amount” or “effective amount” refers to an amount of a therapeutic agent that when administered alone or in combination with an additional therapeutic agent to a cell, tissue, or subject is effective to prevent or ameliorate a disease or disorder, a proliferation disease or disorder, in a subject. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. In some embodiment, “an effective amount of a compound for treating a particular disease” is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

The term “combination” refers to either a fixed combination in one dosage unit form, or a kit of parts for the combined administration where a modified interleukin 2 (IL-2) polypeptide or its conjugate and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination”, as used herein and throughout, means a product that results from the mixing or combining of more than one active ingredient and includes both fixed and non-fixed combinations of the active ingredients. The term “fixed combination” means that the active ingredients, e.g., a modified interleukin 2 (IL-2) polypeptide or its conjugate and a combination partner, are both administered to a patient simultaneously in the form of a single entity or dosage. The term “non-fixed combination” means that the active ingredients, e.g., a modified interleukin 2 (IL-2) polypeptide or its conjugate and a combination partner, are both administered to a patient as separate entities either simultaneously, concurrently or sequentially with no specific time limits, wherein such administration provides therapeutically effective levels of the two substances in the body of the patient. The latter also applies to cocktail therapy, e.g., the administration of three or more active ingredients.

As used herein and throughout, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. In certain embodiments, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

A “healthy control” or “normal control”, in the context of therapeutic treatment or diagnostics, is a biological sample taken from an individual who does not suffer from a disease or disorder, e.g., a proliferation disease or disorder. A “negative control” is a sample that lacks any of the specific analyte the assay is designed to detect and thus provides a reference baseline for the assay.

As used herein and throughout, “mammal” refers to any of the mammalian class of species. Frequently, the term “mammal,” as used herein and throughout, refers to humans, human subjects or human patients. “Mammal” also refers to any of the non-human mammalian class of species, e.g., experimental, companion or economic non-human mammals. Exemplary non-human mammals include mice, rats, rabbits, cats, dogs, pigs, cattle, sheep, goats, horses, monkeys, gorillas and chimpanzees.

As used herein and throughout, “production by recombinant means” refers to production methods that use recombinant nucleic acid methods that rely on well-known methods of molecular biology for expressing polypeptides or proteins encoded by cloned nucleic acids.

As used herein and throughout, the term “subject” is not limited to a specific species or sample type. For example, the term “subject” may refer to a patient, and frequently a human patient. However, this term is not limited to humans and thus encompasses a variety of non-human animal or mammalian species.

As used herein and throughout, a “prodrug” is a substance that, upon in vivo administration, is metabolized or otherwise converted to the biologically, pharmaceutically or therapeutically active form of the substance. To produce a prodrug, the pharmaceutically active substance is modified such that the active substance will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is understood that aspects and embodiments disclosed herein and throughout herein include “consisting” and/or “consisting essentially of” aspects and embodiments.

Throughout this disclosure, various aspects of the disclosure are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the disclosure provided herein and throughout will become apparent from the following specification taken in conjunction with the accompanying drawings.

### Modified Interleukin 2 (IL-2) Polypeptides

In certain embodiments, the disclosure provided herein and throughout is directed to modified interleukin 2 (IL-2) polypeptides, wherein the modified interleukin 2 (IL-2) polypeptides comprise an amino acid sequence set forth in SEQ ID NO:1 (the mature form of human IL-2 comprising a cysteine to serine substitution at amino acid position 125) or SEQ ID NO:2 (the mature form of human IL-2 comprising a alanine to methionine substitution at amino acid position 1), or an amino acid having at least 80% identity to either SEQ ID NO:1 or SEQ ID NO:2, where the modified IL-2 polypeptide includes a substitution with a natural or unnatural amino acid at amino acid position L18, L19, N29, N30, Y31, V69, N71, Q74, N88, V91, E100, N119, T123, S127, I128, T131, or a combination thereof, wherein the modified IL-2 polypeptide: a)) has reduced binding to an interleukin 2 receptor α (IL-2Rα) compared to a comparable IL-2 polypeptide without the amino acid substitution at L18, L19, N29, N30, Y31, V69, N71, Q74, N88, V91, E100, N119, T123, S127, I128, T131, or a combination thereof; and/or b) has reduced receptor signaling potency to IL-2Rαβγ compared to a comparable IL-2 polypeptide, where a comparable IL-2 polypeptide is identical in sequence to the modified IL-2 polypeptide with the exception that the comparable IL-2 polypeptide does not include the amino acid substitution of the modified IL-2 polypeptide (e.g., an amino acid substitution at L18, L19, N29, N30, Y31, V69, N71, Q74, N88, V91, E100, N119, T123, S127, I128, T131, or a combination thereof). In various embodiments, such modifications are found within an IL-2Rα interaction region, an IL-2Rβ interaction region and/or an IL-2Rγ interaction region. In certain embodiments, modified IL-2 polypeptides have at least about 70% sequence identity, at least about 80% sequence identity, at least about 90% sequence identity, at least about 95% sequence identity, at least about 98% sequence identity, or at least about 99% sequence identity, independently in the region of amino acid residues 10-25, 80-100 and/or 100-134 of the corresponding region of an IL-2 polypeptide comprising an amino acid sequence set forth in SEQ ID NO:1 or SEQ ID NO:2 without the substitution. In certain embodiments, modified IL-2 polypeptides have at least about 50% sequence identity, at least about 90% sequence identity, at least about 70% sequence identity, at least about 80% sequence identity, at least about 90% sequence identity, at least about 95% sequence identity, at least about 98% sequence identity, or at least about 99% sequence identity to an IL-2 polypeptide comprising an amino acid sequence set forth in SEQ ID NO:1 or SEQ ID NO:2 without the substitution.

The amino acid sequences of SEQ ID NO:1 or SEQ ID NO:2 are set forth below:

SEQ ID NO: 1
(¹APTSSSTKKTQL¹³QLEHLL¹⁹LDLQMILNGI²⁹N³⁰N³¹Y³²K³³N
³⁴P³⁵KLT³⁸RML⁴¹T⁴²F⁴³KF⁴⁵YMP⁴⁸K49KATELKHLQCLEE⁶²EL
⁶⁴K⁶⁵PLEEVL⁷¹NLA⁷⁴QS⁷⁶KNFHL⁸¹RPRD⁸⁵LI⁸⁷SNIN⁹¹V⁹²I
⁹³VLELKGSETTFMCEYADETATIVEFLNRWITFSQSIISTL¹³³T)
SEQ ID NO: 2
(¹MPTSSSTKKTQL¹³QLEHLL¹⁹LDLQMILNGI²⁹N³⁰N³¹Y³²K³³N
³⁴P³⁵KLT³⁸RML⁴¹T⁴²F⁴³KF⁴⁵YMP⁴⁸K49KATELKHLQCLEE⁶²EL
⁶⁴K⁶⁵PLEEVL⁷¹NLA⁷⁴QS⁷⁶KNFHL⁸¹RPRD⁸⁵LI⁸⁷SNIN⁹¹V⁹²I
⁹³VLELKGSETTFMCEYADETATIVEFLNRWITFCQSIISTL¹³³T).

SEQ ID NO:1 and SEQ ID NO:2 differ from each other and from the native human IL-2 sequence (SEQ ID NO:3) in the identities of amino acids at position 1 (where SEQ ID NO:2 differs from the native human sequence and from SEQ ID NO:1) and position 125 (where SEQ ID NO:1 differs from the native human sequence and from SEQ ID NO:2). Thus the amino acid numbering used in the disclosure provided herein and throughout follows that of the native human mature IL-2 amino acid sequence (SEQ ID NO:3), SEQ ID NO:1, and SEQ ID NO:2.

A modified IL-2 polypeptide as provided herein has at least about 80% sequence identity, e.g., at least about at least about 80%, 81%, 82%, 83%, 84%, 85%, 86%, 87%, 88%, 89%, 90%, 91%, 92%, 93%, 94%, 95%, 96%, 97%, 98%, or 99% sequence identity or more to the amino acid sequence set forth in SEQ ID NO:1 or SEQ ID NO:2 without the substitution. In certain embodiments, a modified IL-2 polypeptide as provided herein has a sequence identical to that of SEQ ID NO:1 or SEQ ID NO:2 with the exception of the substituted amino acids at any of positions L18, L19, N29, N30, Y31, V69, N71, Q74, N88, V91, E100, N119, T123, S127, I128, T131, including any combination thereof. An IL-2 polypeptide that does not include the one or more amino acid substitutions of a modified IL-2 polypeptide as provided herein (e.g., a substitution with respect to SEQ ID NO:1 or SEQ ID NO:2 at any of amino acid positions L18, L19, N29, N30, Y31, V69, N71, Q74, N88, V91, E100, N119, T123, S127, I128, T131, or any combination thereof) but otherwise has a sequence identical to the sequence of the modified IL-2 polypeptide provided herein is referred to herein as a “comparable IL-2 polypeptide that does not have the [amino acid position(s)] substitution(s)”, “an IL-2 polypeptide that does not have the [amino acid position(s)] substitution(s)”, or similar phraseology that provides the same meaning. One of skill in the art can be guided in making substitutions outside of the disclosed mutation sites to maintain active IL-2 polypeptides by knowledge of neutral and conservative amino acid changes and regions of the IL-2 polypeptide less likely to be tolerant of mutations.

In certain embodiments, a modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity, and preferably at least 90%, 91%, 92%, 93%, 94%, 95%, 96%, 97%, 98%, 99% or 100% sequence identity, or at least 95% sequence identity, in the region of amino acid residues 10-25 to the corresponding region of a comparable IL-2 polypeptide comprising an amino acid sequence set forth in SEQ ID NO:1 or SEQ ID NO:2 without a substitution at any of the aforementioned amino acid positions.

In other embodiments, a modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity, and preferably at least 90%, 91%, 92%, 93%, 94%, 95%, 96%, 97%, 98%, 99% or 100% sequence identity, or at least 95% sequence identity in the region of amino acid residues 80-100 to the corresponding region of a comparable IL-2 polypeptide comprising an amino acid sequence set forth in SEQ ID NO:1 or SEQ ID NO:2 without the substitution.

In still other embodiments, a modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100%, and preferably at least 90%, 91%, 92%, 93%, 94%, 95%, 96%, 97%, 98%, 99% or 100% sequence identity, or at least 95% sequence identity in the region of amino acid residues 100-133 to the corresponding region of a comparable IL-2 polypeptide comprising an amino acid sequence set forth in SEQ ID NO:1 or SEQ ID NO:2 without the substitution. In certain embodiments, a modified IL-2 polypeptide includes the mutation C125S (e.g., SEQ ID NO:1). Other C125 substitutions can be present in a modified IL-2 polypeptide as provided herein, such as, as nonlimiting examples, the mutations C125A and C125T.

In additional embodiments, a modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100%, and preferably at least 90%, 91%, 92%, 93%, 94%, 95%, 96%, 97%, 98%, 99% or 100% sequence identity, or at least 95% sequence identity in the regions of amino acid residues 10-25 and 80-100 to the corresponding regions of a comparable IL-2 polypeptide comprising an amino acid sequence set forth in SEQ ID NO:1 or SEQ ID NO:2 without the substitution.

In yet another embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity, and preferably at least 90%, 91%, 92%, 93%, 94%, 95%, 96%, 97%, 98%, 99% or 100% sequence identity, or at least 95% sequence identity, in the regions of amino acid residues 10-25 and 100-133 to the corresponding regions of a comparable IL-2 polypeptide comprising an amino acid sequence set forth in SEQ ID NO:1 or SEQ ID NO:2 without the substitution.

In yet another embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity, and preferably at least 90%, 91%, 92%, 93%, 94%, 95%, 96%, 97%, 98%, 99% or 100% sequence identity, or at least 95% sequence identity, in the regions of amino acid residues 80-100 and 100-133 to the corresponding regions of a comparable IL-2 polypeptide comprising an amino acid sequence set forth in SEQ ID NO:1 or SEQ ID NO:2 without the substitution.

In yet another embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity, and preferably at least 90%, 91%, 92%, 93%, 94%, 95%, 96%, 97%, 98%, 99% or 100% sequence identity, or at least 95% sequence identity, in the regions of amino acid residues 10-25, 80-100 and 100-133 to the corresponding regions of a comparable IL-2 polypeptide comprising an amino acid sequence set forth in SEQ ID NO:1 or SEQ ID NO:2 without the substitution.

In certain embodiments, modified IL-2 polypeptides can comprise any suitable substitution with a natural amino acid. In certain embodiments, the present modified IL-2 polypeptide can comprise a substitution with lysine, cysteine, serine, histidine, methionine, arginine, aspartic acid, glutamic acid, threonine, alanine, tryptophan, isoleucine, phenylalanine, or tyrosine at a position of L18, L19, N29, Y31, V69, N71, Q74, N88, V91, E100, N119, T123, S127, I128, T131, or a combination thereof.

In certain embodiments, a modified IL-2 polypeptide as provided herein: a) comprises a substitution with a natural amino acid at a position selected from the group consisting of L18, L19, N29, Y31, V69, N71, Q74, N88, V91, E100, N119, T123, S127, I128, T131, and a combination thereof, and is conjugated to, or configured to be conjugated to, a conjugating moiety. In certain embodiments, such conjugating moieties may comprise a water-soluble a polymer, a lipid, a protein, or a peptide, of combinations thereof. In certain embodiments, modified IL-2 polypeptides comprise a) one or more conjugating moieties, such as a water-soluble a polymer, a lipid, a protein, or a peptide, of combinations thereof, at one or more positions selected from the group consisting of L18, L19, N29, Y31, V69, N71, Q74, N88, V91, E100, N119, T123, S127, I128, T131, and combinations thereof; and/or b) comprise a substitution with a natural amino acid at a position selected from the group consisting of L18, L19, N29, Y31, V69, N71, Q74, N88, V91, E100, N119, T123, S127, I128, T131, and combinations thereof, and are conjugated to, or configured to be conjugated to, one or more conjugating moieties, such as a water-soluble polymer, a lipid, a protein, or a peptide, at the N terminal and/or C terminal of the polypeptide.

In certain embodiments, modified IL-2 polypeptides disclosed herein and throughout: a) comprise a substitution with natural amino acid at a position selected from the group consisting of N29 and/or Y31; b) comprise a substitution with cysteine or serine at a position selected from the group consisting of N29 and/or Y31 and a combination thereof; c) comprise a substitution with cysteine at a position selected from the group consisting of N29, and/or Y31 and a combination thereof; d) comprise a substitution with cysteine, at a position Y31, e) comprise a substitution with serine at a position Y31, f) comprises a substitution with cysteine, at a position N29, or g) comprise a substitution with serine at a position N29.

In certain embodiments, modified IL-2 polypeptides disclosed herein and throughout that include a substitution with a natural amino acid at N29 or Y31 can further comprise: a) a substitution with a natural amino acid at L18 and/or L19; and b) a substitution with methionine at a position L18, and/or a substitution with serine at a position L19.

In certain embodiments, modified IL-2 polypeptides disclosed herein and throughout can further comprise: a) a substitution with a natural amino acid at a position within IL-2Rα interaction region and a substitution with a natural amino acid at a position within IL-2Rβ interaction region; b) a substitution with a natural amino acid at a position within IL-2Rα interaction region and a substitution with a natural amino acid at a position within IL-2Rγ interaction region; or c) a substitution with a natural amino acid at a position within IL-2Rα interaction region, a substitution with a natural amino acid at a position within IL-2Rβ interaction region and a substitution with a natural amino acid at a position within IL-2Rγ interaction region.

In certain embodiments, modified IL-2 polypeptides disclosed herein and throughout, in addition to a mutation at N29 or Y31 and optionally a mutation at L18 and/or L19, can further comprise a substitution with a natural amino acid or an unnatural amino acid at a position within IL-2Rα interaction region, IL-2Rβ interaction region and/or IL-2Rγ interaction region. In certain embodiments, modified IL-2 polypeptides disclosed herein and throughout can further comprise a substitution with natural amino acids at a position selected from the group consisting of V69, N71, Q74, N88, V91, I128, and a combination thereof.

In certain embodiments, modified IL-2 polypeptides disclosed herein and throughout may, alternatively, or in addition to, having a mutation at N29 or Y31, and optionally one or more additional mutations at any of V69, N71, Q74, N88, V91, I128, can further comprise a substitution with a natural amino acid or an unnatural amino acid at a position selected from E100, N119, T123, S127, I128, T131, and a combination thereof. In certain embodiments, modified IL-2 polypeptides disclosed herein and throughout can further comprise a substitution with lysine, cysteine, histidine, at a position selected from E100, N119, T123, S127, I128, T131, and a combination thereof.

In certain embodiments, modified IL-2 polypeptides disclosed herein and throughout can comprise any suitable substitution with an unnatural amino acid. In certain embodiments, the unnatural amino acids disclosed in WO 2019/028425 A1 and WO 2019/028419 A1 can be used. In certain embodiments, modified IL-2 polypeptides disclosed herein and throughout, an unnatural amino acid can be a lysine analogue, a cysteine analogue or a histidine analogue, comprises an aromatic side chain; comprises an azido group; comprises an alkyne group; or comprises an aldehyde or ketone group. In another embodiment, the unnatural amino acid does not comprise an aromatic side chain. In still another embodiment, the unnatural amino acid comprises N6-azidoethoxy-L-lysine (AzK), N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, 0-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, or selenocysteine.

In certain embodiments disclosed herein and throughout, the unnatural amino acid can be incorporated into the modified IL-2 polypeptide by any suitable means or methods. In certain embodiments disclosed herein and throughout, the unnatural amino acid can be incorporated into the modified IL-2 polypeptide by an orthogonal tRNA synthetase/tRNA pair. Any suitable orthogonal tRNA can be used. In certain embodiments disclosed herein and throughout, the orthogonal tRNA of the orthogonal synthetase/tRNA pair can comprise at least one unnatural nucleobase.

In certain embodiments, modified IL-2 polypeptides disclosed herein and throughout can have enhanced binding to an IL-2Rα compared to a comparable IL-2 polypeptide without the amino acid substitution. In certain embodiments disclosed herein and throughout, the binding affinity of the present modified IL-2 polypeptide to an IL-2Rα can be increased from about 10% to about 100%, or a subrange thereof, e.g., can be increased by about 10%, 20%, 30%, 40%, 50%, 60%, 70%, 80%, 90%, 100%. In certain embodiments disclosed herein and throughout, the binding affinity of the present modified IL-2 polypeptide to an IL-2Rα can be increased from about 10% to about 100%, or can be increased from about 1-fold to about 100,000-fold or more, e.g., increased by at least 1-fold (100%), 10-fold, 100-fold, 1,000-fold, 10,000-fold, 100,000-fold or more, e.g., between about 1-fold and 100,000-fold, or a subrange thereof.

In certain embodiments modified IL-2 polypeptides disclosed herein and throughout can have increased receptor signaling potency to IL-2Rαβγ compared to a comparable IL-2 polypeptide without the substitution. In certain embodiments disclosed herein and throughout, the signaling potency to IL-2Rαβγ of the present modified IL-2 polypeptide and the signaling potency to IL-2Rαβγ of the comparable IL-2 polypeptide without the substitution can be increased from about 10% to about 100%, or a subrange thereof, e.g., can be increased by about 10%, 20%, 30%, 40%, 50%, 60%, 70%, 80%, 90%, 100%. In certain embodiments disclosed herein and throughout, the signaling potency of the present modified IL-2 polypeptide to an IL-2Rαβγ can be increased from about 10% to about 100%, or can be increased from about 1-fold to about 100,000-fold or more, e.g., increased by about 1-fold (100%), 10-fold, 100-fold, 1,000-fold, 10,000-fold, 100,000-fold or more, or by between about 1-fold and 100,000-fold or a subrange thereof.

In certain embodiments modified IL-2 polypeptides disclosed herein and throughout can have reduced internalization by IL-2Rα and/or IL-2Rαβγ expressing cells compared to a comparable IL-2 polypeptide without the amino acid substitution. In certain embodiments, the ratio between the internalization by IL-2Rα and/or IL-2Rαβγ expressing cells of the present modified IL-2 polypeptide and the internalization by IL-2Rα and/or IL-2Rαβγ expressing cells of the comparable IL-2 polypeptide without the substitution can be from about 1/2 to about 1/100,000, e.g., at about 1/2, 1/5, 1/10, 1/100, 1/1,000, 1/10,000, 1/100,000, or more, or a subrange thereof. In certain embodiments, a modified IL-2 polypeptide has no detectable internalization by IL-2Rα and/or IL-2Rαβγ expressing cells.

In certain embodiments modified IL-2 polypeptides disclosed herein and throughout can have an enhanced ratio of IL-2Rαβγ/IL-2Rβγ signaling potency as compared to the ratio of IL-2Rαβγ/IL-2Rβγ signaling potency of a comparable IL-2 polypeptide without the substitution. In another embodiment, the ratio of IL-2Rαβγ/IL-2Rβγ r signaling potency of a modified IL-2 polypeptide as disclosed herein compared to the ratio of IL-2Rαβγ/IL-2Rβγ signaling potency to of a comparable IL-2 polypeptide without the substitution of the modified IL-2 polypeptide can be increased by 10%, 20%, 30%, 40%, 50%, 60%, 70%, 80%, 90%, 100%, or greater than 100%, for example increased by between about 10% and about 100% or a subrange thereof. In further embodiments, the ratio of IL-2Rαβγ/IL-2Rβγ signaling potency of a modified IL-2 polypeptide compared to the ratio of IL-2Rαβγ/IL-2Rβγ signaling potency to of a comparable IL-2 polypeptide without the substitution can be increased from about 10% to about 100%, or can be increased from about 1-fold (100%) to about 100,000-fold or a subrange thereof, or more, e.g., increased by about 1-fold, 10-fold, 100-fold, 1,000-fold, 10,000-fold, 100,000-fold or more.

I In certain embodiments modified IL-2 polypeptides disclosed herein and throughout can have the sequence of SEQ ID NO:1 or SEQ ID NO:2 with the exception of one or more of the amino acid substitutions disclosed hereinabove, such as one or more amino acids substitutions of the group L18, L19, N29, Y31, V69, N71, Q74, N88, V91, E100, N119, T123, S127, I128, and T131. In such examples, a comparable IL-2 polypeptide has the sequence of SEQ ID NO:1 or SEQ ID NO:2. In further examples, a modified IL-2 polypeptide having one or more of the amino acid substitutions disclosed hereinabove, such as one or more amino acids substitutions at amino acid positions selected from the group L18, L19, N29, Y31, V69, N71, Q74, N88, V91, E100, N119, T123, S127, I128, and T131, can have a sequence having at least 80%, at least 85%, at least 90%, at least 95%, at least 96%, at least 97%, at least 98% identity to SEQ ID NO:1 or SEQ ID NO:2, where a comparable IL-2 polypeptide has an identical sequence to the modified IL-2 polypeptide, with the exception of the one or more amino acids substitutions at positions selected from the group L18, L19, N29, Y31, V69, N71, Q74, N88, V91, E100, N119, T123, S127, I128, and T131.

In certain embodiments modified IL-2 polypeptides disclosed herein and throughout can comprise a deletion at any suitable location. In certain embodiments, the present modified IL-2 polypeptide has a N terminal deletion, e.g., a N terminal deletion of amino acid residues 1-10 or a subrange thereof. In another embodiment, the present modified IL-2 polypeptide has a C terminal deletion, e.g., a C terminal deletion of amino acid residues 114-133 or a subrange thereof. In still another embodiment, the present modified IL-2 polypeptide has a N terminal deletion and a C terminal deletion.

In certain embodiments modified IL-2 polypeptides disclosed herein and throughout can be a part of a fusion polypeptide, e.g., a recombinant fusion protein, that comprises the modified IL-2 polypeptide and an additional amino acid sequence. Such modified IL-2 polypeptides that are part of a fusion polypeptide are, themselves examples of modified IL-2 polypeptides. The present modified IL-2 polypeptide can be fused to the additional amino acid sequence in any suitable manner. In certain embodiments, the N terminus or the C terminus of the modified IL-2 polypeptide can be fused to the additional amino acid sequence or vice versa. The additional amino acid sequence can comprise any suitable sequence or content. In certain embodiments, the additional amino acid sequence can comprise an antibody sequence or a portion or a fragment thereof. In another example, the additional amino acid sequence can comprise a Fc portion of an antibody.

In certain embodiments modified IL-2 polypeptides disclosed herein and throughout can be in any suitable form. In certain embodiments modified IL-2 polypeptides disclosed herein and throughout can be in an isolated or purified form.

In certain embodiments modified IL-2 polypeptides disclosed herein and throughout can be prepared using any suitable technique or process. In certain embodiments, a modified IL-2 polypeptide as provided herein can be prepared by recombinant production, chemical synthesis, or a combination thereof.

In certain embodiments modified IL-2 polypeptides disclosed herein and throughout can be applied in any suitable form. In certain embodiments modified IL-2 polypeptides disclosed herein and throughout can be delivered, e.g., administered to a patient, by administering an RNA polynucleotide, a DNA polynucleotide that encodes the modified IL-2 polypeptide. In certain embodiments modified IL-2 polypeptides disclosed herein and throughout can be delivered, e.g., administered to a patient, by administering an non-viral vector or a viral vector that encodes the modified IL-2 polypeptide.

### Modified Interleukin 2 (IL-2) Polypeptide Conjugates

In certain embodiments, the disclosure provided herein and throughout is directed to modified IL-2 polypeptide conjugate, which comprises a modified IL-2 polypeptide, as disclosed herein and throughout. Such modified IL-2 polypeptide conjugates, which are, themselves, exemplary modified IL-2 polypeptides, may be conjugated to, or configured to be conjugated to, conjugating moieties such as, e.g., water-soluble polymers, lipids, polypeptides, proteins, peptides, and combinations thereof.

The modified IL-2 polypeptide can be conjugated to another moiety, e.g., a water-soluble polymer, a lipid, a protein, or a peptide, in any suitable manner. In certain embodiments, the modified IL-2 polypeptide can be conjugated to a water-soluble polymer, a lipid, a protein, or a peptide covalently. In another example, the modified IL-2 polypeptide can be conjugated to a water-soluble polymer, a lipid, a protein, or a peptide non-covalently. In still another example, the modified IL-2 polypeptide can be conjugated to a water-soluble polymer, a lipid, a protein, or a peptide via a substituted natural amino acid or unnatural amino acid at any suitable position.

In certain embodiments modified IL-2 polypeptides disclosed herein and throughout are conjugated to another moiety, e.g., a water-soluble polymer, a lipid, a protein, or a peptide, via a substituted natural amino acid or unnatural amino acid at a position selected from the group consisting of N29, N30, Y31, E100, N119, T123, S127, I128, T131 and a combination thereof. In another embodiment, the modified IL-2 polypeptide is conjugated to another moiety, e.g., a water-soluble polymer, a lipid, a protein, or a peptide, via a substituted natural amino acid at a position selected from the group consisting of N29, N30, Y31, E100, N119, T123, S127, I128, T131 and a combination thereof. In still another embodiment, the modified IL-2 polypeptide is conjugated to another moiety, e.g., a water-soluble polymer, a lipid, a protein, or a peptide, via a substituted lysine, cysteine, histidine, serine, threonine, tryptophan, glutamine, asparagine, arginine, proline, phenylalanine, or tyrosine at a position selected from the group consisting of N29, N30, Y31, E100, N119, T123, S127, I128, T131 and a combination thereof. In yet another embodiment, the modified IL-2 polypeptide is conjugated to another moiety, e.g., a water-soluble polymer, a lipid, a protein, or a peptide, via a substituted cysteine at a position selected from the group consisting of N29, N30, Y31, E100, N119, T123, S127, I128, T131 and a combination thereof.

The modified IL-2 polypeptide can be conjugated to another moiety, e.g., a water-soluble polymer, a lipid, a protein, or a peptide, via a substituted natural amino acid or unnatural amino acid at a position selected from the group consisting of N29, N30, Y31, and a combination thereof. In certain embodiments, the modified IL-2 polypeptide is conjugated to another moiety, e.g., a water-soluble polymer, a lipid, a protein, or a peptide, via a substituted natural amino acid at the position N29 and/or the position Y31. In another embodiment, the modified IL-2 polypeptide is conjugated to another moiety, e.g., a water-soluble polymer, a lipid, a protein, or a peptide, via a substituted lysine, cysteine, histidine, serine, threonine, tryptophan, glutamine, asparagine, arginine, proline, phenylalanine, or tyrosine at one or both of N29 and Y31. In still another embodiment, the modified IL-2 polypeptide is conjugated to another moiety, e.g., a water-soluble polymer, a lipid, a protein, or a peptide, via a substituted cysteine at one or both of N29 and Y31.

The modified IL-2 polypeptide can be conjugated to another moiety, e.g., a water-soluble polymer, a lipid, a protein, or a peptide, via a single amino acid residue or multiple amino acid residues of the modified IL-2 polypeptide. In certain embodiments, the modified IL-2 polypeptide can be conjugated to another moiety, e.g., a water-soluble polymer, a lipid, a protein, or a peptide, via: i) the alpha amino group of the N-terminal amino acid residue of the modified IL-2 polypeptide; ii) the epsilon amino group of a lysine amino acid residue of the modified IL-2 polypeptide; or iii) an N-glycosylation site or O-glycosylation site of the modified IL-2 polypeptide.

The modified IL-2 polypeptide can be covalently conjugated to another moiety, e.g., a water-soluble polymer, a lipid, a protein, or a peptide, through a linker. The modified IL-2 polypeptide can also be covalently conjugated to another moiety, e.g., a water-soluble polymer, a lipid, a protein, or a peptide, directly without a linker.

The modified IL-2 polypeptide can be conjugated to another moiety, e.g., a water-soluble polymer, a lipid, a protein, or a peptide, via a single amino acid residue in a fusion polypeptide that comprises the modified IL-2 polypeptide and an additional amino acid sequence. The single amino acid residue can be located at any suitable location. In certain embodiments, the single amino acid residue can be located within the modified IL-2 polypeptide. In another example, the single amino acid residue can be located within the additional amino acid sequence.

The additional amino acid sequence in the present modified IL-2 polypeptide conjugate can comprise any suitable sequence or content. In certain embodiments, the additional amino acid sequence in the present modified IL-2 polypeptide conjugate can comprise an antibody sequence or a portion or a fragment thereof. In another example, the additional amino acid sequence in the present modified IL-2 polypeptide conjugate can comprise a Fc portion of an antibody.

The modified IL-2 polypeptide can be conjugated to another moiety, e.g., a water-soluble polymer, a lipid, a protein, or a peptide in a fusion polypeptide, in any suitable manner. In certain embodiments, the modified IL-2 polypeptide can be conjugated to another moiety, e.g., a water-soluble polymer, a lipid, a protein, or a peptide, via: i) the alpha amino group of the N-terminal amino acid residue of the fusion polypeptide; ii) the epsilon amino group of a lysine amino acid residue of the fusion polypeptide; or iii) an N-glycosylation site or O-glycosylation site of the fusion polypeptide. In another example, the fusion polypeptide can be covalently conjugated to a water-soluble polymer, a lipid, a protein, or a peptide directly or through a linker.

The present modified IL-2 polypeptide can be conjugated to any suitable water-soluble polymer. In certain embodiments, the water-soluble polymer can comprise polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof. See e.g., WO 2019/028425A1 and WO 2019/028419A1.

In the present modified IL-2 polypeptide conjugate, the water-soluble polymer can comprise a PEG molecule. The PEG molecule can be a linear PEG or a branched PEG. The branched PEG can have any suitable configuration and/or any suitable number of PEG chains. In certain embodiments, the branched PEG can have about three to about ten PEG chains emanating from a central core group. In another example, the branched PEG can be a star PEG comprising from about 10 to about 100 PEG chains emanating from a central core group. In still another example, the branched PEG can be a comb PEG comprising multiple PEG chains grafted onto a polymer backbone.

The PEG molecule in the present modified IL-2 polypeptide conjugate can have any suitable molecular weight. In certain embodiments, the PEG molecule can have a range of molecular weight from about 300 g/mol to about 10,000,000 g/mol, e.g., at about 300 g/mol, 500 g/mol, 1,000 g/mol, 10,000 g/mol, 100,000 g/mol, 1,000,000 g/mol, 10,000,000 g/mol or a subrange thereof. In another example, the PEG molecule can have an average molecular weight from about 5,000 Daltons to about 1,000,000 Daltons, e.g., at about 5,000 Daltons, 10,000 Daltons, 100,000 Daltons, 1,000,000 Daltons or a subrange thereof. In still another example, the PEG molecule can have an average molecular weight of from about 20,000 Daltons to about 30,000 Daltons, e.g., at about 20,000 Daltons, 21,000 Daltons, 22,000 Daltons, 23,000 Daltons, 24,000 Daltons, 25,000 Daltons, 26,000 Daltons, 27,000 Daltons, 28,000 Daltons, 29,000 Daltons, 30,000 Daltons or a subrange thereof.

The PEG molecule in the present modified IL-2 polypeptide conjugate can be in any suitable form. In certain embodiments, the PEG molecule can be a monodisperse, uniform, or discrete PEG molecule.

The water-soluble polymer in the present modified IL-2 polypeptide conjugate can comprise a polysaccharide.

The modified IL-2 polypeptide in the present modified IL-2 polypeptide conjugate can be conjugated to any suitable lipid. In certain embodiments, the lipid in the present modified IL-2 polypeptide conjugate can comprise a fatty acid.

The modified IL-2 polypeptide in the present modified IL-2 polypeptide conjugate can be conjugated to any suitable protein. In certain embodiments, the protein in the present modified IL-2 polypeptide conjugate can comprise an antibody or a binding fragment thereof. The antibody or a binding fragment thereof can comprise an Fc portion of an antibody.

In the present modified IL-2 polypeptide conjugate, the other moiety, e.g., a water-soluble polymer, a lipid, a protein, or a peptide, can be bound to the modified IL-2 polypeptide via any suitable manner. In certain embodiments, the other moiety, e.g., a water-soluble polymer, a lipid, a protein, or a peptide, can be indirectly bound to the substituted natural amino acid or unnatural amino acid of the modified IL-2 polypeptide through a linker. In another example, the other moiety, e.g., a water-soluble polymer, a lipid, a protein, or a peptide, can be directly bound to the substituted natural amino acid or unnatural amino acid of the modified IL-2 polypeptide.

The present modified IL-2 polypeptide conjugate can have any suitable half-life in vivo. In certain embodiments, the present modified IL-2 polypeptide conjugate can have a half-life in vivo from about 5 minutes to about 10 days, e.g., at about 5 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hour, 12 hours, 13 hours, 14 hours, 15 hours, 16 hours, 17 hours, 18 hours, 19 hours, 20 hours, 21 hour, 22 hours, 23 hours, 1 day, 2 days, 3 days, 4 days, 5 days, 6 days, 7 days, 8 days, 9 days, 10 days or a subrange thereof.

### Pharmaceutical Compositions

In certain embodiments, the disclosure provided herein and throughout is directed to a pharmaceutical composition comprising an effective amount of a modified IL-2 polypeptide or a modified IL-2 polypeptide conjugate, as described above, and a pharmaceutically acceptable carrier or excipient.

In certain embodiments, pharmaceutical compositions can be configured to treat or prevent any suitable disease(s), disorder(s), or condition(s). In certain embodiments, the present pharmaceutical composition can be configured to treat or prevent a proliferation disorder in a subject.

In certain embodiments, the present pharmaceutical composition is configured to treat or prevent a solid tumor or cancer in a subject. The solid tumor or cancer can be Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor or Liposarcoma.

In another embodiment, the present pharmaceutical composition is configured to treat or prevent a hematological malignancy in a subject. The hematological malignancy can be hematological malignancy including: myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenström's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schöner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenström's macroglobulinaemia, Alpha heavy chain disease, Gamma heavy chain disease, Franklin's disease, Immunoproliferative small intestinal disease, Mediterranean disease, Malignant immunoproliferative disease, unspecified, or Immunoproliferative disease NOS.

In still another embodiment, the present pharmaceutical composition is configured to treat or prevent an immune deficiency disease or disorder in a subject. The immune deficiency disease or disorder can be: Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs) or Lymphopenia.

In still another embodiment, the present pharmaceutical composition is configured to treat or prevent an inflammatory or autoimmune diseases in a subject. The inflammatory or autoimmune diseases can be: inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, and transplantation rejection.

In still another embodiment, the present pharmaceutical composition is configured to treat or prevent an infectious diseases or disorder in a subject. Infectious diseases or disorder can be: Acinetobacter infections, Actinomycosis, African sleeping sickness (African trypanosomiasis), AIDS (acquired immunodeficiency syndrome), Amoebiasis, Anaplasmosis, Angiostrongyliasis Anisakiasis, Anthrax, Arcanobacterium haemolyticum infection, Argentine hemorrhagic fever, Ascariasis, Aspergillosis Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus (children and babies), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, SARS (severe acute respiratory syndrome), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, and Zygomycosis.

The present pharmaceutical composition can further comprise another active ingredient. The other active ingredient can the active ingredient to treat or prevent any suitable any suitable disease(s), disorder(s) or condition(s). In certain embodiments, the other active ingredient can be an anti-neoplasm substance.

The additional active ingredient(s) may be formulated in a separate pharmaceutical composition from at least one exemplary modified IL-2 polypeptide or modified IL-2 polypeptide conjugate of the present disclosure or may be included with at least one exemplary modified IL-2 polypeptide or modified IL-2 polypeptide conjugate of the present disclosure in a single pharmaceutical composition.

The present pharmaceutical compositions can be formulated to be administered orally, parenterally, by inhalation, topically, rectally, nasally, buccally, vaginally, via an implanted reservoir, or other drug administration methods. The term “parenteral” as used herein and throughout includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

Any suitable formulation of the compounds described herein can be prepared. See generally, Remington's Pharmaceutical Sciences, (2000) Hoover, J. E. editor, 20 th edition, Lippincott Williams and Wilkins Publishing Company, Easton, Pa., pages 780-857. A formulation is selected to be suitable for an appropriate route of administration. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made. Where contemplated compounds or substances are administered in a pharmacological composition, it is contemplated that the compounds or substances can be formulated in admixture with a pharmaceutically acceptable excipient and/or carrier. In certain embodiments, contemplated compounds or substances can be administered orally as neutral compounds or substances or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds or substances may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound or substance in order to manage the pharmacokinetics of the present compounds or substances, e.g., the present modified IL-2 polypeptide(s) or modified IL-2 polypeptide conjugate(s), for maximum beneficial effect in a patient.

The present modified IL-2 polypeptide or modified IL-2 polypeptide conjugate may be soluble in organic solvents such as chloroform, dichloromethane, ethyl acetate, ethanol, methanol, isopropanol, acetonitrile, glycerol, N,N-dimethylformamide, N,N-dimetheylaceatmide, dimethylsulfoxide, etc. In certain embodiments, the disclosure provided herein and throughout provides formulations prepared by mixing the present modified IL-2 polypeptide or modified IL-2 polypeptide conjugate with a pharmaceutically acceptable carrier. In certain embodiments, the formulation may be prepared using a method comprising: a) dissolving a described compound or substance in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In certain embodiments, the carbohydrate comprises dextrose. The pharmaceutical compositions obtained using the present methods are stable and useful for animal and clinical applications.

Illustrative examples of water soluble organic solvents for use in the present pharmaceutical compositions include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present pharmaceutical compositions include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present pharmaceutical compositions include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present pharmaceutical compositions include but are not limited to oleic acid, monoglycerides, diglycerides, a mono- or di-fatty acid ester of PEG, or a combination thereof.

Illustrative examples of cyclodextrins for use in the present pharmaceutical compositions include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present pharmaceutical compositions include but are not limited to soy phosphatidylcholine, or distearoyl phosphatidylglycerol, and hydrogenated forms thereof, or a combination thereof.

One of ordinary skill in the art may modify the formulations within the teachings of the specification to provide numerous formulations for a particular route of administration. In particular, the compounds or substances may be modified to render them more soluble in water or other vehicle. It is also well within the ordinary skill of the art to modify the route of administration and dosage regimen of a particular compound or substance in order to manage the pharmacokinetics of the present compounds or substances for maximum beneficial effect in a patient.

### Methods for Treating or Preventing a Disease or a Disorder

In certain embodiments, the disclosure provided herein and throughout is directed to a method for treating or preventing a disease or a disorder, e.g., a proliferation disease or disorder, in a subject in need comprising administering to said subject an effective amount of a modified IL-2 polypeptide, a modified IL-2 polypeptide conjugate or a pharmaceutical composition, as described above.

In certain embodiments, the methods disclosed herein and throughout can be used for treating or preventing a disease or a disorder, e.g., a proliferation disease or disorder, in any suitable subject. In certain embodiments, the methods disclosed herein and throughout can be used for treating or preventing a disease or a disorder, e.g., a proliferation disease or disorder, in a human. In certain embodiments, the methods disclosed herein and throughout can be used for treating or preventing a disease or a disorder, e.g., a proliferation disease or disorder, in a non-human mammal.

In certain embodiments, the methods disclosed herein and throughout can be used to treat a proliferation disorder in a subject. In certain embodiments, the methods disclosed herein and throughout can be used to prevent a proliferation disorder in a subject.

In certain embodiments, the methods disclosed herein and throughout can be used for treating or preventing any suitable proliferation disease or disorder in a subject. In certain embodiments, the methods disclosed herein and throughout can be used for treating or preventing a tumor in a subject. In certain embodiments, the methods disclosed herein and throughout can be used for treating or preventing a cancer in a subject.

In certain embodiments, the methods disclosed herein and throughout can be used to treat or prevent a solid tumor or cancer in a subject. In certain embodiments, the methods disclosed herein and throughout can be used to treat or prevent any suitable solid tumor or cancer in a subject. In certain embodiments, the methods disclosed herein and throughout the solid tumor or cancer can be Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor or Liposarcoma.

In certain embodiments, the methods disclosed herein and throughout can be used to treat or prevent a hematological malignancy in a subject. In certain embodiments, the methods disclosed herein and throughout can be used to treat or prevent any suitable hematological malignancy in a subject. In certain embodiments, the hematological malignancy can be myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenström's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schöner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenström's macroglobulinaemia, Alpha heavy chain disease, Gamma heavy chain disease, Franklin's disease, Immunoproliferative small intestinal disease, Mediterranean disease, Malignant immunoproliferative disease, unspecified, or Immunoproliferative disease NOS.

In certain embodiments, the methods disclosed herein and throughout can be used to treat or prevent an immune deficiency disease or disorder in a subject. In certain embodiments, the methods disclosed herein and throughout can be used to treat or prevent any suitable an immune deficiency disease or disorder in a subject. In certain embodiments, the immune deficiency disease or disorder can be Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs), or Lymphopenia.

In certain embodiments, the methods disclosed herein and throughout can be used to treat or prevent an autoimmune disease or disorder. In certain embodiments, the autoimmune disease or disorder comprises inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, transplantation rejection, or the like. However, autoimmune diseases is a very active area of research, and further diseases may be identified as the disclosure provided herein and throughout can be obtained by the treatment. Definition of autoimmune disease in which the immune system attacks its own human disease proteins, cells and tissues. A comprehensive review of autoimmune diseases and the list can be found in The Autoimmune Diseases (Rose and Mackay, 2014, Academic Press). The present method can further comprise administering an effective amount of a second therapeutic agent for treating or preventing a proliferation disorder in a subject. In certain embodiments the methods disclosed herein and throughout can be used for treating or preventing a proliferation disease or disorder, e.g., a tumor or a cancer, in a subject and further comprise administering an anti-neoplasm substance to the subject.

In certain embodiments pharmaceutical compositions disclosed herein and throughout are configured to treat or prevent an infectious diseases or disorder in a subject. In certain embodiments, infectious diseases or disorder comprise: Acinetobacter infections, Actinomycosis, African sleeping sickness (African trypanosomiasis), AIDS (acquired immunodeficiency syndrome), Amoebiasis, Anaplasmosis, Angiostrongyliasis Anisakiasis, Anthrax, Arcanobacterium haemolyticum infection, Argentine hemorrhagic fever, Ascariasis, Aspergillosis Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus (children and babies), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, SARS (severe acute respiratory syndrome), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, or Zygomycosis.

To practice the methods disclosed herein and throughout, a modified IL-2 polypeptide, and/or a DNA polynucleotide, an RNA polynucleotide, a non-viral vector, or a viral vector encoding a modified IL-2 polypeptide or conjugate thereof, or a pharmaceutical composition comprising any of the same, as described above, may be administered via any suitable route. In certain embodiments, a modified IL-2 polypeptide and/or a DNA polynucleotide, an RNA polynucleotide, a non-viral vector, or a viral vector encoding a modified IL-2 polypeptide or a conjugate thereof, or a pharmaceutical composition comprising any of the same, as disclosed herein and throughout, may be administered orally, parenterally, by inhalation, topically, rectally, nasally, buccally, vaginally, via an implanted reservoir, or other drug administration methods. The term “parenteral” as used herein and throughout includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

In certain embodiments, the disclosure provided herein and throughout is directed to an use of an effective amount of a modified IL-2 polypeptide and/or a DNA polynucleotide, an RNA polynucleotide, a non-viral vector, or a viral vector encoding a modified IL-2 polypeptide or a conjugate thereof, or a pharmaceutical composition comprising any of the same, as described above, for the manufacture of a medicament for treating or preventing a disease or a disorder, e.g., a proliferation disease or disorder, in a subject.

### Methods for Expanding Various Immune Cells

In certain embodiments, the disclosure provided herein and throughout is directed to a method of expanding a CD25+ Treg, CD4⁺ helper cell, CD8⁺ effector naive and memory cell, Natural Killer (NK) cell, or Natural killer T (NKT) cell population, which comprises contacting a cell population with an effective amount of a modified IL-2 polypeptide and/or a DNA polynucleotide, an RNA polynucleotide, a non-viral vector, or a viral vector encoding a modified IL-2 polypeptide or a conjugate thereof, or a pharmaceutical composition comprising any of the same, as described above, for a time sufficient to induce formation of a complex with an IL-2Rβγ, thereby stimulating the expansion of the Treg cell, CD4⁺ helper cell, CD8⁺ effector naive and memory cell, NK cell, and/or NKT cell population.

In certain embodiments, the disclosure provided herein and throughout is directed to a method of expanding a CD4⁺ helper cell, CD8⁺ effector naive and memory cell, Treg cell, Natural Killer (NK) cell, or Natural killer T (NKT) cell population, which comprises contacting a cell population with an effective amount of a modified IL-2 polypeptide and/or encoding a DNA polynucleotide, an RNA polynucleotide, a non-viral vector, or a viral vector encoding a modified IL-2 polypeptide or a conjugate thereof, or a pharmaceutical composition comprising any of the same, as described above, for a time sufficient to induce formation of a complex with an IL-2Rβγ, thereby stimulating the expansion of the a Treg cell, CD4⁺ helper cell, CD8⁺ effector naive and memory cell, NK cell, and/or NKT cell population with reduced cell death by 10% to 100%, e.g., with reduced cell death by 10%, 20%, 30%, 40%, 50%, 60%, 70%, 80%, 90%, 100% or any subrange thereof.

In certain embodiments, the modified IL-2 polypeptide and/or encoding RNA/DNA/viral vector, the modified IL-2 polypeptide conjugate or pharmaceutical composition, as described above, expands CD25⁺ T regulatory (Treg) cells by more than 1%, 5%, 10%, 20%, 30%, 40%, 50%, 60%, 70%, 80%, 90%, or more in the CD3⁺ cell population compared to an expansion of CD25⁺ Treg cells in the CD3⁺ cell population contacted with a comparable IL-2 polypeptide comprising an amino acid sequence set forth in SEQ ID NO:1 or SEQ ID NO:2 without the substitution. In certain embodiments, the modified IL-2 polypeptide and/or a DNA polynucleotide, an RNA polynucleotide, a non-viral vector, or a viral vector encoding a modified IL-2 polypeptide or a conjugate thereof, or a pharmaceutical composition comprising any of the same, as described above, does not expand CD8⁺ T cells in the cell population. In still another embodiment, the ratio of the Treg cells to Teff cells in the cell population after incubation with the modified IL-2 polypeptide and/or encoding RNA/DNA/viral vector, modified IL-2 polypeptide conjugate or pharmaceutical composition, as described above, is about 1:100, 1:50, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 20:1, 50:1, 100:1 or more.

In certain embodiments, the methods disclosed herein and throughout can be conducted in any suitable manner. In certain embodiments, the methods disclosed herein and throughout are conducted in vivo. In certain embodiments, the methods disclosed herein and throughout are conducted in vitro. In certain embodiments, the methods disclosed herein and throughout are conducted ex vivo.

In certain embodiments, the disclosure provided herein and throughout is directed to a use of an effective amount of a modified IL-2 polypeptide and/or a DNA polynucleotide, an RNA polynucleotide, a non-viral vector, or a viral vector encoding a modified IL-2 polypeptide or a conjugate thereof, or a pharmaceutical composition comprising any of the same, as described above, for the manufacture of a medicament for expanding a Treg cell, CD4⁺ helper cell, CD8⁺ effector naive and memory cell, Natural Killer (NK) cell, or Natural killer T (NKT) cell population in a cell population. In certain embodiments, the present use is configured for expanding a Treg cell, CD4⁺ helper cell, CD8⁺ effector naive and memory cell, Natural Killer (NK) cell, or Natural killer T (NKT) cell population in a subject.

### Other Exemplary Embodiments

Embodiment 1. A modified interleukin 2 (IL-2) polypeptide comprising an amino acid sequence having at least 80% identity to SEQ ID NO:1 or SEQ ID NO:2, wherein the modified IL-2 polypeptide comprises a substitution with a natural or unnatural amino acid at a position selected from the group consisting of L18, L19, N29, Y31, V69, N71, Q74, N88, V91, I128 and a combination thereof, wherein said modified IL-2 polypeptide:
a) has enhanced binding to an interleukin 2 receptor α (IL-2Rα) compared to an IL-2 polypeptide without the substitution; and/or
b) has enhanced binding to an interleukin 2 receptor αβγ (IL-2Rαβγ) compared an IL-2 polypeptide without the substitution; and/or
b) has enhanced binding to cells expressing an interleukin 2 receptor αβγ (IL-2Rαβγ) compared to an IL-2 polypeptide without the substitution; and/or
c) has enhanced receptor signaling potency via IL-2Rαβγ compared to an IL-2 polypeptide without the substitution; and/or
e) has an enhanced ratio of IL-2Rαβγ receptor signaling potency to IL-2Rβγ receptor signaling potency compared to the ratio of IL-2Rαβγ receptor signaling potency to IL-2Rβγ receptor signaling potency of an IL-2 polypeptide without the substitution; and/or
f) is configured to be conjugated to a conjugating moiety; and/or
g) is conjugated to a conjugating moiety; and/or
h) combinations of a) through g).

Embodiment 2. The modified IL-2 polypeptide of Embodiment 1, wherein the modified IL-2 polypeptide comprises:
a) a substitution with cysteine, lysine, histidine, arginine, aspartic acid, glutamic acid, serine, threonine, alanine, tryptophan, isoleucine, phenylalanine, or tyrosine at position N29; and/or
b) a substitution with cysteine, lysine, histidine, arginine, aspartic acid, glutamic acid, serine, threonine, alanine, tryptophan, isoleucine, or phenylalanine at position Y31.

Embodiment 3. The modified IL-2 polypeptide of Embodiment 1 or Embodiment 2, wherein the modified IL-2 polypeptide comprises the substitution N29C.

Embodiment 4. The modified IL-2 polypeptide of any of Embodiments 1-3, wherein the modified IL-2 polypeptide comprises the substitution Y31C.

Embodiment 5. The modified IL-2 polypeptide of Embodiments 1-4, wherein the modified IL-2 polypeptide comprises a substitution with lysine, cysteine, histidine, arginine, aspartic acid, glutamic acid, serine, threonine, alanine, methionine, tryptophan, isoleucine, phenylalanine, proline, or tyrosine at one or more positions selected from the group consisting of L18, L19, V69, Q74, N88, V91, and I128.

Embodiment 6. The modified IL-2 polypeptide of any of Embodiments 1-5, wherein the modified IL-2 polypeptide comprises a substitution selected from the group consisting of Y31C.

Embodiment 7. The modified IL-2 polypeptide of any of Embodiments 1-6, wherein the modified IL-2 polypeptide is conjugated to a conjugating moiety selected from the group consisting of a water-soluble polymer, a lipid, a peptide, a protein, a polypeptide, and combinations thereof.

Embodiment 8. The modified IL-2 polypeptide of any of Embodiments 1-7, wherein the modified IL-2 polypeptide is conjugated to a polyethylene glycol.

Embodiment 9. The modified IL-2 polypeptide of any of Embodiments 1-8, wherein the modified IL-2 polypeptide comprises a mutation selected from the group consisting of N29C, N30C, Y31C, E100C, N119C, T123C, S127C, or T131C, wherein the polypeptide is pegylated at the N29C, N30C, Y31C, E100C, N119C, T123C, S127C, or T131C site.

Embodiment 10. The modified IL-2 polypeptide of any of Embodiments 1-9, wherein the modified IL-2 polypeptide comprises a N29C or Y31C mutation.

Embodiment 11. The modified IL-2 polypeptide of any of Embodiments 1-10, wherein the modified IL-2 polypeptide comprises:
a) a substitution with lysine, cysteine, histidine, arginine, aspartic acid, glutamic acid, serine, threonine, alanine, tryptophan, isoleucine, phenylalanine, or tyrosine at a position selected from the group consisting of N29, N30, Y31 and combinations thereof; or
b) a substitution with lysine, cysteine, histidine, arginine, aspartic acid, glutamic acid, serine, threonine, alanine, tryptophan, isoleucine, phenylalanine, or tyrosine at a position selected from the group consisting of N30, Y31, and combinations thereof.

Embodiment 12. The modified IL-2 polypeptide of any of Embodiments 1-11, wherein the modified IL-2 polypeptide comprises:
a) a substitution with a natural amino acid or an unnatural amino acid at one or more positions selected from the group consisting of N29, N30, Y31, and is:
   (i) unconjugated;
   (ii) conjugated to; or
   (iii) configured to be conjugated to;
   one or more water-soluble polymers, lipids, proteins, or peptides at one or more positions selected from the group consisting of N29, N30, Y31, E100, N119, T123, S127, T131; and/or
b) a substitution with a natural amino acid or an unnatural amino acid at a position selected from the group consisting of N29, N30, Y31, and is:
   (i) unconjugated;
   (ii) conjugated to; or
   (iii) configured to be conjugated to;
   one or more water-soluble polymers, lipids, proteins, or peptides at one or more positions selected from the group consisting of N29, N30, Y31; and/or
c) a substitution with a natural amino acid or an unnatural amino acid at a position selected from the group consisting of N29, N30, Y31 and a combination thereof, and is:
   (i) unconjugated;
   (ii) conjugated to; or
   (iii) configured to be conjugated to;
   one or more water-soluble polymers, lipids, proteins, or peptides at the N terminal and/or C terminal of the modified IL-2 polypeptide.

Embodiment 13. The modified IL-2 polypeptide of any of Embodiments 1-12 wherein the modified IL-2 polypeptide comprises:
a) a substitution with cysteine at one or more positions selected from the group consisting of N29, N30, Y31; and/or
b) a substitution with cysteine at one or more positions selected from the group consisting of N30, Y31; and/or
c) comprises a substitution with cysteine at a position of Y31; and/or
f) comprises a substitution with cysteine at a position of N30.

Embodiment 14. The modified IL-2 polypeptide of any of Embodiments 1-13, wherein the modified IL-2 polypeptide comprises one or more substitutions with a natural amino acid or an unnatural amino acid at a position within IL-2Rα interaction region, and/or IL-2Rβ interaction region and/or IL-2Rγ interaction region.

Embodiment 15. The modified IL-2 polypeptide of any of Embodiments 1-14, wherein the modified IL-2 polypeptide comprises one or more substitutions with a natural amino acid or an unnatural amino acid at a position within IL-2Rβ interaction region and/or IL-2Rγ interaction region.

Embodiment 16. The modified IL-2 polypeptide of any of Embodiments 1-15, wherein the modified IL-2 polypeptide comprises one or more substitutions with a natural amino acid or an unnatural amino acid at a position selected from the group consisting of L18, L19, V69, Q74, N88, V91, I128, and a combination thereof.

Embodiment 17. The modified IL-2 polypeptide of any of Embodiments 1-16, wherein the modified IL-2 polypeptide comprises one or more substitutions with lysine, cysteine, histidine, arginine, aspartic acid, glutamic acid, serine, threonine, alanine, methionine, tryptophan, isoleucine, phenylalanine, proline, or tyrosine at a position selected from the group consisting of L18, L19, V69, Q74, N88, V91, I128, and a combination thereof.

Embodiment 18. The modified IL-2 polypeptide of any of Embodiments 1-17, wherein the modified IL-2 polypeptide comprises:
a) a substitution with methionine at a position L18; and/or
b) a substitution with serine at a position of L19; and/or
c) a substitution with cysteine at position of Y31, and/or
d) comprises a substitution with alanine at a position of V69; and/or
e) comprises a substitution with proline at a position of Q74; and/or
f) comprises a substitution with arginine, aspartic acid, glutamic acid, lysine at a position of N88; and/or
g) comprises a substitution with arginine at a position of N88; and/or
h) comprises a substitution with aspartic acid at a position of N88;
i) comprises a substitution with glutamic acid at a position of N88;
j) comprises a substitution with lysine at a position of N88;
k) comprises a substitution with lysine at a position of V91;
l) comprises a substitution with threonine at a position of I128; and/or
m) combinations of a) through 1).

Embodiment 19. The modified IL-2 polypeptide of any of Embodiments 1-18, wherein the modified IL-2 polypeptide comprises:
a) a substitution with a natural amino acid at a position within IL-2Rα interaction region and a substitution with a natural amino acid at a position within IL-2Rβ interaction region; and/or
b) a substitution with a natural amino acid at a position within IL-2Rα interaction region and a substitution with a natural amino acid at a position within IL-2Rγ interaction region; and/or
c) a substitution with a natural amino acid at a position within IL-2Rα interaction region, a substitution with a natural amino acid at a position within IL-2Rβ interaction region and a substitution with a natural amino acid at a position within IL-2Rγ interaction region.

Embodiment 20. The modified IL-2 polypeptide of any of Embodiments 1-19, wherein the modified IL-2 polypeptide has increased binding to an IL-2Rα and/or IL-2Rαβγ compared to an IL-2 polypeptide comprising an amino acid sequence set forth in SEQ ID NO:1 or SEQ ID NO:2 without the substitution.

Embodiment 21. The modified IL-2 polypeptide of any of Embodiments 1-20, wherein the binding affinity of the modified IL-2 polypeptide to an IL-2Rα and/or IL-2Rαβγ is increased from about 10% to about 100%, or is increased from about 1-fold to about 100,000-fold or more.

Embodiment 22. The modified IL-2 polypeptide of any of Embodiments 1-21, wherein the modified IL-2 polypeptide has increased binding to IL-2Rα expressing cells and/or IL-2Rαβγ expressing cells compared to an IL-2 polypeptide comprising an amino acid sequence set forth in SEQ ID NO:1 or SEQ ID NO:2 without the substitution.

Embodiment 23. The modified IL-2 polypeptide of any of Embodiments 1-22, wherein the modified IL-2 polypeptide has increased binding to IL-2Rα expressing cells and/or IL-2Rαβγ expressing cells that is increased from about 10% to about 100%, or is increased from about 1-fold to about 100,000-fold or more.

Embodiment 24. The modified IL-2 polypeptide of any of Embodiments 1-23, wherein the modified IL-2 polypeptide has reduced internalization by IL-2Rα expressing cells and/or IL-2Rαβγ expressing cells compared to an IL-2 polypeptide comprising an amino acid sequence set forth in SEQ ID NO:1 or SEQ ID NO:2 without the substitution.

Embodiment 25. The modified IL-2 polypeptide of any of Embodiments 1-24, wherein the modified IL-2 polypeptide has internalization by IL-2Rα expressing cells and/or IL-2Rαβγ expressing cells that is from about 10% to about 100%, or is increased from about 1-fold to about 100,000-fold or more.

Embodiment 26. The modified IL-2 polypeptide of any of Embodiments 1-25, wherein the modified IL-2 polypeptide has no detectable internalization by IL-2Rα expressing cells and/or IL-2Rαβγ expressing cells.

Embodiment 27. The modified IL-2 polypeptide of any of Embodiments 1-26, wherein the modified IL-2 polypeptide has increased receptor signaling potency to IL-2Rαβγ compared to an IL-2 polypeptide comprising an amino acid sequence set forth in SEQ ID NO:1 or SEQ ID NO:2 without the substitution.

Embodiment 28. The modified IL-2 polypeptide of any of Embodiments 1-27, wherein the modified IL-2 polypeptide has increased binding to an IL-2Rα compared to an IL-2 polypeptide comprising an amino acid sequence set forth in SEQ ID NO:1 or SEQ ID NO:2 without the substitution and has increased receptor signaling potency to IL-2Rαβγ compared to an IL-2 polypeptide comprising an amino acid sequence set forth in SEQ ID NO:1 or SEQ ID NO:2 without the substitution.

Embodiment 29. The modified IL-2 polypeptide of any of Embodiments 1-28, wherein the modified IL-2 polypeptide has increased binding to an IL-2Rα and/or IL-2Rαβγ, and increased binding on IL-2Rα expressing cells and/or IL-2Rαβγ expressing cells compared an IL-2 polypeptide comprising an amino acid sequence set forth in SEQ ID NO:1 or SEQ ID NO:2 without the substitution, and has reduced internalization by IL-2Rα expressing cells and/or IL-2Rαβγ expressing cells compared to an IL-2 polypeptide comprising an amino acid sequence set forth in SEQ ID NO:1 or SEQ ID NO:2 without the substitution.

Embodiment 30. The modified IL-2 polypeptide of any of Embodiments 1-29, wherein the modified IL-2 polypeptide has: (i) increased binding to an IL-2Rα and/or IL-2Rαβγ; (ii) increased binding on IL-2Rα expressing cells and/or IL-2Rαβγ expressing cells compared to an IL-2 polypeptide comprising an amino acid sequence set forth in SEQ ID NO:1 or SEQ ID NO:2 without the substitution; (iii) no detectable internalization by IL-2Ra expressing cells and/or IL-2Rαβγ expressing cells compared to an IL-2 polypeptide comprising an amino acid sequence set forth in SEQ ID NO:1 or SEQ ID NO:2 without the substitution; (iv) and increased receptor signaling potency to IL-2Rαβγ compared to an IL-2 polypeptide comprising an amino acid sequence set forth in SEQ ID NO:1 or SEQ ID NO:2 without the substitution.

Embodiment 31. The modified IL-2 polypeptide of any of Embodiments 1-30, wherein the modified IL-2 polypeptide has:
reduced binding level to an interleukin 2 receptor β(IL-2Rβ) or an interleukin 2 receptor γ (IL-2Rγ) compared to an IL-2 polypeptide comprising an amino acid sequence set forth in SEQ ID NO:1 or SEQ ID NO:2 without the substitution;
and/or reduced receptor signaling potency to IL-2Rβγ compared to an IL-2 polypeptide comprising an amino acid sequence set forth in SEQ ID NO:1 or SEQ ID NO:2 without the substitution.

Embodiment 32. The modified IL-2 polypeptide of any of Embodiments 1-31, wherein the modified IL-2 polypeptide has lower receptor signaling potency to IL-2Rβγ compared to an IL-2 polypeptide comprising an amino acid sequence set forth in SEQ ID NO:1 or SEQ ID NO:2 without the substitution.

Embodiment 33. The modified IL-2 polypeptide of any of Embodiments 1-32, wherein the modified IL-2 polypeptide has: (i) lower binding level to an IL-2Rβ or an IL-2Rγ compared to an IL-2 polypeptide comprising an amino acid sequence set forth in SEQ ID NO:1 or SEQ ID NO:2 without the substitution; and (ii) lower receptor signaling potency to IL-2Rβγ compared to an IL-2 polypeptide comprising an amino acid sequence set forth in SEQ ID NO:1 or SEQ ID NO:2 without the substitution.

Embodiment 34. The modified IL-2 polypeptide of any of Embodiments 1-33, wherein the modified IL-2 polypeptide has increased ratio between its signaling potency to IL-2Rαβγ and the signaling potency to IL-2Rβγ compared to an IL-2 polypeptide comprising an amino acid sequence set forth in SEQ ID NO:1 or SEQ ID NO:2 without the substitution.

Embodiment 35. The modified IL-2 polypeptide of any of Embodiments 1-34, wherein the modified IL-2 polypeptide has increased ratio between its signaling potency to IL-2Rαβγ and the signaling potency to IL-2Rβγ is more than 1-fold, more than 10-fold, more than 100-fold, more than 1,000-fold, more than 10,000-fold, more than 100,000-fold.

Embodiment 36. The modified IL-2 polypeptide of any of Embodiments 1-35, wherein the modified IL-2 polypeptide comprises an N terminal deletion, wherein said deletion comprises a deletion of one or more of amino acid residues 1 through 30, inclusive, that are present in the corresponding IL-2 modified polypeptide that does not comprise said N-terminal deletion.

Embodiment 37. The modified IL-2 polypeptide of any of Embodiments 1-36, wherein the modified IL-2 polypeptide comprises a C terminal deletion, wherein said deletion comprises a deletion of one or more of amino acid residues 114 through 134, inclusive, that are present in the corresponding IL-2 modified polypeptide that does not comprise said C-terminal deletion.

Embodiment 38. The modified IL-2 polypeptide of any of Embodiments 1-37, wherein the modified IL-2 polypeptide comprises a N terminal deletion and a C terminal deletion.

Embodiment 39. The modified IL-2 polypeptide of any of Embodiments 1-38, wherein the modified IL-2 polypeptide is a part of a fusion polypeptide comprising an additional amino acid sequence.

Embodiment 40. The modified IL-2 polypeptide of any of Embodiments 1-39, wherein the modified IL-2 polypeptide comprises a recombinant fusion protein comprising the modified IL-2 polypeptide and an additional amino acid sequence.

Embodiment 41. The modified IL-2 polypeptide of any of Embodiments 1-40, wherein the N terminus or the C terminus of the modified IL-2 polypeptide is fused to an additional amino acid sequence.

Embodiment 42. The modified IL-2 polypeptide of any of Embodiments 1-41, wherein the N terminus or the C terminus of the modified IL-2 polypeptide is fused to an additional amino acid sequence, wherein said additional amino acid sequence comprises an antibody sequence or a portion or a fragment thereof.

Embodiment 43. The modified IL-2 polypeptide of any of Embodiments 1-42, wherein the N terminus or the C terminus of the modified IL-2 polypeptide is fused to an additional amino acid sequence, wherein said additional amino acid sequence comprises an Fc portion of an antibody or a portion or a fragment thereof.

Embodiment 44. The modified IL-2 polypeptide of any of Embodiments 1-43, wherein the modified IL-2 polypeptide is isolated.

Embodiment 45. The modified IL-2 polypeptide of any of Embodiments 1-44, wherein the modified IL-2 polypeptide is expressed from a vector comprising a polynucleotide sequence that encodes the modified IL-2 polypeptide.

Embodiment 46. The modified IL-2 polypeptide of any of Embodiments 1-45, wherein the modified IL-2 polypeptide is expressed from a vector comprising a polynucleotide sequence that encodes the modified IL-2 polypeptide, wherein said vector is an RNA vector, a DNA, a viral vector, or a non-viral vector.

Embodiment 47. A modified IL-2 polypeptide, which comprises a modified IL-2 polypeptide of any of Embodiments 1-46 that is conjugated to a water-soluble polymer, a lipid, a polypeptide, a protein, or a peptide.

Embodiment 48. The modified IL-2 polypeptide of any of Embodiments 1-47, wherein the modified IL-2 polypeptide is conjugated to one or more water-soluble polymers, lipids, proteins, or peptides through one or more covalent bonds.

Embodiment 49. The modified IL-2 polypeptide of any of Embodiments 1-48, wherein the modified IL-2 polypeptide is conjugated to one or more water-soluble polymers, lipids, proteins, or peptides through one or more non-covalent bonds.

Embodiment 50. The modified IL-2 polypeptide of any of Embodiments 1-49, wherein the modified IL-2 polypeptide is conjugated to a water-soluble polymer, a lipid, a protein, or a peptide via a substituted natural amino acid or unnatural amino acid at a position selected from the group consisting of L18, L19, N30, Y31, V69, Q74, N88, V91, I128, E100, N119, T123, S127, T131, and combinations thereof.

Embodiment 51. The modified IL-2 polypeptide any of Embodiments 1-50, wherein the modified IL-2 polypeptide is conjugated to a water-soluble polymer, a lipid, a protein, or a peptide via a substituted natural amino acid at a position selected from the group consisting of L18, L19, N30, Y31, V69, Q74, N88, V91, I128, E100, N119, T123, S127, T131, and combinations thereof.

Embodiment 52. The modified IL-2 polypeptide any of Embodiments 1-51, wherein the modified IL-2 polypeptide is conjugated to a water-soluble polymer, a lipid, a protein, or a peptide via a substituted lysine, cysteine, histidine, arginine, aspartic acid, glutamic acid, serine, threonine, alanine, tryptophan, isoleucine, phenylalanine, or tyrosine at a position selected from the group consisting of L18, L19, N30, Y31, V69, Q74, N88, V91, I128, E100, N119, T123, S127, T131, and combinations thereof.

Embodiment 53. The modified IL-2 polypeptide of any of Embodiments 1-52, wherein the modified IL-2 polypeptide is conjugated to a water-soluble polymer, a lipid, a protein, or a peptide via a substituted cysteine at a position selected from the group consisting of L18, L19, N30, Y31, V69, Q74, N88, V91, I128, E100, N119, T123, S127, T131, and combinations thereof.

Embodiment 54. The modified IL-2 polypeptide of any of Embodiments 1-53, wherein the modified IL-2 polypeptide is conjugated to a water-soluble polymer, a lipid, a protein, or a peptide via a substituted natural amino acid or unnatural amino acid at a position selected from the group consisting of L18, L19, N30, Y31, V69, Q74, N88, V91, I128, E100, N119, T123, S127, T131, and combinations thereof.

Embodiment 55. The modified IL-2 polypeptide any of Embodiments 1-54, wherein the modified IL-2 polypeptide is conjugated to a water-soluble polymer, a lipid, a protein, or a peptide via a substituted natural amino acid at a position selected from the group consisting of L18, L19, N30, Y31, V69, Q74, N88, V91, I128, E100, N119, T123, S127, T131, and combinations thereof.

Embodiment 56. The modified IL-2 polypeptide of Embodiments 1-55, wherein the modified IL-2 polypeptide is conjugated to a water-soluble polymer, a lipid, a protein, or a peptide via a substituted lysine, cysteine, histidine, arginine, aspartic acid, glutamic acid, serine, threonine, alanine, tryptophan, isoleucine, phenylalanine, or tyro sine at a position selected from the group consisting of L18, L19, N30, Y31, V69, Q74, N88, V91, I128, E100, N119, T123, S127, T131, and combinations thereof.

Embodiment 57. The modified IL-2 polypeptide of Embodiments 1-56, wherein the modified IL-2 polypeptide is conjugated to a water-soluble polymer, a lipid, a protein, or a peptide via a substituted cysteine at a position selected from the group consisting of L18, L19, N30, Y31, V69, Q74, N88, V91, I128, E100, N119, T123, S127, T131, and combinations thereof.

Embodiment 58. The modified IL-2 polypeptide of any of Embodiments 1-57, wherein the modified IL-2 polypeptide is conjugated to a water-soluble polymer, a lipid, a protein, or a peptide via a single amino acid residue of the modified IL-2 polypeptide.

Embodiment 59. The modified IL-2 polypeptide of any of Embodiments 1-58, wherein the modified IL-2 polypeptide is conjugated to a water-soluble polymer, a lipid, a protein, or a peptide via:
i) the alpha amino group of the N-terminal amino acid residue of the modified IL-2 polypeptide;
ii) the epsilon amino group of a lysine amino acid residue of the modified IL-2 polypeptide; or
iii) an N-glycosylation site or O-glycosylation site of the modified IL-2 polypeptide.

Embodiment 60. The modified IL-2 polypeptide of any of Embodiments 1-59, wherein the modified IL-2 polypeptide is covalently conjugated to a water-soluble polymer, a lipid, a protein, or a peptide through a linker.

Embodiment 61. The modified IL-2 polypeptide of any of Embodiments 1-60, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence.

Embodiment 62. The modified IL-2 polypeptide of any of Embodiments 1-61, wherein the modified IL-2 polypeptide is conjugated to a water-soluble polymer, a lipid, a protein, or a peptide via a single amino acid residue located within the modified IL-2 polypeptide.

Embodiment 63. The modified IL-2 polypeptide of any of Embodiments 1-62, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the single amino acid residue is located within the additional amino acid sequence.

Embodiment 64. The modified IL-2 polypeptide of any of Embodiments 1-63, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the additional amino acid sequence comprises an antibody sequence or a portion or a fragment thereof.

Embodiment 65. The modified IL-2 polypeptide of any of Embodiments 1-64, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the additional amino acid sequence comprises a Fc portion of an antibody.

Embodiment 66. The modified IL-2 polypeptide of any of Embodiments 1-65, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the single amino acid residue is:
i) the alpha amino group of the N-terminal amino acid residue of the fusion polypeptide;
ii) the epsilon amino group of a lysine amino acid residue of the fusion polypeptide; or
iii) an N-glycosylation site or O-glycosylation site of the fusion polypeptide.

Embodiment 67. The modified IL-2 polypeptide of any of Embodiments 1-65, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the fusion polypeptide is covalently conjugated to the water-soluble polymer, a lipid, a protein, or a peptide through a linker.

Embodiment 68. The modified IL-2 polypeptide of any of Embodiments 1-67, wherein the modified IL-2 polypeptide is conjugated to a water-soluble polymer.

Embodiment 69. The modified IL-2 polypeptide of any of Embodiments 1-68, wherein the modified IL-2 polypeptide is conjugated to a water-soluble polymer comprising polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or combinations thereof.

Embodiment 70. The modified IL-2 polypeptide of any of Embodiments 1-69, wherein the modified IL-2 polypeptide is conjugated to a water-soluble polymer comprising a PEG molecule.

Embodiment 71. The modified IL-2 polypeptide of any of Embodiments 1-70, wherein the modified IL-2 polypeptide is conjugated to a water-soluble polymer comprising a linear PEG molecule.

Embodiment 72. The modified IL-2 polypeptide of any of Embodiments 1-71, wherein the modified IL-2 polypeptide is conjugated to a water-soluble polymer comprising a branched PEG molecule.

Embodiment 73. The modified IL-2 polypeptide of any of Embodiments 1-72, wherein the modified IL-2 polypeptide is conjugated to a water-soluble polymer comprising a branched PEG molecule comprising about three to about ten PEG chains emanating from a central core group.

Embodiment 74. The modified IL-2 polypeptide of any of Embodiments 1-73, wherein the modified IL-2 polypeptide is conjugated to a water-soluble polymer comprising a branched PEG molecule, wherein the branched PEG molecule is a star PEG comprising from about 10 to about 100 PEG chains emanating from a central core group.

Embodiment 75. The modified IL-2 polypeptide of any of Embodiments 1-74, wherein the modified IL-2 polypeptide is conjugated to a water-soluble polymer comprising a branched PEG molecule, wherein the branched PEG molecule is a comb PEG comprising multiple PEG chains grafted onto a polymer backbone.

Embodiment 76. The modified IL-2 polypeptide of any of Embodiments 1-75, wherein the modified IL-2 polypeptide is conjugated to a water-soluble polymer comprising a PEG molecule, wherein the PEG molecule has a range of molecular weight from about 300 g/mol to about 10,000,000 g/mol.

Embodiment 77. The modified IL-2 polypeptide of any of Embodiments 1-76, wherein the modified IL-2 polypeptide is conjugated to a water-soluble polymer comprising a PEG molecule, wherein the PEG molecule has an average molecular weight from about 5,000 Daltons to about 1,000,000 Daltons.

Embodiment 78. The modified IL-2 polypeptide of any of Embodiments 1-77, wherein the modified IL-2 polypeptide is conjugated to a water-soluble polymer comprising a PEG molecule, wherein the PEG molecule has an average molecular weight of from about 20,000 Daltons to about 30,000 Daltons.

Embodiment 79. The modified IL-2 polypeptide of any of Embodiments 1-78, wherein the modified IL-2 polypeptide is conjugated to a water-soluble polymer comprising a PEG molecule, wherein the PEG molecule is a monodisperse, uniform, or discrete PEG molecule.

Embodiment 80. The modified IL-2 polypeptide of any of Embodiments 1-79, wherein the modified IL-2 polypeptide is conjugated to a water-soluble polymer, wherein the water-soluble polymer comprises a polysaccharide.

Embodiment 81. The modified IL-2 polypeptide of any of Embodiments 1-80, wherein the modified IL-2 polypeptide is conjugated to a lipid.

Embodiment 82. The modified IL-2 polypeptide of any of Embodiments 1-81, wherein the modified IL-2 polypeptide is conjugated to a lipid, wherein the lipid comprises a fatty acid.

Embodiment 83. The modified IL-2 polypeptide of any of Embodiments 1-82, wherein the modified IL-2 polypeptide is conjugated to a protein.

Embodiment 84. The modified IL-2 polypeptide of any of Embodiments 1-83, wherein the modified IL-2 polypeptide is conjugated to a protein, wherein the protein comprises an antibody or a binding fragment thereof.

Embodiment 85. The modified IL-2 polypeptide of any of Embodiments 1-84, wherein the modified IL-2 polypeptide is conjugated to an Fc portion of an antibody or a fragment thereof.

Embodiment 86. The modified IL-2 polypeptide of any of Embodiments 1-85, wherein the modified IL-2 polypeptide is conjugated to a water-soluble polymer, a lipid, a protein, or a peptide that is indirectly bound to the substituted natural amino acid or unnatural amino acid of the modified IL-2 polypeptide through a linker.

Embodiment 87. The modified IL-2 polypeptide of any of Embodiments 1-86, wherein the modified IL-2 polypeptide is conjugated to a water-soluble polymer, a lipid, a protein, or a peptide that is directly bound to the substituted natural amino acid or unnatural amino acid of the modified IL-2 polypeptide.

Embodiment 88. The modified IL-2 polypeptide of any of Embodiments 1-87, wherein the modified IL-2 polypeptide, wherein the modified IL-2 polypeptide has a half-life in vivo from about 5 minutes to about 10 days.

Embodiment 89. The modified IL-2 polypeptide of any of Embodiments 1-88, wherein the modified IL-2 polypeptide is selected from the group consisting of ACT5200, ACT5201, ACT5210, ACT5211, ACT5212, ACT522S0, ACT522S1, ACT5230, ACT5231, ACT5260, ACT5261, ACT5270, ACT5271, ACT5280, ACT5281, ACT5290, and ACT5291.

Embodiment 90. A pharmaceutical composition comprising an effective amount of a modified IL-2 polypeptide of any of Embodiments 1-89 and a pharmaceutically acceptable carrier or excipient.

Embodiment 91. The pharmaceutical composition of Embodiment 90, wherein the pharmaceutical composition further comprises another active ingredient.

Embodiment 92. The pharmaceutical composition of Embodiment 90 or Embodiment 91, further comprising one or more additional ingredients, wherein the one or more active ingredients comprises:
(i) an anti-inflammatory substance or an anti-autoimmune substance;
(ii) an anti-neoplasm substance;
(iii) an anti-infectious disease substance; and/or
(iv) an immune deficiency disorder.

Embodiment 93. The modified IL-1 polypeptide of any of Embodiments 1-89 or the pharmaceutical composition of any of Embodiments 90-92 for use in treating or preventing a disease or disorder in a subject having, or suspected of having, the disease or disorder.

Embodiment 94. The modified IL-1 polypeptide of any of Embodiments 1-89 or the pharmaceutical composition of any of Embodiments 90-92 for use in treating or preventing a disease or disorder in a subject having, or suspected of having, the disease or disorder, wherein the disease or disorder comprises an inflammatory disease or disorder or an autoimmune disease or disorder.

Embodiment 95. The modified IL-1 polypeptide of any of Embodiments 1-89 or the pharmaceutical composition of any of Embodiments 90-92 for use in treating or preventing a disease or disorder in a subject having, or suspected of having, the disease or disorder, wherein the disease or disorder comprises a proliferation disease or disorder.

Embodiment 96. The modified IL-1 polypeptide of any of Embodiments 1-89 or the pharmaceutical composition of any of Embodiments 90-92 for use in treating or preventing a disease or disorder in a subject having, or suspected of having, the disease or disorder, wherein the disease or disorder comprises an infectious disease or disorder.

Embodiment 97. The modified IL-1 polypeptide of any of Embodiments 1-89 or the pharmaceutical composition of any of Embodiments 90-92 for use in treating or preventing a disease or disorder in a subject having, or suspected of having, the disease or disorder, wherein the disease or disorder comprises an immune deficiency disorder.

Embodiment 98. A method for treating or preventing a disease or a disorder in a subject having, or suspected of having, the disease or disorder, comprising administering to said subject an effective amount of a modified IL-2 polypeptide of any of Embodiments 1-89, or a pharmaceutical composition of any of Embodiments 90-92.

Embodiment 99. A method for treating or preventing a disease or a disorder in a subject having, or suspected of having, the disease or disorder, comprising administering to said subject an effective amount of a modified IL-2 polypeptide of any of Embodiments 1-89, or a pharmaceutical composition of any of Embodiments 90-92, wherein the disease or disorder comprises an inflammatory disease or disorder or an autoimmune disease or disorder.

Embodiment 100. A method for treating or preventing a disease or a disorder in a subject having, or suspected of having, the disease or disorder, comprising administering to said subject an effective amount of a modified IL-2 polypeptide of any of Embodiments 1-89, or a pharmaceutical composition of any of Embodiments 90-92, wherein the disease or disorder comprises a proliferation disease or disorder.

Embodiment 101. A method for treating or preventing a disease or a disorder in a subject having, or suspected of having, the disease or disorder, comprising administering to said subject an effective amount of a modified IL-2 polypeptide of any of Embodiments 1-89, or a pharmaceutical composition of any of Embodiments 90-92, wherein the disease or disorder comprises an infectious disease or disorder.

Embodiment 102. A method for treating or preventing a disease or a disorder in a subject having, or suspected of having, the disease or disorder, comprising administering to said subject an effective amount of a modified IL-2 polypeptide of any of Embodiments 1-89, or a pharmaceutical composition of any of Embodiments 90-92, wherein the disease or disorder comprises an immune deficiency disease or disorder.

Embodiment 103. The use according to any of Embodiments 93-97 or the method of any of Embodiments 98-102, wherein the subject is a human.

Embodiment 104. The use according to any of Embodiments 93-97 or the method of any of Embodiments 97-102, wherein the subject is a non-human mammal.

Embodiment 105. The use according to Embodiment 95 or the method of Embodiment 100, wherein the proliferation disorder comprises a tumor.

Embodiment 106. The use according to Embodiment 95 or the method of Embodiment 100, wherein the proliferation disorder comprises a cancer.

Embodiment 107. The use according to Embodiment 95 or the method of Embodiment 100, wherein the proliferation disorder comprises a solid tumor or a cancer.

Embodiment 108. The use according to Embodiment 95 or the method of Embodiment 100, wherein the proliferation disorder comprises a solid tumor or a cancer, wherein the solid tumor or the cancer is selected from the group consisting of: Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor and Liposarcoma.

Embodiment 109. The use according to Embodiment 95 or the method of Embodiment 100, wherein the proliferation disorder comprises a tumor or a cancer, wherein the tumor or cancer is a hematological malignancy.

Embodiment 110. The use according to Embodiment 95 or the method of Embodiment 100, wherein the proliferation disorder comprises a tumor or a cancer, wherein the tumor or cancer is a hematological malignancy selected from the group consisting of: myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenström's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schöner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenström's macroglobulinaemia, Alpha heavy chain disease, Gamma heavy chain disease, Franklin's disease, Immunoproliferative small intestinal disease, Mediterranean disease, Malignant immunoproliferative disease, unspecified, and Immunoproliferative disease NOS.

Embodiment 111. The use according to Embodiment 94 or the method of Embodiment 99, wherein the inflammatory disease or disorder or the autoimmune disease or disorder is selected from the group consisting of: inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, and transplantation rejection.

Embodiment 112. The use according to Embodiment 96 or the method of Embodiment 101, wherein the infectious disease is selected from the group consisting of: Acinetobacter infections, Actinomycosis, African sleeping sickness (African trypanosomiasis), AIDS (acquired immunodeficiency syndrome), Amoebiasis, Anaplasmosis, Angiostrongyliasis Anisakiasis, Anthrax, Arcanobacterium haemolyticum infection, Argentine hemorrhagic fever, Ascariasis, Aspergillosis Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus (children and babies), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, SARS (severe acute respiratory syndrome), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, and Zygomycosis.

Embodiment 113. The use according to Embodiment 97 or the method of Embodiment 102, wherein the immune deficiency disease or disorder is selected from the group consisting of: Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs), and Lymphopenia.

Embodiment 114. Use of an effective amount of a modified IL-2 polypeptide of any of Embodiments 1-89, or an RNA polynucleotide, a DNA polynucleotide, a non-viral vector, or a viral vector encoding a modified IL-2 polypeptide of any of Embodiments 1-89, for the manufacture of a medicament for treating or preventing a disease or a disorder in a subject.

Embodiment 115. The use according to Embodiment 114, wherein the disease or disorder is selected from the group consisting of: an inflammatory disease or disorder; an autoimmune disease or disorder; a proliferative disease or disorder; an infectious disease or disorder; and an immune deficiency disease or disorder.

Embodiment 116. A method of expanding a Treg cell population, which comprises contacting a cell population with an effective amount of a modified IL-2 polypeptide of any of Embodiments 1-89, or an RNA polynucleotide, a DNA polynucleotide, a non-viral vector, or a viral vector encoding a modified IL-2 polypeptide of any of Embodiments 1-89, for a time sufficient to induce formation of a complex with an IL-2Rαβγ, thereby stimulating the expansion of the Treg cell population.

Embodiment 117. A method of expanding a Treg cell population, which comprises contacting a cell population with an effective amount of a modified IL-2 polypeptide of any of Embodiments 1-89, or an RNA polynucleotide, a DNA polynucleotide, a non-viral vector, or a viral vector encoding a modified IL-2 polypeptide of any of Embodiments 1-89, for a time sufficient to induce formation of a complex with an IL-2Rαβγ, thereby stimulating the expansion of the Treg cell population with reduced cell death by 10% to 100%.

Embodiment 118. The method of Embodiment 116 or 117, wherein the effective amount causes expansion of CD25⁺ T regulatory (Treg) cells by at least 1-fold, 10-fold, 100-fold, 1,000-fold, 10⁴-fold, 10⁵-fold, 10⁶-fold, 10⁷-fold, 10⁸-fold, or 10⁹-fold greater that the expansion of CD25⁺ Treg cells caused with an IL-2 polypeptide comprising an amino acid sequence set forth in SEQ ID NO:1 or SEQ ID NO:2 without the substitution.

Embodiment 119. The method of Embodiment 116 or 117, wherein the effective amount causes an increased the percentage of Treg cells in the T cell population after incubation with the effective amount, compared with an IL-2 polypeptide comprising an amino acid sequence set forth in SEQ ID NO:1 or SEQ ID NO:2 without the substitution, and percentage of the Treg cells is about or at least 0.01%, 0.1%, 1%, 5%, 10%, 20%, 30%, 40%, 50%, 60%, 70%, 80%, 90% or more.

Embodiment 120. The method of any of Embodiments 116-119, wherein the method is conducted in vivo.

Embodiment 121. The method of any of Embodiments 116-119, wherein the method is conducted in vitro.

Embodiment 122. The method of any of Embodiments 116-119, wherein the method is conducted ex vivo.

Embodiment 123. Use of an effective amount of a modified IL-2 polypeptide of any of Embodiments 1-89, or an RNA polynucleotide, a DNA polynucleotide, a non-viral vector, or a viral vector encoding a modified IL-2 polypeptide of any of Embodiments 1-89, for the manufacture of a medicament for expanding a Treg cell in a cell population.

Embodiment 124. The use of Embodiment 123, wherein the Treg cells are expanded in a subject.

### EXAMPLES

### Example 1. Design of the PEG Modified IL-2 Muteins

Selection of IL-2Rα enhancement mutation sites in IL-2. From the human IL-2 peptide sequence modified to include the C125S mutation (see, for example, Aldesleukin. DrugBank. (DB00041 (BTD00082, BIOD00082), one or more of the amino acids from the list of “Site 1” (Table 1) are selected and substituted with other amino acids like cysteine, serine or others. The muteins were expected to have enhanced binding to IL-2Rα and/or IL-2Rαβγ compared with the native IL-2 molecule. All constructs were made in the background of wild-type human IL-2 with a cysteine-to-serine substitution at position 125 (i.e., C125S) to remove this unpaired cysteine residue in IL-2 (called here rhIL-2 or rhIL2; SEQ ID NO:1). Exemplary pegylated IL-2 mutein molecules have enhanced affinity for IL-2Rα, robust binding to IL-2Rαβγ, and reduced receptor mediated internalization and extended half-life in humans and other animals.

TABLE 1
IL-2 mutation design

| IL-2Rα interaction | Pegylated | Other |
|---|---|---|
| enhancement sites (Site 1) | sites (Site 2) | sites (Site 3) |
| N29 | N29C | L18 |
| N30 | N30C | L19 |
| Y31 | Y31C | R81 |
| | Q74C | L85 |
| | K76C | S87 |
| | E100C | V91 |
| | N119C | I92 |
| | T123C | |
| | S127C | |
| | T131C | |
| | N-terminal | |
| | C-terminal | |

Selection of PEG attachment sites in IL-2. In addition to mutations resulting in the enhancement of binding to IL-2Rα and/or IL-2Rαβγ, other mutations and/or modifications were introduced from the “Site 2” column shown in Table 1. Exemplary muteins were conjugated with maleimide-activated PEG. Other PEG reagents may be used as well.

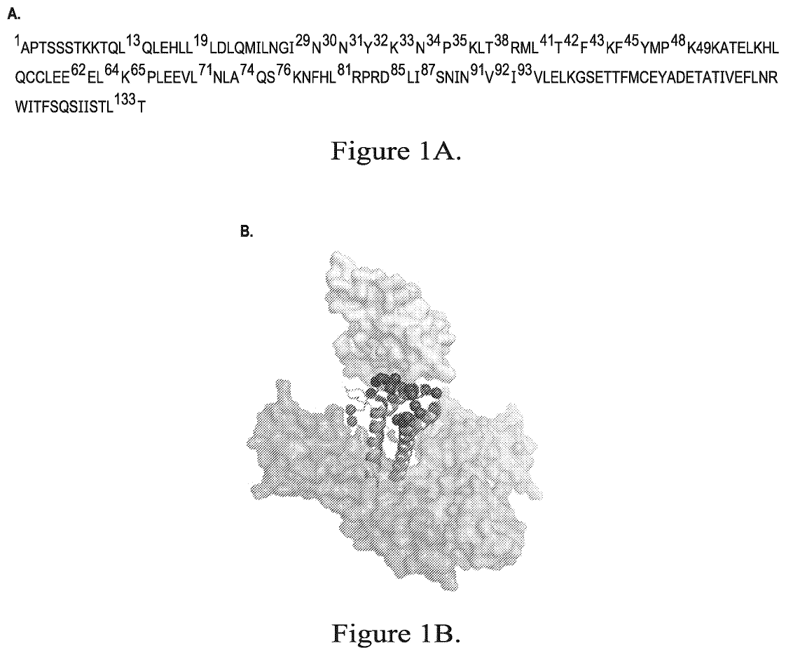

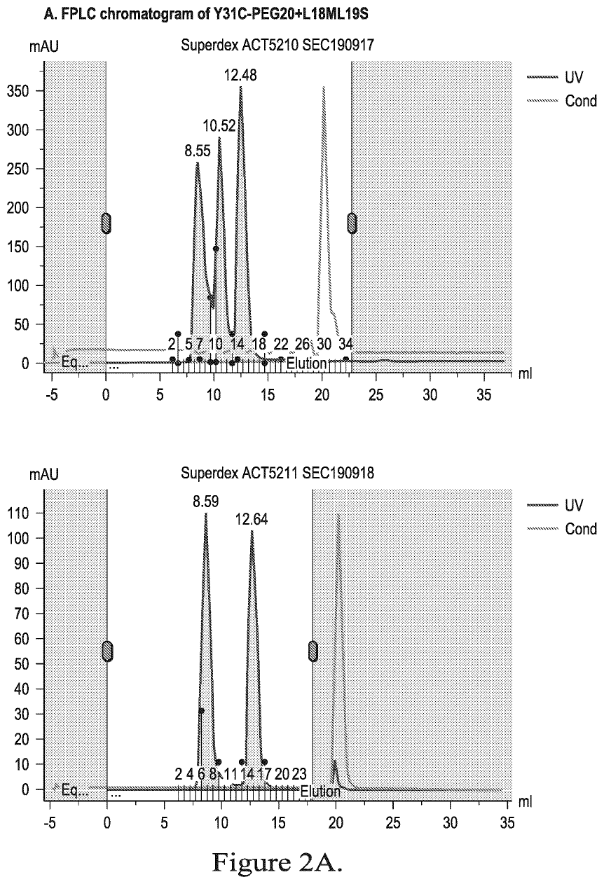

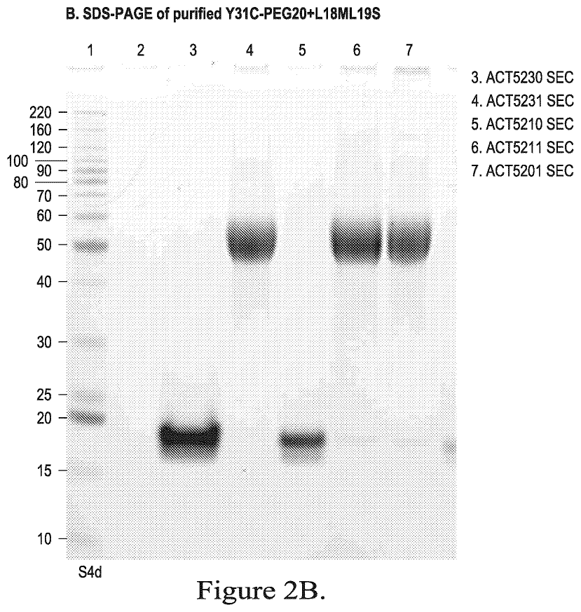

As shown in Table 1, certain mutations belong to both Site 1 and Site 2, and therefore, provide properties associated with both Site 1 and Site 2 to modified IL-2 polypeptides comprising one or more of such mutations. Additional modification of such modified IL-2 polypeptides, such as via PEGylation are expected to have extended half-life compared with the native IL-2 molecule (as well as the rhIL-2C125S polypeptide). Although such PEGylated polypeptides may display some difference in binding affinity for IL-2Rα or IL-2Rαβγ, such differences are expected to be minimal (FIG. 1, Table 1).

Selection of additional mutation sites that reduce IL-2Rα mediated internalization and/or reduced the binding to/activation of IL-2Rβ. In addition to the modifications listed in Site 1 and Site 2 of Table 1, additional modifications were introduced. The modifications carried one or more mutations that substitute an amino acid from the list of “Site 3” (Tablet) with any other amino acids. Mutations at these sites enhanced or further enhanced binding to IL-2Rα, reduced receptor mediated internalization, and/or reduced binding/activation of IL-2Rβγ.

### Example 2. Production and Purification of IL-2 Muteins

cDNAs encoding IL-2 muteins were synthesized and cloned into the pcDNA3.1 (−) vector. Certain exemplary mutein polypeptides (and polynucleotides encoding them) are depicted in Table 2.

TABLE 2
Code names of IL-2 muteins

| Code | Mutation | Code | Mutation | Code | Mutation |
|---|---|---|---|---|---|
| ACT5200 | Y31C | ACT5201 | Y31C (PEG20K) | | |
| ACT5210 | Y31C + L18M + L19S | ACT5211 | Y31C + L18M + L19S (PEG20K) | ACT5212 | Y31C + L18M + L19S |
| | | | | | (PEG30K) |
| ACT522S0 | Y31C + N30S | ACT522S1 | Y31C + N30S (PEG20K) | | |
| ACT5230 | Y31C + V69A + Q74P | ACT5231 | Y31C + V69A + Q74P (PEG20K) | | |
| ACT5260 | Y31C + L18M + L19S + | ACT5261 | Y31C + L18M + L19S + | | |
| | V69A + Q74P | | V69A + Q74P (PEG20K) | | |
| ACT5270 | Y31C + V69A + Q74P + | ACT5271 | Y31C + V69A + Q74P + | | |
| | N30S | | N30S (PEG20K) | | |
| ACT5280 | Y31C + V69A + Q74P + | ACT5281 | Y31C + V69A + Q74P + | | |
| | N88R | | N88R (PEG20K) | | |
| ACT5290 | Y31C + V69A + Q74P + | ACT5291 | Y31C + V69A + Q74P + | | |
| | V91K | | V91K (PEG20K) | | |

Exemplary modified IL-2 polypeptides were expressed as His-Tag fusions by transiently transfecting HEK293F cells with PEI MAX (Polysciences) and culturing the transfected cells for 96 hours. Supernatants were harvested by centrifugation at 4000×g for 20 minutes.

Standard protein purification techniques were then used to isolate the modified IL-2 polypeptides from the supernatant. In brief, the modified IL-2 polypeptides were each captured on a Complete® His-Tag Purification column (Roche) and polished using a Superdex 75 Increase column (GE Healthcare). Purified proteins were eluted from the Superdex 75 Increase column (GE Healthcare) in buffer containing 0.1M MES and 150 mM NaCl, pH 6.0 and were stored at −80° C. for further use.

### Example 3. PEGylation of IL-2 Muteins

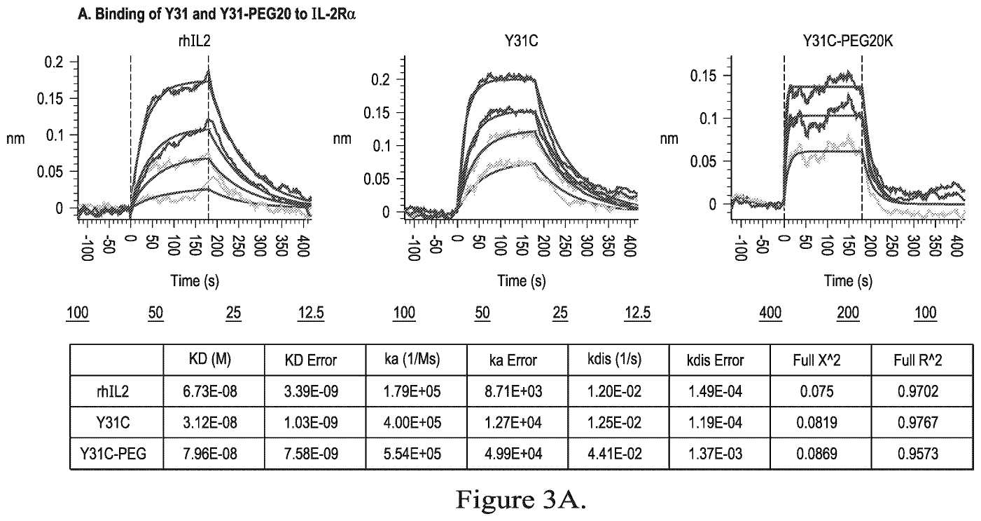

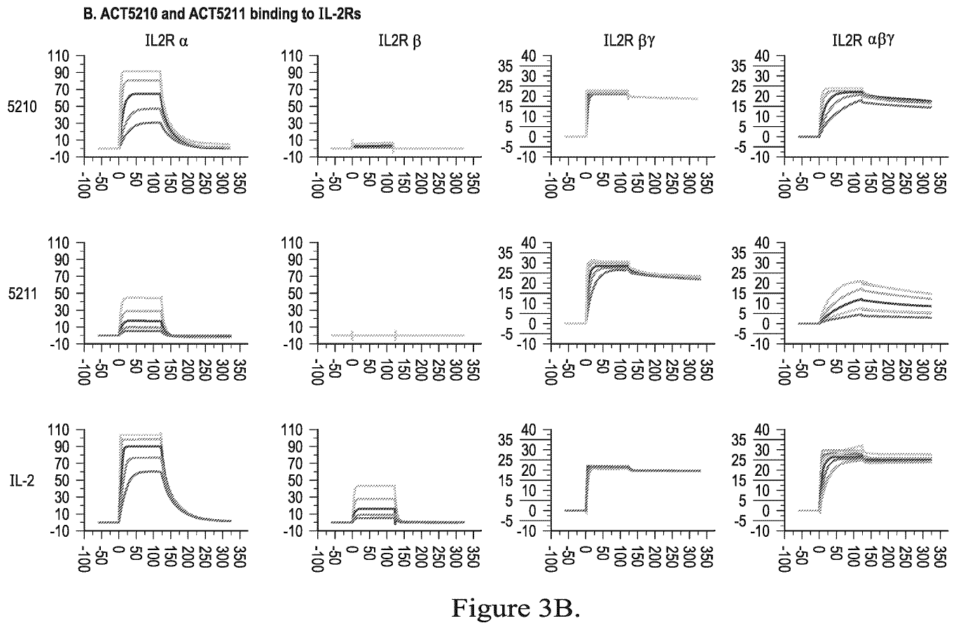

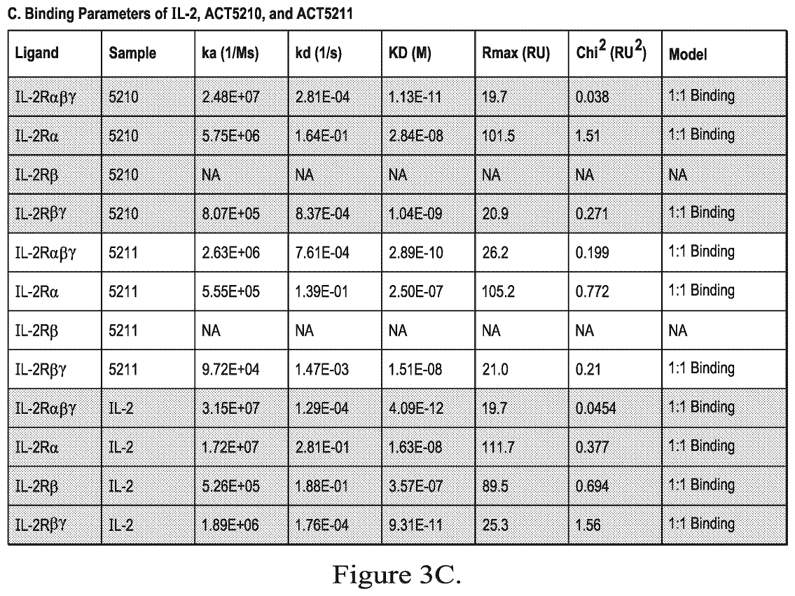

Purified IL-2 muteins (1 mg/ml) were reduced by 5 mM TCEP (Thermo Fisher) at room temperature for 15 min and then reacted with a 50-fold molar excess of maleimide-PEG 20K (Laysan Bio) for 30 minutes at room temperature. The reaction was stopped by adding L-cysteine (Sigma) to a 2-fold molar excess over maleimide-PEG 20k. PEG-conjugates were further purified on the SP Sepharose FF column, followed by the Superdex 75 Increase column (GE Healthcare). Representative chromatogram and SDS-PAGE analysis of the purification process were shown in FIG. 3.

### Example 4. In Vitro Binding of IL-2 Muteins and PEG-Conjugates with IL-2 Receptors

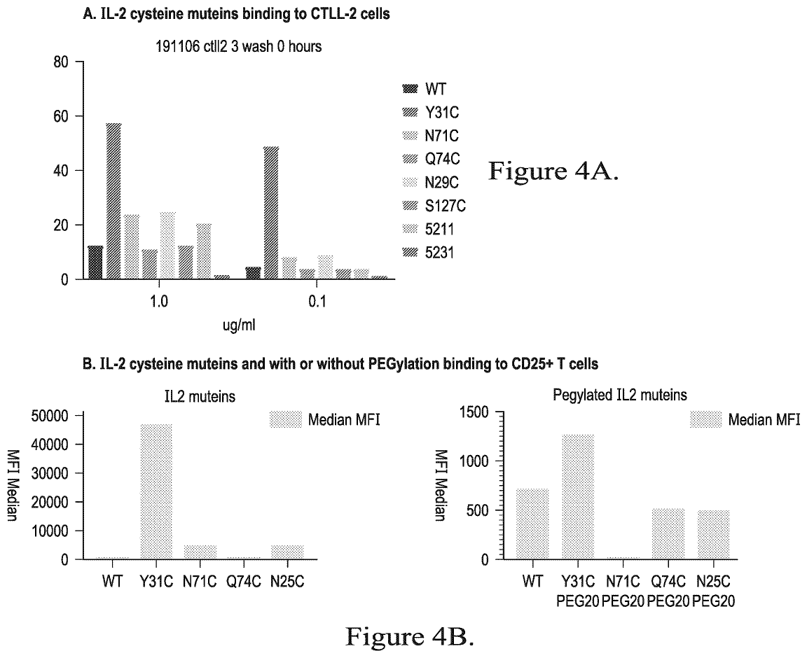

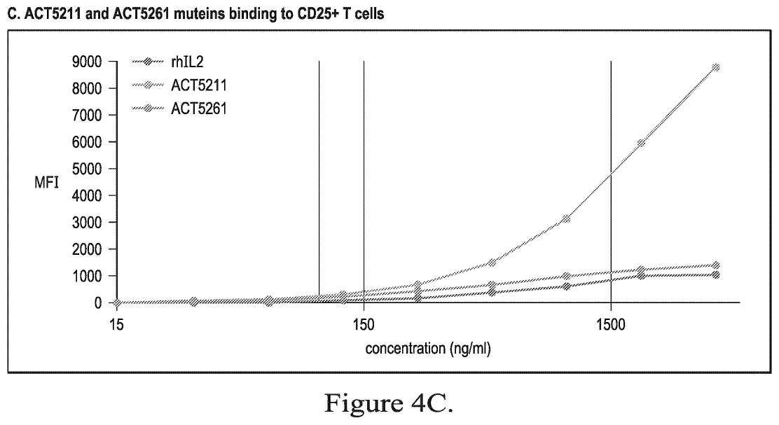

Binding affinities of purified IL-2 muteins and PEG-conjugates with IL-2 receptors were determined by the Octet QKe (ForteBio). IL-2Rα or IL-2Rβ in a human Fc fusion protein format (ACROBiosystems) were captured on anti-human IgG Fc capture (AHC) sensors. After the baseline was established in 1× Kinetics buffer, the sensors were dipped into wells containing serial diluted rhIL-2, IL-2 muteins, or PEG-conjugated IL-2 muteins to measure association constants. Dissociation was detected following transfer of sensors into wells containing buffer alone. Data were collected and analyzed by the Octet User Software. For analysis of the kinetics constants, a 1:1 curve fitting model was used. Table 3 shows binding and kinetic parameters observed for the indicated modified-IL-2 polypeptide-PEG conjugates with indicated IL-2 receptor subunits. Typical sensorgrams of the binding are shown in FIG. 4.

TABLE 3
Kinetic constants of modified-IL-2 polypeptide-PEG conjugates interacting with IL-2Rα

| | Kₒₙ (M⁻¹S⁻¹) | K_{off} (S⁻¹) | K_{D} (M) |
|---|---|---|---|
| rhIL-2 | 7.32 × 10⁵ | 1.18 × 10⁻² | 1.61E−08 |
| Y31C − PEG20 | 6.15 × 10⁵ | 1.52 × 10⁻² | 2.47E−08 |
| Y31C − | 4.74 ± 0.09 × 10⁵ | 4.31 ± 0.03 × 10⁻³ | 9.09E−09 |
| PEG20 + V69A + Q74P | | | |
| Y31C − | 4.24 × 10⁵ | 3.88 × 10⁻² | 9.15E−08 |
| PEG20 + L18M + L19S | | | |
| Y31C − PEG20 + | 5.81 × 10⁵ | 0.43 × 10⁻² | 7.38E−09 |
| L18M + L19S + V69A + | | | |
| Q74P | | | |
| Y31C − PEG20 + | 3.55 × 10⁵ | 0.32 × 10⁻³ | 9.07E−09 |
| V69A + Q74P + V91K | | | |
| Y31C − PEG20 + N30G | 3.79 × 10⁵ | 2.32 × 10⁻² | 6.10E−08 |
| Y31C − PEG20 + N30S | 6.39 × 10⁵ | 3.02 × 10⁻² | 4.73E−08 |
| Y31C − PEG20 + N30A | 5.20 × 10⁵ | 1.94 × 10⁻² | 3.73E−08 |
| Y31C − PEG20 + N30G | 3.79 × 10⁵ | 2.32 × 10⁻² | 6.10E−08 |
| Y31C − PEG20 + | 3.93 × 10⁵ | 0.35 × 10⁻² | 8.86E−09 |
| V69A + Q74P + I128T | | | |
| Y31C − PEG20 + | 3.18 × 10⁵ | 0.29 × 10⁻² | 9.14E−08 |
| V69A + Q74P + N30S | | | |
| Y31C − | 3.45 × 10⁵ | 0.37 × 10⁻² | 1.06E−08 |
| PEG20 + V69A + Q74P + | | | |
| N88R | | | |
| N29C − PEG20 | 5.38 × 10⁵ | 10.1 × 10⁻² | 1.88E−07 |

Note:
ND = not detectable.

### Example 5. Surface Binding of IL-2 Muteins to IL-2Rαβγ Expressing Cells

CTLL2 cells and IL-2Rα+ T cells, which both express IL-2Rαβγ, were used to analyze surface binding of I1-2 muteins to IL-2Rαβγ.

Activated human T cells isolated from PBMC were generated by incubation with anti-CD3/CD28 Dynabeads. At least 90% of the activated cells were positive for IL-2Rα. The CTLL2 cells and IL-2Rα+ T cells were collected and resuspended in cold binding buffer (FBB, 5% FBS in DPBS) at 2-4 million cells/ml. His-tagged IL-2 and IL-2 mutants were added to the cell suspension, mixed, and incubated at 4° C. for 40 min. The cells were washed once in wash buffer (FWB, 1% FBS in DPBS) and cell pellets were resuspended in FBB with 1:100 anti-His-APC (BioLegend 362605). Samples were incubated at room temperature for 15 min. Cells were then washed with 120 ul FWB, then resuspend for flow cytometry analysis.

As indicated in FIG. 4A and FIG. 4B, several muteins, such as Y31C and Y31C-PEG20, showed enhanced binding relative to erIL-2 to either or both of CTLL2 cells and IL-2Rα positive human T cells. Additionally, ACT5211 (Y31C-PEG20+L18M+L19S) and ACT5261 displayed better binding to IL-2Rα expressing T cells compared to rhIL-2 (FIG. 4C).

### Example 6. Binding of IL-2 Muteins to IL-2Rs in ELISA Assay

To assess the relative binding affinities of exemplary IL-2 muteins for IL-2Rα and IL-2Rβγ compared to the binding affinity of rhIL-2 for these receptor complexes, binding of purified IL-2 muteins to IL-2Rα or IL-2Rβγ receptors was determined by an ELISA-based assay. IL-2Rα and IL-2R βγ-human Fc fusion protein constructs (ACRO Biosystems) were each expressed and immobilized by anti-Human IgG Fc (abeam), which were then captured on the surface of wells of a microplate. Then serial-diluted His-conjugated IL-2, His-conjugated IL-2 mutein (ACT5210), and PEG-conjugated IL-2 mutein (ACT5211) samples were each added to wells containing either IL-2Rα or IL-2Rβγ. Binding was detected using anti-His or anti-PEG antibody conjugated to horse radish peroxidase (HRP) (Genscript), which was incubated with substrate. The quantification of binding was measured with an absorbance-based microplate reader. Data were analyzed by Graphpas prism9 Software. EC50 was calculated for each binding reaction and used as a measure of the relative binding affinities of the IL-2 polypeptides (e.g., rh-IL2, modified IL-2 muteins, and PEGylated versions of same).

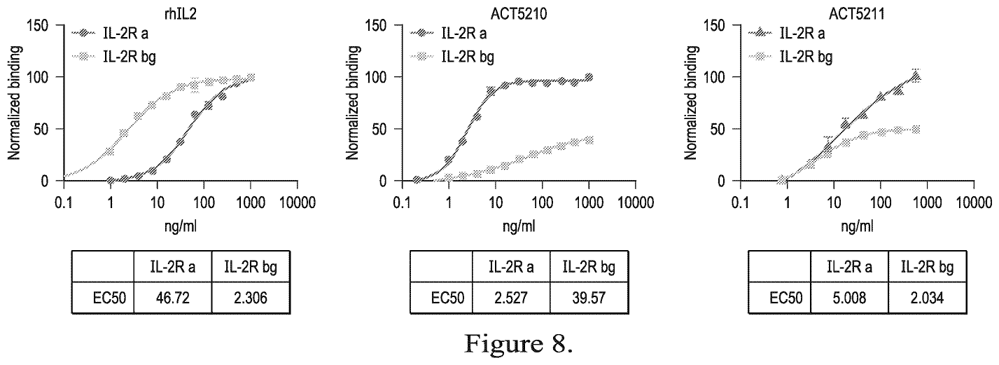

As indicated in FIG. 8, whereas rhIL-2 was observed to display enhanced binding to IL-2Rβγ compared to IL-2Rα. Additionally, rhIL-2 was observed to possess approximately 13-fold lower (i.e, preferential) EC50 value when incubated with IL-2Rβγ compared to the EC50 value obtained upon incubation with IL-2Rα, indicated significantly preferential binding affinity for IL-2Rβγ compared to IL-2Rα. In contrast, both ACT5210 and ACT5211 displayed enhanced binding to IL2Rα and reduced binding on IL2Rβγ. As provided in Table 4 below, the α/βγ EC50 ratios calculated from the measured EC50 values depicted in FIG. 8 for ACT5211 was enhanced over 67-fold compared to the α/βγ EC50 ratio observed for rhIL-2.

TABLE 4
Observed EC50 for binding of rIL-2 and exemplary
muteins to IL-2 IL2R α, and IL2R βγ

| EC50 (ng/ml) | IL-2Rα | IL-2Rβγ | Ratio (α/βγ) |
|---|---|---|---|
| rhIL2 | 46.7 | 2.3 | 20.3 |
| ACT5210 | 2.5 | 39.6 | 0.06 |
| ACT5211 | 5.1 | 21.7 | 0.3 |
| rhIL2/ACT5211 | 6.8 | 0.1 | 67.7 |

### Example 7. Half-Life of IL-2 and Muteins Co-Cultured with T Cells

To assess the relative half-lives of exemplary IL-2 muteins, activated T cells were obtained from thawed human PBMC that were grown in AIM V plus 5% FBS and stimulated with 100 ng/ml anti-CD3 (clone OKT3) for 2 days. The activated T cells were seeded in wells of a 96-well plate and dosed with different concentrations of human recombinant IL-2 (rhIL-2) or the exemplary muteins. Supernatants were collected at 1, 2, 4, 6, 8, 24, 48, 72, 96, 120, 144, 168, and 192 hours, and frozen down promptly after collection. After all samples were collected, frozen supernatants were thawed and tested for IL-2 and IL-2 mutein concentrations using the IL-2 Human Uncoated ELISA Kit (ThermoFisher). Wells that included culture medium lacking IL-2 were used as baseline to evaluate IL-2 stability and were subtracted from samples where IL-2 was added to evaluate IL-2 turnover.

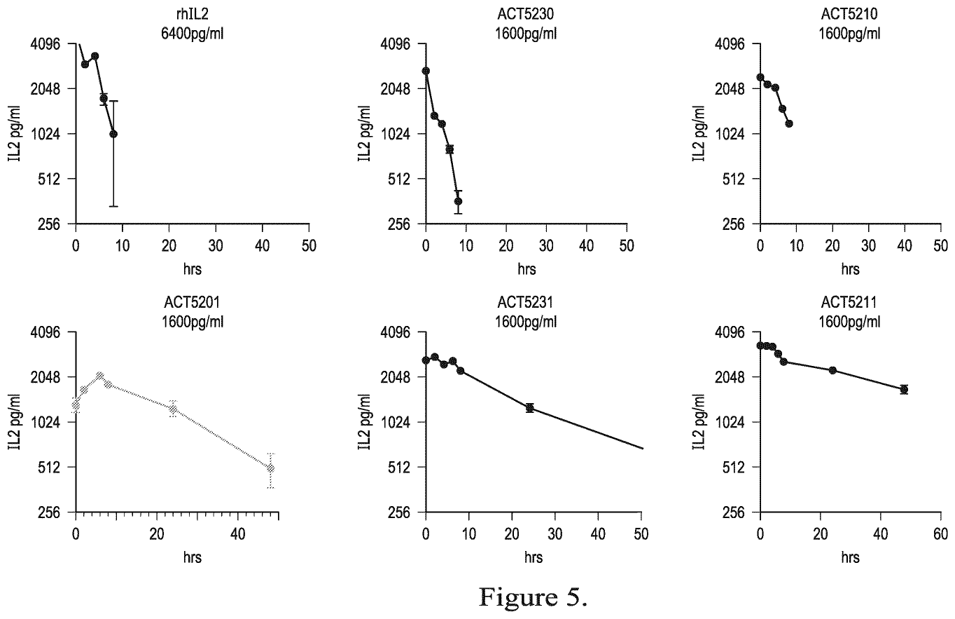

As indicated in FIG. 5, both ACT5210 (Y31C+L18M+L19S) and ACT5230 (Y31C+V69A+Q74P) muteins had decreased turnover rates relative to rhIL-2. Additionally, PEGylated version of these muteins (i.e., (ACT5211 and ACT5231, respectively), resulted in markedly enhanced extension relative to the corresponding non-PEGylated versions. Furthermore, another exemplary PEGylated mutein, ACT5201, was observed to display similarly enhanced half-life extension relative to rhIL-2.

### Example 8. Lymphocyte Proliferation in Response to Pegylated IL-2 Muteins

To assess the ability of exemplary IL-2 muteins to induce lymphocyte proliferation, samples of human PBMCs were thawed and grown to concentrations of approximately 5 million cells/ml in AIM V, 5% FBS, 5 ng/ml anti-CD3 (clone OKT3) in the presence of either rhIL-2 one of each of the exemplary muteins. Starting from day 5, cells were split every 3-4 days with media plus an IL-2 or IL-2 mutein refresher. Starting on day 7, at every 2-3 days, cells were stained with 1:1000 live/dead FITC, 1:200 anti-CD3 BV650, 1:200 anti-CD4 BV421, 1:100 anti-CD25 APC-Cy7, 1:50 anti-FoxP3 Alexa647 and 1:200 anti-CD8 PE. The number of total cells, T regulatory (Treg) cells (CD3+CD4+CD25+Foxp3+) and CD8 T cells (CD3+CD8+) were counted.

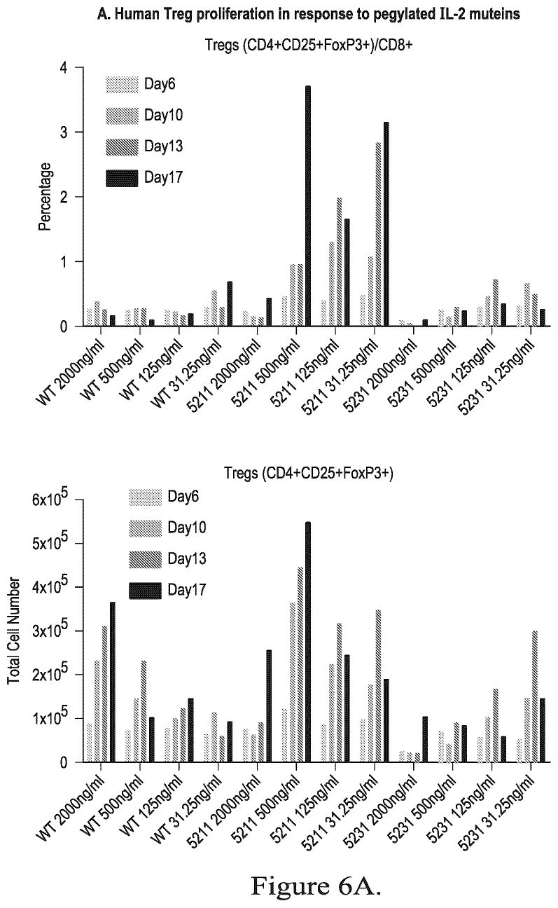

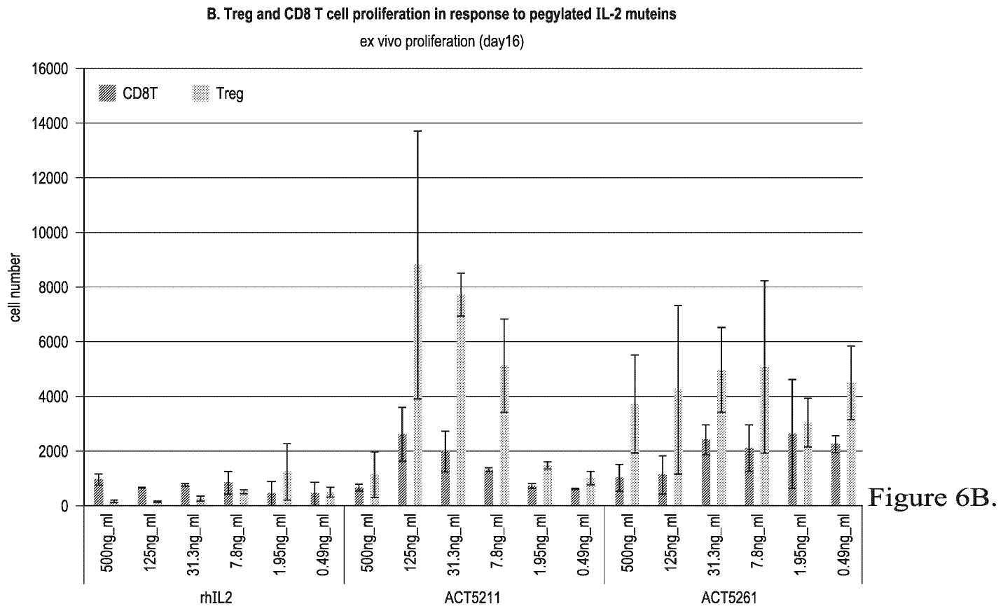

As indicated in FIG. 6A, compared to rhIL-2 (“WT”) the exemplary mutein ACT5211 (Y31C-PEG20+L18M+L19S) preferentially stimulated Tregs over CD8 T cells, especially between days 13 and 17, at concentrations from 1-500 ng/ml. Wild-type recombinant IL-2 and ACT5231 (Y31C-PEG20+V69A+Q74P) didn't show this preferential stimulation of Treg cells. Similarly, in a separate experiment in FIG. 6B, both muteins ACT5211 (Y31C-PEG20+L18M+L19S) and ACT5261 (Y31C-PEG20+L18M+L19S+V69A+Q74P) preferentially stimulated Treg cell proliferation over CD8 T cells, with best effects observed between 10-100 ng/ml.

### Example 9. T Cell Activation as Measured by STAT5 Phosphorylation

To assess the ability of exemplary muteins to activate T cells as reflected by STAT5 phosphorylation, frozen human PBMCs were thawed in AIM V media (ThermoFisher) without serum and incubated at 37° C. for 2-4 h. Following this incubation period, approximately 5×10⁵ cells/well were seeded in a 96-well plate and incubated in the presence of one a selection of exemplary muteins 37° C. for 15 min. After centrifugation, the cell pellets were stained for extracellular markers (1:300-anti-human CD4 FITC, anti-human CD8 APC, anti-human CD25 BV650, anti-human R45RA BV421, BioLegend) and Fixable Viability Dye (1:1000—eFluor 780, ThermoFisher) for 15 min. After washing and fixing in dark for 30 min, the cells were spun and permeabilized with methanol at 4° C. overnight. Cells were then spun-down and stained with 1:80 anti-human pSTAT5-PE (BioLegend) for 30 min at room temperature. The indicated surface markers and the degree of STAT5 phosphorylation were assessed by flow cytometry (NovoCyte, ACEA Biosciences) in naïve CD8+ T cells (CD8+ CD45RA+CD25low, IL-2Rβγ expressing) and Tregs (CD4+CD45RA-CD25high, IL-2Rαβγ expressing).

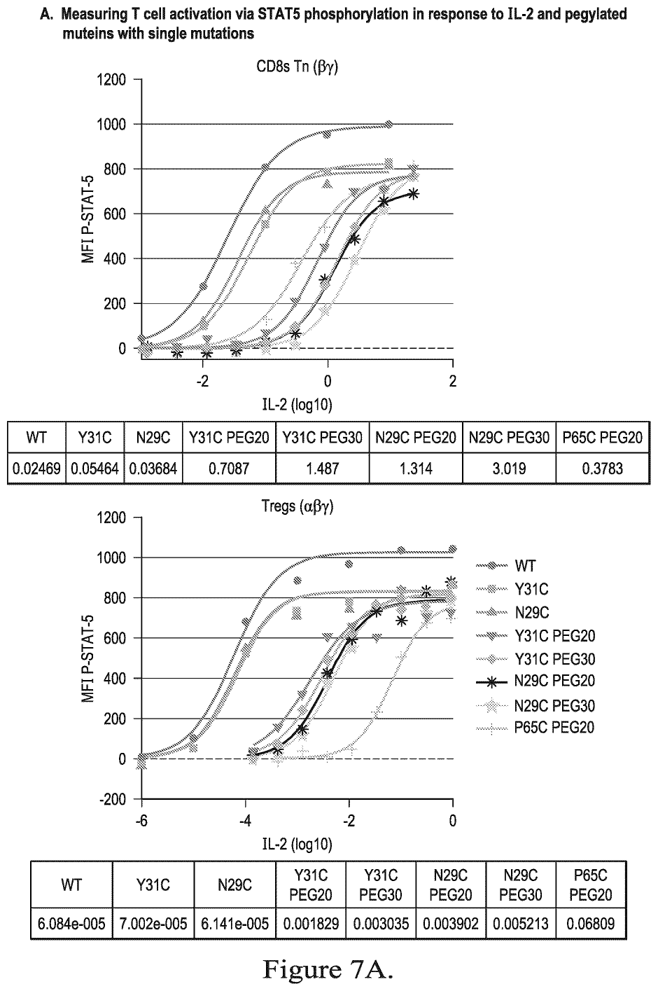

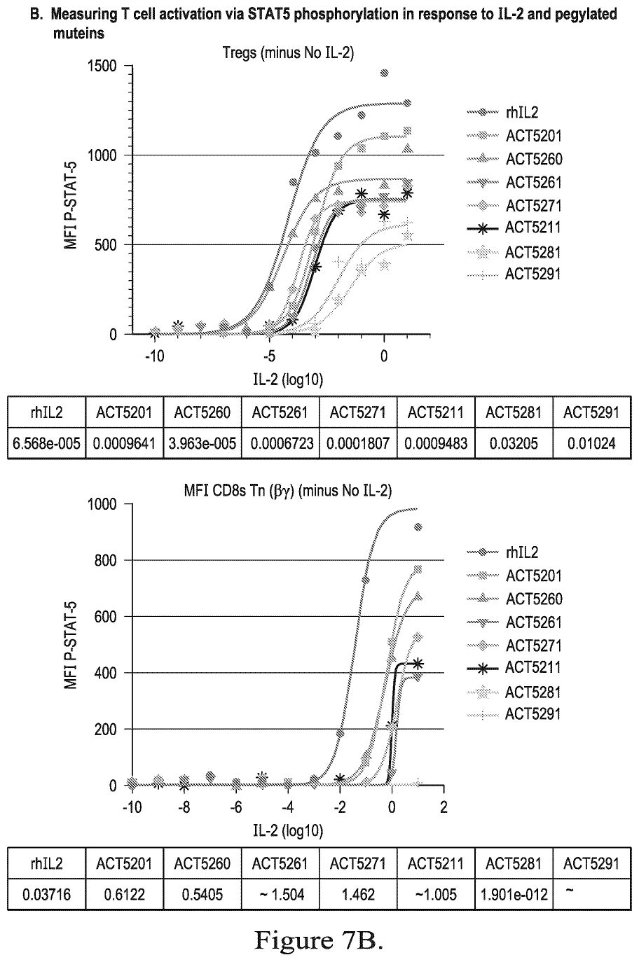

As indicated in FIG. 7A, the dose-response for STAT5 phosphorylation in response to the demonstrated that rhIL-2 and the indicated exemplary IL-2 muteins displayed higher activity (expressed as lower EC50 values, as indicated) when incubated in the presence of IL-2Rαβγ expressing Treg cells than in IL-2Rβγ expressing naïve CD8⁺ T cells. PEGylation of mutein Y31C was observed to slightly reduce the activity observed with the corresponding nonPEGylated mutein in both subtypes of T cells. Additional mutations showed differential effects in regulating Treg or CD8+ naive T cell activation as assessed by STAT5 phosphorylation (see, e.g., FIG. 7B).

### Example 10. PK Study in C57BL/6 Mice

Pharmacokinetics studies of ACT5211 were conducted in C57BL/6 mice. Three mice were used for each blood collection time point. Each mouse was administered with a single subcutaneous dose of 1, 0.3, or 0.1 mg/kg ACT5211. Blood samples were collected at 0.033, 0.083, 0.17, 0.5, 1, 4, 24, 48, 72, 96, 120 and 168 hours post-ACT5211 dose. Blood was allowed to clot at room temperature prior to being centrifugated at 5000 rpm for 10 min. Sera were collected, frozen in dry ice and kept at −80° C. until ELISA analysis.

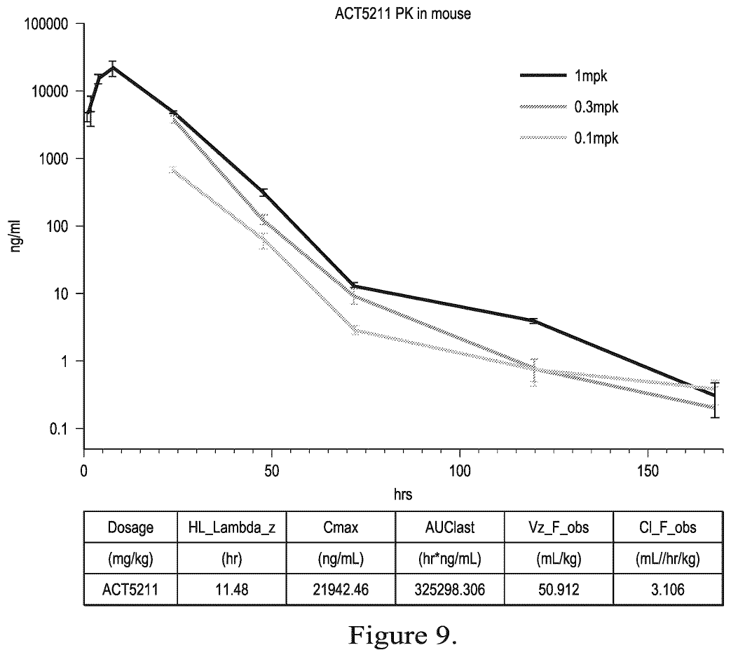

ACT5211 in mouse blood were determined by ELISA assay. The PEG antibody, 5E10E9 (Genscript A01795), was captured on surface of wells of a microplate. Diluted blood serum samples were added into designated wells of coated plate. The samples were detected by biotin conjugated monoclonal IL-2 antibody (BG5, Biotin ThermoFisher) and AvidinHRP (BioLegend), excess detection antibody was washed away, and then HRP conjugate and HRP substrate were added. The ELISA measurements were taken with an absorbance-based microplate reader and was converted to concentrations (ng/ml) using standard curves using corresponding ACT5211 with Graphpas prism9 Software. As depicted in FIG. 9, the serum concentration-time profile of ACT5211 was dose-dependently increase from a peak of from approximately 1000-22,000 ng/ml. Additionally, the observed terminal half-life (t_{1/2}) for ACT5211 was 11.48 hours, approximately 4.6-fold greater than that for rhIL-2 (approximately 2.5 hours) See, e.g., R. Melder et al. Cancer Immunology and Immunotherapy 54(6):535-47 (2005).

### Example 11. PD Study in C57BL/6 Mice

A Pharmacodynamic study of ACT5211 was conducted in C57BL/6 mice. Three mice were used for each blood collection time point. Each mouse was administered with a single subcutaneous dose of 1, 0.3, or 0.1 mg/kg ACT5211. Three days after dosing, mice were sacrificed, and blood and spleen samples were collected.

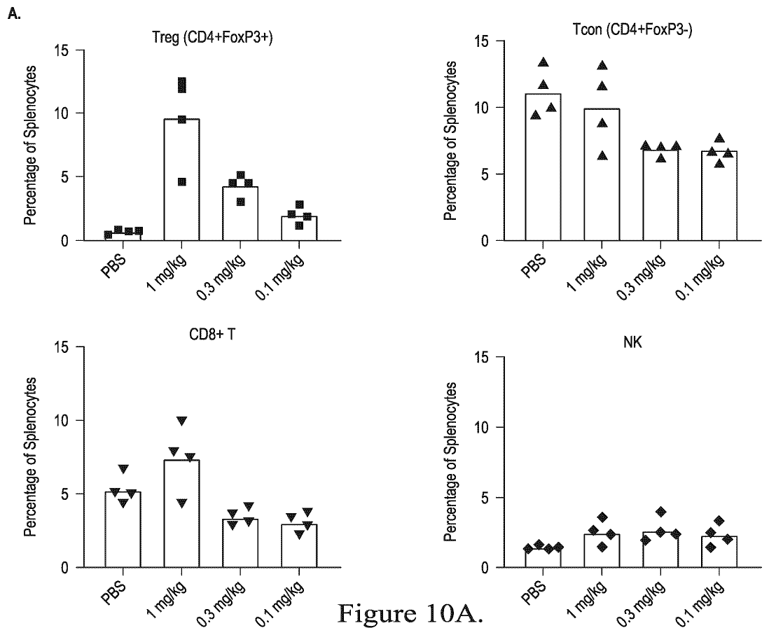

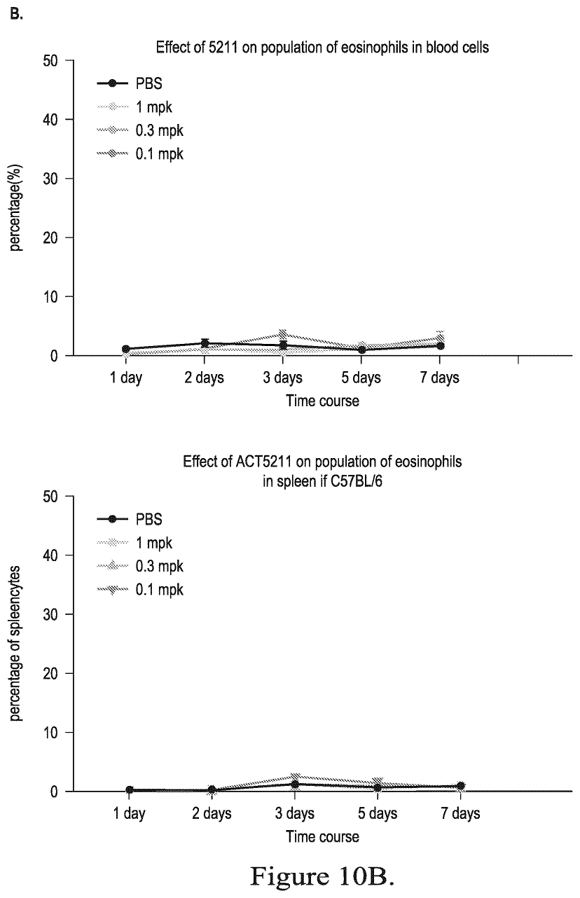

Spenocytes obtained from spleen samples were stained with Fluorophore conjugated anti-CD3, anti-CD4, anti-Foxp3, anti-CD49b and anti-CD8 antibodies (all from Biolegend) and detected by flow cytometry. The percentage of cell Treg, Tcontrol, CD8+ T cells, and NK cells were each calculated as the percentage of splenocytes in the samples. As evident in FIG. 10A, ACT2511 dose-dependently stimulated proliferation of Treg (CD4+FoxP3+) cells, with the lowest dose (0.1 mg/kg) eliciting approximately a four-fold increase in the percentage of such Treg cells, and the highest dose (1 mg/kg) eliciting approximately a 10-fold increase in the percentage of such Treg cells. In contrast, ACT2511 did not elicit significant proliferation of Natural Killer (NK) cells in animals relative to mock-treated (PBS) animals, and modestly induced proliferation of CD8+ T cells, in animals relative to mock-treated (PBS) animals at only the highest dose (i.e., 1 mg/kg). Furthermore, ACT2511, if anything, elicited a decrease in the percentage of Tcon cells in animals relative to mock-treated (PBS) animals.

Eosinophils were also obtained from the spleen and peripheral blood samples were collected at day 1, 2, 3, 5, and 7 post-injection. Eosinophils were determined by flow cytometric analysis using anti-CD45, anti-siglec F and anti-CCR3 antibodies (all from Biolegend). The percentage of eosinophils were each calculated as the percentage of splenocytes in the samples. As evident in FIG. 10B, ACT2511 did not significantly affect the percentage of eosinophils in treated animals at any of the tested time points relative to that observed with mock-treated (PBS) animals.

SEQUENCES
Protein
Artificial
Variant of Homo sapiens IL-2 chain A with
C125S mutation
SEQ ID NO: 1
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFSQSIISTLT
Protein
Artificial
Variant of Homo sapiens IL-2 chain A with
A1M mutation
SEQ ID NO: 2
MPTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT
Protein
Homo sapiens
Interleukin 2, chain A2, mature
(GenBank Accession: AAH66254.1)
SEQ ID NO: 3
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

1. A modified interleukin 2 (IL-2) polypeptide comprising an amino acid sequence having at least 80% identity to SEQ ID NO:1 or SEQ ID NO:2, wherein the modified IL-2 polypeptide comprises a substitution with a natural or unnatural amino acid at a position selected from the group consisting of L18, L19, N29, Y31, V69, N71, Q74, N88, V91, I128 and a combination thereof, wherein said modified IL-2 polypeptide:
a) has enhanced binding to an interleukin 2 receptor α (IL-2Rα) compared to an IL-2 polypeptide without the substitution; and/or
b) has enhanced binding to an interleukin 2 receptor αβγ (IL-2Rαβγ) compared an IL-2 polypeptide without the substitution; and/or
b) has enhanced binding to cells expressing an interleukin 2 receptor αβγ (IL-2Rαβγ) compared to an IL-2 polypeptide without the substitution; and/or
c) has enhanced receptor signaling potency via IL-2Rαβγ compared to an IL-2 polypeptide without the substitution; and/or
e) has an enhanced ratio of IL-2Rαβγ receptor signaling potency to IL-2Rβγ receptor signaling potency compared to the ratio of IL-2Rαβγ receptor signaling potency to IL-2Rβγ receptor signaling potency of an IL-2 polypeptide without the substitution; and/or
f) is configured to be conjugated to a conjugating moiety; and/or
g) is conjugated to a conjugating moiety; and/or
h) combinations of a) through g).

2. The modified IL-2 polypeptide of claim 1, wherein the modified IL-2 polypeptide comprises:
a) a substitution with cysteine, lysine, histidine, arginine, aspartic acid, glutamic acid, serine, threonine, alanine, tryptophan, isoleucine, phenylalanine, or tyrosine at position N29; and/or
b) a substitution with cysteine, lysine, histidine, arginine, aspartic acid, glutamic acid, serine, threonine, alanine, tryptophan, isoleucine, or phenylalanine at position Y31.

3. The modified IL-2 polypeptide of claim 1 or claim 2, wherein the modified IL-2 polypeptide comprises the substitution N29C.

4. The modified IL-2 polypeptide of any of claims 1-3, wherein the modified IL-2 polypeptide comprises the substitution Y31C.

5. The modified IL-2 polypeptide of claims 1-4, wherein the modified IL-2 polypeptide comprises a substitution with lysine, cysteine, histidine, arginine, aspartic acid, glutamic acid, serine, threonine, alanine, methionine, tryptophan, isoleucine, phenylalanine, proline, or tyrosine at one or more positions selected from the group consisting of L18, L19, V69, Q74, N88, V91, and I128.

6. The modified IL-2 polypeptide of any of claims 1-5, wherein the modified IL-2 polypeptide comprises a substitution selected from the group consisting of Y31C.

7. The modified IL-2 polypeptide of any of claims 1-6, wherein the modified IL-2 polypeptide is conjugated to a conjugating moiety selected from the group consisting of a water-soluble polymer, a lipid, a peptide, a protein, a polypeptide, and combinations thereof.

8. The modified IL-2 polypeptide of any of claims 1-7, wherein the modified IL-2 polypeptide is conjugated to a polyethylene glycol.

9. The modified IL-2 polypeptide of any of claims 1-8, wherein the modified IL-2 polypeptide comprises a mutation selected from the group consisting of N29C, N30C, Y31C, E100C, N119C, T123C, S127C, or T131C, wherein the polypeptide is pegylated at the N29C, N30C, Y31C, E100C, N119C, T123C, S127C, or T131C site.

10. The modified IL-2 polypeptide of any of claims 1-9, wherein the modified IL-2 polypeptide comprises a N29C or Y31C mutation.

11. The modified IL-2 polypeptide of any of claims 1-10, wherein the modified IL-2 polypeptide comprises:
a) a substitution with lysine, cysteine, histidine, arginine, aspartic acid, glutamic acid, serine, threonine, alanine, tryptophan, isoleucine, phenylalanine, or tyrosine at a position selected from the group consisting of N29, N30, Y31 and combinations thereof; or
b) a substitution with lysine, cysteine, histidine, arginine, aspartic acid, glutamic acid, serine, threonine, alanine, tryptophan, isoleucine, phenylalanine, or tyrosine at a position selected from the group consisting of N30, Y31, and combinations thereof.

12. The modified IL-2 polypeptide of any of claims 1-11, wherein the modified IL-2 polypeptide comprises:
a) a substitution with a natural amino acid or an unnatural amino acid at one or more positions selected from the group consisting of N29, N30, Y31, and is:
(i) unconjugated;
(ii) conjugated to; or
(iii) configured to be conjugated to;
one or more water-soluble polymers, lipids, proteins, or peptides at one or more positions selected from the group consisting of N29, N30, Y31, E100, N119, T123, S127, T131; and/or
b) a substitution with a natural amino acid or an unnatural amino acid at a position selected from the group consisting of N29, N30, Y31, and is:
(i) unconjugated;
(ii) conjugated to; or
(iii) configured to be conjugated to;
one or more water-soluble polymers, lipids, proteins, or peptides at one or more positions selected from the group consisting of N29, N30, Y31; and/or
c) a substitution with a natural amino acid or an unnatural amino acid at a position selected from the group consisting of N29, N30, Y31 and a combination thereof, and is:
(i) unconjugated;
(ii) conjugated to; or
(iii) configured to be conjugated to;
one or more water-soluble polymers, lipids, proteins, or peptides at the N terminal and/or C terminal of the modified IL-2 polypeptide.

13. The modified IL-2 polypeptide of any of claims 1-12 wherein the modified IL-2 polypeptide comprises:
a) a substitution with cysteine at one or more positions selected from the group consisting of N29, N30, Y31; and/or
b) a substitution with cysteine at one or more positions selected from the group consisting of N30, Y31; and/or
c) comprises a substitution with cysteine at a position of Y31; and/or
f) comprises a substitution with cysteine at a position of N30.

14. The modified IL-2 polypeptide of any of claims 1-13, wherein the modified IL-2 polypeptide comprises one or more substitutions with a natural amino acid or an unnatural amino acid at a position within IL-2Rα interaction region, and/or IL-2Rβ interaction region and/or IL-2Rγ interaction region.

15. The modified IL-2 polypeptide of any of claims 1-14, wherein the modified IL-2 polypeptide comprises one or more substitutions with a natural amino acid or an unnatural amino acid at a position within IL-2Rβ interaction region and/or IL-2Rγ interaction region.

16. The modified IL-2 polypeptide of any of claims 1-15, wherein the modified IL-2 polypeptide comprises one or more substitutions with a natural amino acid or an unnatural amino acid at a position selected from the group consisting of L18, L19, V69, Q74, N88, V91, I128, and a combination thereof.

17. The modified IL-2 polypeptide of any of claims 1-16, wherein the modified IL-2 polypeptide comprises one or more substitutions with lysine, cysteine, histidine, arginine, aspartic acid, glutamic acid, serine, threonine, alanine, methionine, tryptophan, isoleucine, phenylalanine, proline, or tyrosine at a position selected from the group consisting of L18, L19, V69, Q74, N88, V91, I128, and a combination thereof.

18. The modified IL-2 polypeptide of any of claims 1-17, wherein the modified IL-2 polypeptide comprises:
a) a substitution with methionine at a position L18; and/or
b) a substitution with serine at a position of L19; and/or
c) a substitution with cysteine at position of Y31, and/or
d) comprises a substitution with alanine at a position of V69; and/or
e) comprises a substitution with proline at a position of Q74; and/or
f) comprises a substitution with arginine, aspartic acid, glutamic acid, lysine at a position of N88; and/or
g) comprises a substitution with arginine at a position of N88; and/or
h) comprises a substitution with aspartic acid at a position of N88;
i) comprises a substitution with glutamic acid at a position of N88;
j) comprises a substitution with lysine at a position of N88;
k) comprises a substitution with lysine at a position of V91;
l) comprises a substitution with threonine at a position of I128; and/or
m) combinations of a) through 1).

19. The modified IL-2 polypeptide of any of claims 1-18, wherein the modified IL-2 polypeptide comprises:
a) a substitution with a natural amino acid at a position within IL-2Rα interaction region and a substitution with a natural amino acid at a position within IL-2Rβ interaction region; and/or
b) a substitution with a natural amino acid at a position within IL-2Rα interaction region and a substitution with a natural amino acid at a position within IL-2Rγ interaction region; and/or
c) a substitution with a natural amino acid at a position within IL-2Rα interaction region, a substitution with a natural amino acid at a position within IL-2Rβ interaction region and a substitution with a natural amino acid at a position within IL-2Rγ interaction region.

20. The modified IL-2 polypeptide of any of claims 1-19, wherein the modified IL-2 polypeptide has increased binding to an IL-2Rα and/or IL-2Rαβγ compared to an IL-2 polypeptide comprising an amino acid sequence set forth in SEQ ID NO:1 or SEQ ID NO:2 without the substitution.

21. The modified IL-2 polypeptide of any of claims 1-20, wherein the binding affinity of the modified IL-2 polypeptide to an IL-2Rα and/or IL-2Rαβγ is increased from about 10% to about 100%, or is increased from about 1-fold to about 100,000-fold or more.

22. The modified IL-2 polypeptide of any of claims 1-21, wherein the modified IL-2 polypeptide has increased binding to IL-2Rα expressing cells and/or IL-2Rαβγ expressing cells compared to an IL-2 polypeptide comprising an amino acid sequence set forth in SEQ ID NO:1 or SEQ ID NO:2 without the substitution.

23. The modified IL-2 polypeptide of any of claims 1-22, wherein the modified IL-2 polypeptide has increased binding to IL-2Rα expressing cells and/or IL-2Rαβγ expressing cells that is increased from about 10% to about 100%, or is increased from about 1-fold to about 100,000-fold or more.

24. The modified IL-2 polypeptide of any of claims 1-23, wherein the modified IL-2 polypeptide has reduced internalization by IL-2Rα expressing cells and/or IL-2Rαβγ expressing cells compared to an IL-2 polypeptide comprising an amino acid sequence set forth in SEQ ID NO:1 or SEQ ID NO:2 without the substitution.

25. The modified IL-2 polypeptide of any of claims 1-24, wherein the modified IL-2 polypeptide has internalization by IL-2Rα expressing cells and/or IL-2Rαβγ expressing cells that is from about 10% to about 100%, or is increased from about 1-fold to about 100,000-fold or more.

26. The modified IL-2 polypeptide of any of claims 1-25, wherein the modified IL-2 polypeptide has no detectable internalization by IL-2Rα expressing cells and/or IL-2Rαβγ expressing cells.

27. The modified IL-2 polypeptide of any of claims 1-26, wherein the modified IL-2 polypeptide has increased receptor signaling potency to IL-2Rαβγ compared to an IL-2 polypeptide comprising an amino acid sequence set forth in SEQ ID NO:1 or SEQ ID NO:2 without the substitution.

28. The modified IL-2 polypeptide of any of claims 1-27, wherein the modified IL-2 polypeptide has increased binding to an IL-2Rα compared to an IL-2 polypeptide comprising an amino acid sequence set forth in SEQ ID NO:1 or SEQ ID NO:2 without the substitution and has increased receptor signaling potency to IL-2Rαβγ compared to an IL-2 polypeptide comprising an amino acid sequence set forth in SEQ ID NO:1 or SEQ ID NO:2 without the substitution.

29. The modified IL-2 polypeptide of any of claims 1-28, wherein the modified IL-2 polypeptide has increased binding to an IL-2Rα and/or IL-2Rαβγ, and increased binding on IL-2Ra expressing cells and/or IL-2Rαβγ expressing cells compared an IL-2 polypeptide comprising an amino acid sequence set forth in SEQ ID NO:1 or SEQ ID NO:2 without the substitution, and has reduced internalization by IL-2Rα expressing cells and/or IL-2Rαβγ expressing cells compared to an IL-2 polypeptide comprising an amino acid sequence set forth in SEQ ID NO:1 or SEQ ID NO:2 without the substitution.

30. The modified IL-2 polypeptide of any of claims 1-29, wherein the modified IL-2 polypeptide has: (i) increased binding to an IL-2Rα and/or IL-2Rαβγ; (ii) increased binding on IL-2Rα expressing cells and/or IL-2Rαβγ expressing cells compared to an IL-2 polypeptide comprising an amino acid sequence set forth in SEQ ID NO:1 or SEQ ID NO:2 without the substitution; (iii) no detectable internalization by IL-2Rα expressing cells and/or IL-2Rαβγ expressing cells compared to an IL-2 polypeptide comprising an amino acid sequence set forth in SEQ ID NO:1 or SEQ ID NO:2 without the substitution; (iv) and increased receptor signaling potency to IL-2Rαβγ compared to an IL-2 polypeptide comprising an amino acid sequence set forth in SEQ ID NO:1 or SEQ ID NO:2 without the substitution.

31. The modified IL-2 polypeptide of any of claims 1-30, wherein the modified IL-2 polypeptide has:
reduced binding level to an interleukin 2 receptor β (IL-2Rβ) or an interleukin 2 receptor γ (IL-2Rγ) compared to an IL-2 polypeptide comprising an amino acid sequence set forth in SEQ ID NO:1 or SEQ ID NO:2 without the substitution;
and/or reduced receptor signaling potency to IL-2Rβγ compared to an IL-2 polypeptide comprising an amino acid sequence set forth in SEQ ID NO:1 or SEQ ID NO:2 without the substitution.

32. The modified IL-2 polypeptide of any of claims 1-31, wherein the modified IL-2 polypeptide has lower receptor signaling potency to IL-2Rβγ compared to an IL-2 polypeptide comprising an amino acid sequence set forth in SEQ ID NO:1 or SEQ ID NO:2 without the substitution.

33. The modified IL-2 polypeptide of any of claims 1-32, wherein the modified IL-2 polypeptide has: (i) lower binding level to an IL-2Rβ or an IL-2Rγ compared to an IL-2 polypeptide comprising an amino acid sequence set forth in SEQ ID NO:1 or SEQ ID NO:2 without the substitution; and (ii) lower receptor signaling potency to IL-2Rβγ compared to an IL-2 polypeptide comprising an amino acid sequence set forth in SEQ ID NO:1 or SEQ ID NO:2 without the substitution.

34. The modified IL-2 polypeptide of any of claims 1-33, wherein the modified IL-2 polypeptide has increased ratio between its signaling potency to IL-2Rαβγ and the signaling potency to IL-2Rβγ compared to an IL-2 polypeptide comprising an amino acid sequence set forth in SEQ ID NO:1 or SEQ ID NO:2 without the substitution.

35. The modified IL-2 polypeptide of any of claims 1-34, wherein the modified IL-2 polypeptide has increased ratio between its signaling potency to IL-2Rαβγ and the signaling potency to IL-2Rβγ is more than 1-fold, more than 10-fold, more than 100 fold, more than 1,000-fold, more than 10,000-fold, more than 10,0000-fold.

36. The modified IL-2 polypeptide of any of claims 1-35, wherein the modified IL-2 polypeptide comprises an N terminal deletion, wherein said deletion comprises a deletion of one or more of amino acid residues 1 through 30, inclusive, that are present in the corresponding IL-2 modified polypeptide that does not comprise said N-terminal deletion.

37. The modified IL-2 polypeptide of any of claims 1-36, wherein the modified IL-2 polypeptide comprises a C terminal deletion, wherein said deletion comprises a deletion of one or more of amino acid residues 114 through 134, inclusive, that are present in the corresponding IL-2 modified polypeptide that does not comprise said C-terminal deletion.

38. The modified IL-2 polypeptide of any of claims 1-37, wherein the modified IL-2 polypeptide comprises a N terminal deletion and a C terminal deletion.

39. The modified IL-2 polypeptide of any of claims 1-38, wherein the modified IL-2 polypeptide is a part of a fusion polypeptide comprising an additional amino acid sequence.

40. The modified IL-2 polypeptide of any of claims 1-39, wherein the modified IL-2 polypeptide comprises a recombinant fusion protein comprising the modified IL-2 polypeptide and an additional amino acid sequence.

41. The modified IL-2 polypeptide of any of claims 1-40, wherein the N terminus or the C terminus of the modified IL-2 polypeptide is fused to an additional amino acid sequence.

42. The modified IL-2 polypeptide of any of claims 1-41, wherein the N terminus or the C terminus of the modified IL-2 polypeptide is fused to an additional amino acid sequence, wherein said additional amino acid sequence comprises an antibody sequence or a portion or a fragment thereof.

43. The modified IL-2 polypeptide of any of claims 1-42, wherein the N terminus or the C terminus of the modified IL-2 polypeptide is fused to an additional amino acid sequence, wherein said additional amino acid sequence comprises an Fc portion of an antibody or a portion or a fragment thereof.

44. The modified IL-2 polypeptide of any of claims 1-43, wherein the modified IL-2 polypeptide is isolated.

45. The modified IL-2 polypeptide of any of claims 1-44, wherein the modified IL-2 polypeptide is expressed from a vector comprising a polynucleotide sequence that encodes the modified IL-2 polypeptide.

46. The modified IL-2 polypeptide of any of claims 1-45, wherein the modified IL-2 polypeptide is expressed from a vector comprising a polynucleotide sequence that encodes the modified IL-2 polypeptide, wherein said vector is an RNA vector, a DNA, a viral vector, or a non-viral vector.

47. A modified IL-2 polypeptide, which comprises a modified IL-2 polypeptide of any of claims 1-46 that is conjugated to a water-soluble polymer, a lipid, a polypeptide, a protein, or a peptide.

48. The modified IL-2 polypeptide of any of claims 1-47, wherein the modified IL-2 polypeptide is conjugated to one or more water-soluble polymers, lipids, proteins, or peptides through one or more covalent bonds.

49. The modified IL-2 polypeptide of any of claims 1-48, wherein the modified IL-2 polypeptide is conjugated to one or more water-soluble polymers, lipids, proteins, or peptides through one or more non-covalent bonds.

50. The modified IL-2 polypeptide of any of claims 1-49, wherein the modified IL-2 polypeptide is conjugated to a water-soluble polymer, a lipid, a protein, or a peptide via a substituted natural amino acid or unnatural amino acid at a position selected from the group consisting of L18, L19, N30, Y31, V69, Q74, N88, V91, I128, E100, N119, T123, S127, T131, and combinations thereof.

51. The modified IL-2 polypeptide any of claims 1-50, wherein the modified IL-2 polypeptide is conjugated to a water-soluble polymer, a lipid, a protein, or a peptide via a substituted natural amino acid at a position selected from the group consisting of L18, L19, N30, Y31, V69, Q74, N88, V91, I128, E100, N119, T123, S127, T131, and combinations thereof.

52. The modified IL-2 polypeptide any of claims 1-51, wherein the modified IL-2 polypeptide is conjugated to a water-soluble polymer, a lipid, a protein, or a peptide via a substituted lysine, cysteine, histidine, arginine, aspartic acid, glutamic acid, serine, threonine, alanine, tryptophan, isoleucine, phenylalanine, or tyrosine at a position selected from the group consisting of L18, L19, N30, Y31, V69, Q74, N88, V91, I128, E100, N119, T123, S127, T131, and combinations thereof.

53. The modified IL-2 polypeptide of any of claims 1-52, wherein the modified IL-2 polypeptide is conjugated to a water-soluble polymer, a lipid, a protein, or a peptide via a substituted cysteine at a position selected from the group consisting of L18, L19, N30, Y31, V69, Q74, N88, V91, I128, E100, N119, T123, S127, T131, and combinations thereof.

54. The modified IL-2 polypeptide of any of claims 1-53, wherein the modified IL-2 polypeptide is conjugated to a water-soluble polymer, a lipid, a protein, or a peptide via a substituted natural amino acid or unnatural amino acid at a position selected from the group consisting of L18, L19, N30, Y31, V69, Q74, N88, V91, I128, E100, N119, T123, S127, T131, and combinations thereof.

55. The modified IL-2 polypeptide any of claims 1-54, wherein the modified IL-2 polypeptide is conjugated to a water-soluble polymer, a lipid, a protein, or a peptide via a substituted natural amino acid at a position selected from the group consisting of L18, L19, N30, Y31, V69, Q74, N88, V91, I128, E100, N119, T123, S127, T131, and combinations thereof.

56. The modified IL-2 polypeptide of claims 1-55, wherein the modified IL-2 polypeptide is conjugated to a water-soluble polymer, a lipid, a protein, or a peptide via a substituted lysine, cysteine, histidine, arginine, aspartic acid, glutamic acid, serine, threonine, alanine, tryptophan, isoleucine, phenylalanine, or tyrosine at a position selected from the group consisting of L18, L19, N30, Y31, V69, Q74, N88, V91, I128, E100, N119, T123, S127, T131, and combinations thereof.

57. The modified IL-2 polypeptide of claims 1-56, wherein the modified IL-2 polypeptide is conjugated to a water-soluble polymer, a lipid, a protein, or a peptide via a substituted cysteine at a position selected from the group consisting of L18, L19, N30, Y31, V69, Q74, N88, V91, I128, E100, N119, T123, S127, T131, and combinations thereof.

58. The modified IL-2 polypeptide of any of claims 1-57, wherein the modified IL-2 polypeptide is conjugated to a water-soluble polymer, a lipid, a protein, or a peptide via a single amino acid residue of the modified IL-2 polypeptide.

59. The modified IL-2 polypeptide of any of claims 1-58, wherein the modified IL-2 polypeptide is conjugated to a water-soluble polymer, a lipid, a protein, or a peptide via:
i) the alpha amino group of the N-terminal amino acid residue of the modified IL-2 polypeptide;
ii) the epsilon amino group of a lysine amino acid residue of the modified IL-2 polypeptide; or
iii) an N-glycosylation site or O-glycosylation site of the modified IL-2 polypeptide.

60. The modified IL-2 polypeptide of any of claims 1-59, wherein the modified IL-2 polypeptide is covalently conjugated to a water-soluble polymer, a lipid, a protein, or a peptide through a linker.

61. The modified IL-2 polypeptide of any of claims 1-60, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence.

62. The modified IL-2 polypeptide of any of claims 1-61, wherein the modified IL-2 polypeptide is conjugated to a water-soluble polymer, a lipid, a protein, or a peptide via a single amino acid residue located within the modified IL-2 polypeptide.

63. The modified IL-2 polypeptide of any of claims 1-62, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the single amino acid residue is located within the additional amino acid sequence.

64. The modified IL-2 polypeptide of any of claims 1-63, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the additional amino acid sequence comprises an antibody sequence or a portion or a fragment thereof.

65. The modified IL-2 polypeptide of any of claims 1-64, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the additional amino acid sequence comprises a Fc portion of an antibody.

66. The modified IL-2 polypeptide of any of claims 1-65, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the single amino acid residue is:
i) the alpha amino group of the N-terminal amino acid residue of the fusion polypeptide;
ii) the epsilon amino group of a lysine amino acid residue of the fusion polypeptide; or
iii) an N-glycosylation site or O-glycosylation site of the fusion polypeptide.

67. The modified IL-2 polypeptide of any of claims 1-65, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence, wherein the fusion polypeptide is covalently conjugated to the water-soluble polymer, a lipid, a protein, or a peptide through a linker.

68. The modified IL-2 polypeptide of any of claims 1-67, wherein the modified IL-2 polypeptide is conjugated to a water-soluble polymer.

69. The modified IL-2 polypeptide of any of claims 1-68, wherein the modified IL-2 polypeptide is conjugated to a water-soluble polymer comprising polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or combinations thereof.

70. The modified IL-2 polypeptide of any of claims 1-69, wherein the modified IL-2 polypeptide is conjugated to a water-soluble polymer comprising a PEG molecule.

71. The modified IL-2 polypeptide of any of claims 1-70, wherein the modified IL-2 polypeptide is conjugated to a water-soluble polymer comprising a linear PEG molecule.

72. The modified IL-2 polypeptide of any of claims 1-71, wherein the modified IL-2 polypeptide is conjugated to a water-soluble polymer comprising a branched PEG molecule.

73. The modified IL-2 polypeptide of any of claims 1-72, wherein the modified IL-2 polypeptide is conjugated to a water-soluble polymer comprising a branched PEG molecule comprising about three to about ten PEG chains emanating from a central core group.

74. The modified IL-2 polypeptide of any of claims 1-73, wherein the modified IL-2 polypeptide is conjugated to a water-soluble polymer comprising a branched PEG molecule, wherein the branched PEG molecule is a star PEG comprising from about 10 to about 100 PEG chains emanating from a central core group.

75. The modified IL-2 polypeptide of any of claims 1-74, wherein the modified IL-2 polypeptide is conjugated to a water-soluble polymer comprising a branched PEG molecule, wherein the branched PEG molecule is a comb PEG comprising multiple PEG chains grafted onto a polymer backbone.

76. The modified IL-2 polypeptide of any of claims 1-75, wherein the modified IL-2 polypeptide is conjugated to a water-soluble polymer comprising a PEG molecule, wherein the PEG molecule has a range of molecular weight from about 300 g/mol to about 10,000,000 g/mol.

77. The modified IL-2 polypeptide of any of claims 1-76, wherein the modified IL-2 polypeptide is conjugated to a water-soluble polymer comprising a PEG molecule, wherein the PEG molecule has an average molecular weight from about 5,000 Daltons to about 1,000,000 Daltons.

78. The modified IL-2 polypeptide of any of claims 1-77, wherein the modified IL-2 polypeptide is conjugated to a water-soluble polymer comprising a PEG molecule, wherein the PEG molecule has an average molecular weight of from about 20,000 Daltons to about 30,000 Daltons.

79. The modified IL-2 polypeptide of any of claims 1-78, wherein the modified IL-2 polypeptide is conjugated to a water-soluble polymer comprising a PEG molecule, wherein the PEG molecule is a monodisperse, uniform, or discrete PEG molecule.

80. The modified IL-2 polypeptide of any of claims 1-79, wherein the modified IL-2 polypeptide is conjugated to a water-soluble polymer, wherein the water-soluble polymer comprises a polysaccharide.

81. The modified IL-2 polypeptide of any of claims 1-80, wherein the modified IL-2 polypeptide is conjugated to a lipid.

82. The modified IL-2 polypeptide of any of claims 1-81, wherein the modified IL-2 polypeptide is conjugated to a lipid, wherein the lipid comprises a fatty acid.

83. The modified IL-2 polypeptide of any of claims 1-82, wherein the modified IL-2 polypeptide is conjugated to a protein.

84. The modified IL-2 polypeptide of any of claims 1-83, wherein the modified IL-2 polypeptide is conjugated to a protein, wherein the protein comprises an antibody or a binding fragment thereof.

85. The modified IL-2 polypeptide of any of claims 1-84, wherein the modified IL-2 polypeptide is conjugated to an Fc portion of an antibody or a fragment thereof.

86. The modified IL-2 polypeptide of any of claims 1-85, wherein the modified IL-2 polypeptide is conjugated to a water-soluble polymer, a lipid, a protein, or a peptide that is indirectly bound to the substituted natural amino acid or unnatural amino acid of the modified IL-2 polypeptide through a linker.

87. The modified IL-2 polypeptide of any of claims 1-86, wherein the modified IL-2 polypeptide is conjugated to a water-soluble polymer, a lipid, a protein, or a peptide that is directly bound to the substituted natural amino acid or unnatural amino acid of the modified IL-2 polypeptide.

88. The modified IL-2 polypeptide of any of claims 1-87, wherein the modified IL-2 polypeptide, wherein the modified IL-2 polypeptide has a half-life in vivo from about 5 minutes to about 10 days.

89. The modified IL-2 polypeptide of any of claims 1-88, wherein the modified IL-2 polypeptide is selected from the group consisting of ACT5200, ACT5201, ACT5210, ACT5211, ACT5212, ACT522S0, ACT522S1, ACT5230, ACT5231, ACT5260, ACT5261, ACT5270, ACT5271, ACT5280, ACT5281, ACT5290, and ACT5291.

90. A pharmaceutical composition comprising an effective amount of a modified IL-2 polypeptide of any of claims 1-89 and a pharmaceutically acceptable carrier or excipient.

91. The pharmaceutical composition of claim 90, wherein the pharmaceutical composition further comprises another active ingredient.

92. The pharmaceutical composition of claim 90 or claim 91, further comprising one or more additional ingredients, wherein the one or more active ingredients comprises:
(i) an anti-inflammatory substance or an anti-autoimmune substance;
(ii) an anti-neoplasm substance;
(iii) an anti-infectious disease substance; and/or
(iv) an immune deficiency disorder.

93. The modified IL-1 polypeptide of any of claims 1-89 or the pharmaceutical composition of any of claims 90-92 for use in treating or preventing a disease or disorder in a subject having, or suspected of having, the disease or disorder.

94. The modified IL-1 polypeptide of any of claims 1-89 or the pharmaceutical composition of any of claims 90-92 for use in treating or preventing a disease or disorder in a subject having, or suspected of having, the disease or disorder, wherein the disease or disorder comprises an inflammatory disease or disorder or an autoimmune disease or disorder.

95. The modified IL-1 polypeptide of any of claims 1-89 or the pharmaceutical composition of any of claims 90-92 for use in treating or preventing a disease or disorder in a subject having, or suspected of having, the disease or disorder, wherein the disease or disorder comprises a proliferation disease or disorder.

96. The modified IL-1 polypeptide of any of claims 1-89 or the pharmaceutical composition of any of claims 90-92 for use in treating or preventing a disease or disorder in a subject having, or suspected of having, the disease or disorder, wherein the disease or disorder comprises an infectious disease or disorder.

97. The modified IL-1 polypeptide of any of claims 1-89 or the pharmaceutical composition of any of claims 90-92 for use in treating or preventing a disease or disorder in a subject having, or suspected of having, the disease or disorder, wherein the disease or disorder comprises an immune deficiency disorder.

98. A method for treating or preventing a disease or a disorder in a subject having, or suspected of having, the disease or disorder, comprising administering to said subject an effective amount of a modified IL-2 polypeptide of any of claims 1-89, or a pharmaceutical composition of any of claims 90-92.

99. A method for treating or preventing a disease or a disorder in a subject having, or suspected of having, the disease or disorder, comprising administering to said subject an effective amount of a modified IL-2 polypeptide of any of claims 1-89, or a pharmaceutical composition of any of claims 90-92, wherein the disease or disorder comprises an inflammatory disease or disorder or an autoimmune disease or disorder.

100. A method for treating or preventing a disease or a disorder in a subject having, or suspected of having, the disease or disorder, comprising administering to said subject an effective amount of a modified IL-2 polypeptide of any of claims 1-89, or a pharmaceutical composition of any of claims 90-92, wherein the disease or disorder comprises a proliferation disease or disorder.

101. A method for treating or preventing a disease or a disorder in a subject having, or suspected of having, the disease or disorder, comprising administering to said subject an effective amount of a modified IL-2 polypeptide of any of claims 1-89, or a pharmaceutical composition of any of claims 90-92, wherein the disease or disorder comprises an infectious disease or disorder.

102. A method for treating or preventing a disease or a disorder in a subject having, or suspected of having, the disease or disorder, comprising administering to said subject an effective amount of a modified IL-2 polypeptide of any of claims 1-89, or a pharmaceutical composition of any of claims 90-92, wherein the disease or disorder comprises an immune deficiency disease or disorder.

103. The use according to any of claims 93-97 or the method of any of claims 98-102, wherein the subject is a human.

104. The use according to any of claims 93-97 or the method of any of claims 97-102, wherein the subject is a non-human mammal.

105. The use according to claim 95 or the method of claim 100, wherein the proliferation disorder comprises a tumor.

106. The use according to claim 95 or the method of claim 100, wherein the proliferation disorder comprises a cancer.

107. The use according to claim 95 or the method of claim 100, wherein the proliferation disorder comprises a solid tumor or a cancer.

108. The use according to claim 95 or the method of claim 100, wherein the proliferation disorder comprises a solid tumor or a cancer, wherein the solid tumor or the cancer is selected from the group consisting of: Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor, and Liposarcoma.

109. The use according to claim 95 or the method of claim 100, wherein the proliferation disorder comprises a tumor or a cancer, wherein the tumor or cancer is a hematological malignancy.

110. The use according to claim 95 or the method of claim 100, wherein the proliferation disorder comprises a tumor or a cancer, wherein the tumor or cancer is a hematological malignancy selected from the group consisting of: myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenström's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schöner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenström's macroglobulinaemia, Alpha heavy chain disease, Gamma heavy chain disease, Franklin's disease, Immunoproliferative small intestinal disease, Mediterranean disease, Malignant immunoproliferative disease, unspecified, and Immunoproliferative disease NOS.

111. The use according to claim 94 or the method of claim 99, wherein the inflammatory disease or disorder or the autoimmune disease or disorder is selected from the group consisting of: inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, and transplantation rejection.

112. The use according to claim 96 or the method of claim 101, wherein the infectious disease is selected from the group consisting of: Acinetobacter infections, Actinomycosis, African sleeping sickness (African trypanosomiasis), AIDS (acquired immunodeficiency syndrome), Amoebiasis, Anaplasmosis, Angiostrongyliasis Anisakiasis, Anthrax, Arcanobacterium haemolyticum infection, Argentine hemorrhagic fever, Ascariasis, Aspergillosis Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus (children and babies), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, SARS (severe acute respiratory syndrome), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, and Zygomycosis.

113. The use according to claim 97 or the method of claim 102, wherein the immune deficiency disease or disorder is selected from the group consisting of: Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs), and Lymphopenia.

114. Use of an effective amount of a modified IL-2 polypeptide of any of claims 1-89, or an RNA polynucleotide, a DNA polynucleotide, a non-viral vector, or a viral vector encoding a modified IL-2 polypeptide of any of claims 1-89, for the manufacture of a medicament for treating or preventing a disease or a disorder in a subject.

115. The use according to claim 114, wherein the disease or disorder is selected from the group consisting of: an inflammatory disease or disorder; an autoimmune disease or disorder; a proliferative disease or disorder; an infectious disease or disorder; and an immune deficiency disease or disorder.

116. A method of expanding a Treg cell population, which comprises contacting a cell population with an effective amount of a modified IL-2 polypeptide of any of claims 1-89, or an RNA polynucleotide, a DNA polynucleotide, a non-viral vector, or a viral vector encoding a modified IL-2 polypeptide of any of claims 1-89, for a time sufficient to induce formation of a complex with an IL-2Rαβγ, thereby stimulating the expansion of the Treg cell population.

117. A method of expanding a Treg cell population, which comprises contacting a cell population with an effective amount of a modified IL-2 polypeptide of any of claims 1-89, or an RNA polynucleotide, a DNA polynucleotide, a non-viral vector, or a viral vector encoding a modified IL-2 polypeptide of any of claims 1-89, for a time sufficient to induce formation of a complex with an IL-2Rαβγ, thereby stimulating the expansion of the Treg cell population with reduced cell death by 10% to 100%.

118. The method of claim 116 or 117, wherein the effective amount causes expansion of CD25⁺ T regulatory (Treg) cells by at least 1-fold, 10-fold, 100-fold, 1000 fold, 10⁴-fold, 10⁵-fold, 10⁶-fold, 10⁷-fold, 10⁸-fold, or 10⁹-fold greater that the expansion of CD25⁺ Treg cells caused with an IL-2 polypeptide comprising an amino acid sequence set forth in SEQ ID NO:1 or SEQ ID NO:2 without the substitution.

119. The method of claim 116 or 117, wherein the effective amount causes an increased the percentage of Treg cells in the T cell population after incubation with the effective amount, compared with an IL-2 polypeptide comprising an amino acid sequence set forth in SEQ ID NO:1 or SEQ ID NO:2 without the substitution, and percentage of the Treg cells is about or at least 0.01%, 0.1%, 1%, 5%, 10%, 20%, 30%, 40%, 50%, 60%, 70%, 80%, 90% or more.

120. The method of any of claims 116-119, wherein the method is conducted in vivo.

121. The method of any of claims 116-119, wherein the method is conducted in vitro.

122. The method of any of claims 116-119, wherein the method is conducted ex vivo.

123. Use of an effective amount of a modified IL-2 polypeptide of any of claims 1-89, or an RNA polynucleotide, a DNA polynucleotide, a non-viral vector, or a viral vector encoding a modified IL-2 polypeptide of any of claims 1-89, for the manufacture of a medicament for expanding a Treg cell in a cell population.

124. The use of claim 123, wherein the Treg cells are expanded in a subject.


SEQUENCE LISTING (3 sequences)

SEQ ID NO: 1 - protein, 133 aa, Artificial Sequence
  source: /note=“Description of Artificial Sequence Synthetic polypeptide”
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 133 aa, Artificial Sequence
  source: /note=“Description of Artificial Sequence Synthetic polypeptide”
MPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - protein, 133 aa, Homo sapiens [sequence omitted]